SEQUENCE LISTING (478 sequences)

SEQ ID NO: 1 - protein, 297 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 2 - protein, 320 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDXXXXXXXXXXXXXXXXXXXXXXXXXVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 3 - protein, 319 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIXXXXXXXXXXXXXXXXXXXXXXXXXVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 4 - protein, 322 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVXXXXXXXXXXXXXXXXXXXXXXXXXRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 5 - protein, 322 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPXXXXXXXXXXXXXXXXXXXXXXXXXLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 6 - protein, 163 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLII

SEQ ID NO: 7 - protein, 164 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIID

SEQ ID NO: 8 - protein, 165 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQ

SEQ ID NO: 9 - protein, 166 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQN

SEQ ID NO: 10 - protein, 134 aa, Synthetic construct
DQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 11 - protein, 133 aa, Synthetic construct
QNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 12 - protein, 132 aa, Synthetic construct
NVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 13 - protein, 131 aa, Synthetic construct
VFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 14 - protein, 176 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMG

SEQ ID NO: 15 - protein, 177 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGV

SEQ ID NO: 16 - protein, 178 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVV

SEQ ID NO: 17 - protein, 179 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVR

SEQ ID NO: 18 - protein, 180 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRP

SEQ ID NO: 19 - protein, 181 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPL

SEQ ID NO: 20 - protein, 182 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLT

SEQ ID NO: 21 - protein, 121 aa, Synthetic construct
VVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 22 - protein, 120 aa, Synthetic construct
VRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 23 - protein, 119 aa, Synthetic construct
RPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 24 - protein, 118 aa, Synthetic construct
PLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 25 - protein, 117 aa, Synthetic construct
LTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 26 - protein, 116 aa, Synthetic construct
TEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 27 - protein, 115 aa, Synthetic construct
EVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 28 - protein, 183 aa, Synthetic construct
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 29 - protein, 165 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 30 - protein, 11 aa, Synthetic construct
VTGYRLFEEIL

SEQ ID NO: 31 - protein, 11 aa, Synthetic construct
VSGWRLFKKIS

SEQ ID NO: 32 - protein, 11 aa, Synthetic construct
NVFIEGTLPMG

SEQ ID NO: 33 - protein, 478 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 34 - protein, 480 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 35 - protein, 468 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILARPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 36 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 37 - protein, 479 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSADQMAQIEEVFKVVYPVDDHHFKVILPYGTLVIDGVTPNKLNYFGHPYEGIAVFDGEKITVTGTLWNGNKIIDERLITPDGSMLFRVTINGVSGWRLFKKISGGTGGSGGTGGSMVFTLDDFVGDWEQTAAYNLDQVLEQGGVSSLLQNLAVSVTPIQRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 38 - protein, 479 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSADQMAQIEEVFKVVYPVDDHHFKVILPYGTLVIDGVTPNKLNYFGHPYEGIAVFDGEKITVTGTLWNGNKIIDERLITPDGSMLFRVTINGVSGWRLCKKISGGTGGSGGTGGSMVFTLDDFVGDWEQTAAYNLDQVLEQGGVSSLLQNLAVSVTPIQRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 39 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLERPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 40 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 41 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 42 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 43 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 44 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 45 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 46 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 47 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 48 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 49 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 50 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 51 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 52 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 53 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 54 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIARPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 55 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 56 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 57 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVNPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLIRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 58 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 59 - protein, 483 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 60 - protein, 486 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 61 - protein, 489 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 62 - protein, 492 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGSSSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 63 - protein, 495 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGSSSGGSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 64 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 65 - protein, 474 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGGMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 66 - protein, 477 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGGGSGMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 67 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGGGSGGSSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 68 - protein, 483 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGSGGGGSGGSSSGGMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 69 - protein, 483 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLGGSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 70 - protein, 486 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLGGSGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 71 - protein, 489 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLGGSGGGGSGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 72 - protein, 492 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLGGSGGGGSGGSSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 73 - protein, 495 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLGGSGGGGSGGSSSGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 74 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDHNVFIEGTLRMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 75 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDIGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 76 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDIGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPADREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 77 - protein, 480 aa, Synthetic construct
GSEIGTGFPFDPHYVEVLGERLHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 78 - protein, 480 aa, Synthetic construct
GSEIGTGFPFDPHYVEVLGERMHYVDVGPRDGTPVLFLHGNPTSSYVWRNIIPHVAPTHRCIAPDLIGFGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 79 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFANELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 80 - protein, 480 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEFPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 81 - protein, 192 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREP

SEQ ID NO: 82 - protein, 193 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPF

SEQ ID NO: 83 - protein, 194 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFL

SEQ ID NO: 84 - protein, 195 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLN

SEQ ID NO: 85 - protein, 196 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNP

SEQ ID NO: 86 - protein, 105 aa, Synthetic construct
FLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 87 - protein, 104 aa, Synthetic construct
LNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 88 - protein, 103 aa, Synthetic construct
NPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 89 - protein, 102 aa, Synthetic construct
PVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 90 - protein, 101 aa, Synthetic construct
VDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 91 - protein, 468 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSADQMAQIEEVFKVVYPVDDHHFKVILPYGTLVIDGVTPNMLNYFGRPYEGIAVFDGKKITVTGTLWNGNKIIDERLITPDGSMLFRVTINSGGTGGSGGTGGSMVFTLEDFVGDWEQTAAYNLDQVLEQGGVSSLLQNLAVSVTPIQRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 92 - protein, 468 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 93 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 94 - protein, 474 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLGGSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 95 - protein, 483 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGSSSGGSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 96 - protein, 468 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGENALKIDIHVIIPYEGLSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 97 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGENALKIDIHVIIPYEGLSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 98 - protein, 474 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGENALKIDIHVIIPYEGLSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGGSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 99 - protein, 483 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGSSSGGGENALKIDIHVIIPYEGLSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 100 - protein, 471 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLGGSSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 101 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGRPYEGIAVFDGEKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 102 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGHPYEGIAVFDGEKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 103 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGRPYEGIAVFDGEKITTTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 104 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGHPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 105 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGHPYEGIAVFDGKKITTTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 106 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGHPYEGIAVFDGEKITTTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 107 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGRPYEGIAVFDGKKITTTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 108 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDRHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 109 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDRHFKVILPYGTLVIDGVTPNKLNYFGRPYEGIAVFDGKKITVTGTLWNGNKIIDERRIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 110 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQLEEVFKVVYPVDDHHFKVILPYGTLVIDGVTPNKLNYFGRPYEGIAVFDGKKITVTGTLWNGNKIIDERRIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 111 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQLEEVFKVVYPVDDRHFKVILPYGTLVIDGVTPNKLNYFGRPYEGIAVFDGKKITVTGTLWNGNKIIDERRIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 112 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQLEEVFKVVYPVDDRHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 113 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQLEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 114 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGRPYEGIAVFDGKKITVTGTLWNGNKIIDERRIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 115 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGPRSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 116 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRPSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 117 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPGSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 118 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVVPRSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 119 - protein, 471 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVVRPSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 120 - protein, 481 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVEPTTEDLYFQSDNSADQMAQIEEVFKVVYPVDDHHFKVILPYGTLVIDGVTPNKLNYFGRPYEGIAVFDGKKITVTGTLWNGNKIIDERLIDPDGSMLFRVTINSGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 121 - protein, 656 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSGMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGS

SEQ ID NO: 122 - protein, 514 aa, Synthetic construct
MLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSSGGGSSGGEPTTENLYFQSDNGSSGGGSSGGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLSGENGLKIDIHVIIPYEGL

SEQ ID NO: 123 - protein, 514 aa, Synthetic construct
MLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGSSGGGSSGGEPTTENLYFQSDNGSSGGGSSGGMAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGTGGSGGTGGSMVFTLEDFVGDWRQTAGYNLDQVLEQGGVSSLFQNLGVSVTPIQRIVLS

SEQ ID NO: 124 - protein, 456 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVSADQMAQIEEVFKVVYPVDDHHFKVILPYGTLVIDGVTPNKLNYFGHPYEGIAVFDGEKITTTGTLWNGNKIIDERLIDPDGGTGGSGGTGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 125 - protein, 460 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGHPYEGIAVFDGEKITTTGTLWNGNKIIDERLIDPDGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 126 - protein, 463 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSSADQMAQIEEVFKVVYPVDDHHFKVILPYGTLVIDGVTPNKLNYFGHPYEGIAVFDGEKITTTGTLWNGNKIIDERLIDPDGGTGGSGGTGGSMVFTLDDFVGDWEQTAAYNLDQVLEQGGVSSLLQNLAVSVTPIMRIVRSGENALKIDIHVIIPYEGLGGSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 127 - protein, 472 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGSSSGGSADQMAQIEEVFKVVYPVDDHHFKVILPYGTLVIDGVTPNKLNYFGHPYEGIAVFDGEKITTTGTLWNGNKIIDERLIDPDGGTGGSGGTGGSMVFTLDDFVGDWEQTAAYNLDQVLEQGGVSSLLQNLAVSVTPIMRIVRSGENALKIDIHVIIPYEGLRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 128 - protein, 412 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVDDTHRKCPEDRHPCHHPVRSERRPNGPDRRGVGGVPCGSSLGDPALWHTGNRRGYAEQAELFRTPVRHRRVRRREDHYHRDPVERQQNYRRAPDRSRRRNRWQRWNREHGLHTRRFRWGLGTDSRLQPGPSPTGRCVQFAAESRRVRNSDHEDCPEPLVEEYMDWLHQSPVPKLLFWGTPGVLIPPAEAARLAKSLPNCKAVDIGPGLNLLQEDNPDLIGSEIARWLSTLEISG

SEQ ID NO: 129 - protein, 460 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGENALKIDIHVIIPYEGLSADQMAQIEEVFKVVYPVDDHHFKVILPYGTLVIDGVTPNKLNYFGHPYEGIAVFDGEKITTTGTLWNGNKIIDERLIDPDGGTGGSGGTGGSMVFTLDDFVGDWEQTAAYNLDQVLEQGGVSSLLQNLAVSVTPIMRIVRSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 130 - protein, 463 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGENALKIDIHVIIPYEGLSADQMAQIEEVFKVVYPVDDHHFKVILPYGTLVIDGVTPNKLNYFGHPYEGIAVFDGEKITTTGTLWNGNKIIDERLIDPDGGTGGSGGTGGSMVFTLDDFVGDWEQTAAYNLDQVLEQGGVSSLLQNLAVSVTPIMRIVRSGGSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 131 - protein, 472 aa, Synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGSSSGGGENALKIDIHVIIPYEGLSADQMAQIEEVFKVVYPVDDHHFKVILPYGTLVIDGVTPNKLNYFGHPYEGIAVFDGEKITTTGTLWNGNKIIDERLIDPDGGTGGSGGTGGSMVFTLDDFVGDWEQTAAYNLDQVLEQGGVSSLLQNLAVSVTPIMRIVRSRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 132 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLVFIPPLWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 133 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSDRVMTYLIDKWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 134 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSFRKPYYFWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 135 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSWDLVLFLWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 136 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSRVGVYQAWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 137 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSTRYCLFSWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 138 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSYMTIYDLLHGWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 139 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLKLDIGSDMGWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 140 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSFSMFPAGFLWWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 141 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSRSTHHNRWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 142 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSHDILYYSKKTWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 143 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSQAGINDLSSVWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 144 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSNVDCKMTSILWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 145 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSDYPNVPYWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 146 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSPTYIAHTISTWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 147 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLTRPMHNLNAWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 148 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKTLNIMNWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 149 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSFKMTVFRWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 150 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSHGNPKNICDHWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 151 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSAIISRTQWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 152 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLDDCPTMSKYWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 153 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKSLKNKVKLVWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 154 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLRVDDNHWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 155 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLNTSTVYWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 156 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSFTTLRESWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 157 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSNNTNTMDWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 158 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSHWANHACTKTWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 159 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSTALEPLTWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 160 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSSNTDLMCERKWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 161 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLENPLSGWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 162 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSSHISKGNSERWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 163 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSDANGHSSSSVWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 164 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSTLSCHLGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 165 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRPKPTSAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 166 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGMGRESVHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 167 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGASDCNSTNSASGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 168 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGTHLIEYNKQKAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 169 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGITWAGSSPASPIRYDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 170 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSGSLSNRQWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 171 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSHPYLTGQWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 172 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSRARLEPSWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 173 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSQVFYTPVWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 174 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLMLSYPRWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 175 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSPHMLERYWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 176 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSWKISDTTNNGNWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 177 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSPLTITIKLGNPWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 178 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLSKNVHPTVPYWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 179 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSDMPLVEIRYKVWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 180 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLHINPPKASANWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 181 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSESLRTHTGNNAPHSVWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 182 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSAWPKPVTYQISYSQHWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 183 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSQRYAPTLITYLYNTSWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 184 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSLANVDSTHCHNTPNYWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 185 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGPTCGYSLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 186 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLYRGMRLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 187 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRVTYKMRMPVTGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 188 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGYISVNFIQDSDRCVVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 189 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVCLVLTARVFWEQLGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 190 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGHYVTSMDLATAVVVLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 191 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSATKCGYVWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 192 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSVVAREWEWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 193 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSFDEVGMTSNGVWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 194 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSRMCGVSFRVLLWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 195 - protein, 318 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSCHWKVALTFSDVPDYWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 196 - protein, 327 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSVSDIAQTWGVFIEGTLPMGVVGSVPQYWPAFITTLSYAWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 197 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSYLLDPPKWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 198 - protein, 308 aa, Synthetic construct; identical to SEQ ID NO: 197

SEQ ID NO: 199 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVPSWFRYGTLWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 200 - protein, 313 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVHHHHHGSGQSYTWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 201 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVHHHHHGSGMPWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 202 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVHHHHHGSGMGRCGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 203 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQLPPFNPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 204 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPALLDHRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 205 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGVCIYPMAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 206 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGFCTLLNLGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 207 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKYGPQCGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 208 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLKHLRSIGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 209 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWPPATAAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 210 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGNPTEGPEGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 211 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPMFTNHYGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 212 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHLQQSRIGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 213 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSHSLDKQGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 214 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHDARWHTGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 215 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGTFPQHPSGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 216 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGAKQKKFNGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 217 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGTKFDYTFGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 218 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGMTVSTQFGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 219 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQAHSDTGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 220 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSTTLMHPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 221 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGALTKDSNGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 222 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQTSPLTGGTPVGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 223 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPPGHDHVRLTHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 224 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRNNWILCGTPTGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 225 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGIMTNSRDSRRNGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 226 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLRPILTYKPTHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 227 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPKFHYRSTTSGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 228 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSQVNPSHWSLHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 229 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRCIPYHMYHRTGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 230 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGKGPKAAGADNFGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 231 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPISAIRWQKLYGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 232 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLTRYNKFATKRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 233 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWLATFAQDKMDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 234 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLPGDDHVRMTSGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 235 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGALSMDTFQYFQGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 236 - protein, 309 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSQFFSIGLWVGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 237 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGYSFALRRLGLPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 238 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWTIYNLIEPNEGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 239 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGVPTHPGHTWLRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 240 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGMNNHWLVVTPNQSGYGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 241 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLSLFWAKVMLGIHCRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 242 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGVHGSMQHRNGMKYCNGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 243 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSHYSSPCCIPPVETRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 244 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQTSPLNNKNLACLRDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 245 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSYLKSRHVKFIGHLMGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 246 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGFDSGVTLIDKANGLMGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 247 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGFLMSNFRYDCLTNTSGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 248 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGENPYSPFRNRNTAPHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 249 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGFERANCTDYYHSRPWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 250 - protein, 314 aa, Synthetic construct; identical to SEQ ID NO: 248

SEQ ID NO: 251 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWAVAAYLNSWYMVKKGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 252 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPFRERPGDRSIYGETGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 253 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPLSVQPDKCGKSMLRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 254 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGYCQDFLCRLINTRLKGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 255 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSSIYDFEVYKAESSRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 256 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGMKSLYISLMRLLGIRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 257 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLAWTERYWPVPRVKDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 258 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGMLYFKWRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 259 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLRPRCGVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 260 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGPMLRFAAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 261 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGKSLRDDAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 262 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGNAKEETGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 263 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGNMGIDVWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 264 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRQIVMVWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 265 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSRWYCTCGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 266 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGWKFVFALGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 267 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRSTISYGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 268 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGWNVRLRWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 269 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGEIGANGEGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 270 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGCEVNRCRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 271 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGDWNIVSCGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 272 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLGTMLLGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 273 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGNMSFQCGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 274 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRSGVTFGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 275 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGAHIGVDAVLGLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 276 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGKPSVHGNVHLAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 277 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVPYDGRTLCVVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 278 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGYIAGVMMVGVGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 279 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGFGAGSRWLLRDGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 280 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLRRFQKWTKFDGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 281 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGDIGYVLGTLMRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 282 - protein, 311 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVLMLGDGDVCGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 283 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLSGCWYSGEFEGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 284 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVACGWMALRMIGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 285 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRLFEALGSLAVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 286 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGQYFQFDGVYKTGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 287 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGHRGFVVTVRGLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 288 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVQSGSWLVIGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 289 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGESSLCLLRVVWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 290 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGPVLMSCDFRPHGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 291 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSHTTSFAHGRRSLSRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 292 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGFIISTLGLRSFYCPLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 293 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSFYSIWADFRRASVLGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 294 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRSILNFAFMFPRTFYGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 295 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLTRGINAAILSPCLQGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 296 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGVTSLRPTLRSGVTVSGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 297 - protein, 316 aa, Synthetic construct; identical to SEQ ID NO: 291

SEQ ID NO: 298 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGTMVSIWLLCLIMERRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 299 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGDRDSWFSLKYVWEYVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 300 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGCWCGMESHSSSAYDKGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 301 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGTRGGPNECWAEVHIIGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 302 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGFPTSICPLFGWFGSQGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 303 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRADGIANMGTSVGWAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 304 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSARRVNSRSLAMSQKGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 305 - protein, 316 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGSVLVYPDNVIIWHPGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 306 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKTVKTAGVFIEGTLPMGVVGRRAGRIRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 307 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGTRPSRFSGVFIEGTLPMGVVGVGWMRSSGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 308 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWITFERNGVFIEGTLPMGVVGRESDGWSGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 309 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLKTRLTSGVFIEGTLPMGVVGLDLHLPMGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 310 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGTTTGRNPGVFIEGTLPMGVVGWKRCVGQGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 311 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGGFEWDGNGVFIEGTLPMGVVGRTVWLLSGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 312 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSVGNTDAGVFIEGTLPMGVVGCTHAIDGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 313 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGASETKDSGVFIEGTLPMGVVGSFQEFMVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 314 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPPCFNMCGVFIEGTLPMGVVGLVQLRMCGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 315 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGNHMLQTTGVFIEGTLPMGVVGGKMYMQGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 316 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHEPEAQRGVFIEGTLPMGVVGCGMDELVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 317 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGTSMLNFTGVFIEGTLPMGVVGRCSFEAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 318 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRPPDFTGGVFIEGTLPMGVVGGGVFQGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 319 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPSSRQQYGVFIEGTLPMGVVGSIHADGVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 320 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGNGIVVTHGVFIEGTLPMGVVGELAKLRRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 321 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWAGRASKGVFIEGTLPMGVVGLSSRNLKGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 322 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRKVTMKKGVFIEGTLPMGVVGGKGVFCSGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 323 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSYFSLKVGVFIEGTLPMGVVGLAEHKRRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 324 - protein, 299 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 325 - protein, 299 aa, Synthetic construct; identical to SEQ ID NO: 324

SEQ ID NO: 326 - protein, 299 aa, Synthetic construct; identical to SEQ ID NO: 324

SEQ ID NO: 327 - protein, 299 aa, Synthetic construct; identical to SEQ ID NO: 324

SEQ ID NO: 328 - protein, 299 aa, Synthetic construct; identical to SEQ ID NO: 324

SEQ ID NO: 329 - protein, 299 aa, Synthetic construct; identical to SEQ ID NO: 324

SEQ ID NO: 330 - protein, 299 aa, Synthetic construct; identical to SEQ ID NO: 324

SEQ ID NO: 331 - protein, 299 aa, Synthetic construct; identical to SEQ ID NO: 324

SEQ ID NO: 332 - protein, 299 aa, Synthetic construct; identical to SEQ ID NO: 324

SEQ ID NO: 333 - protein, 299 aa, Synthetic construct; identical to SEQ ID NO: 324

SEQ ID NO: 334 - protein, 299 aa, Synthetic construct; identical to SEQ ID NO: 324

SEQ ID NO: 335 - protein, 299 aa, Synthetic construct; identical to SEQ ID NO: 324

SEQ ID NO: 336 - protein, 299 aa, Synthetic construct; identical to SEQ ID NO: 324

SEQ ID NO: 337 - protein, 299 aa, Synthetic construct; identical to SEQ ID NO: 324

SEQ ID NO: 338 - protein, 299 aa, Synthetic construct; identical to SEQ ID NO: 324

SEQ ID NO: 339 - protein, 299 aa, Synthetic construct; identical to SEQ ID NO: 324

SEQ ID NO: 340 - protein, 299 aa, Synthetic construct; identical to SEQ ID NO: 324

SEQ ID NO: 341 - protein, 299 aa, Synthetic construct; identical to SEQ ID NO: 324

SEQ ID NO: 342 - protein, 299 aa, Synthetic construct; identical to SEQ ID NO: 324

SEQ ID NO: 343 - protein, 299 aa, Synthetic construct; identical to SEQ ID NO: 324

SEQ ID NO: 344 - protein, 299 aa, Synthetic construct; identical to SEQ ID NO: 324

SEQ ID NO: 345 - protein, 299 aa, Synthetic construct; identical to SEQ ID NO: 324

SEQ ID NO: 346 - protein, 299 aa, Synthetic construct; identical to SEQ ID NO: 324

SEQ ID NO: 347 - protein, 299 aa, Synthetic construct; identical to SEQ ID NO: 324

SEQ ID NO: 348 - protein, 299 aa, Synthetic construct; identical to SEQ ID NO: 324

SEQ ID NO: 349 - protein, 299 aa, Synthetic construct; identical to SEQ ID NO: 324

SEQ ID NO: 350 - protein, 299 aa, Synthetic construct; identical to SEQ ID NO: 324

SEQ ID NO: 351 - protein, 299 aa, Synthetic construct; identical to SEQ ID NO: 324

SEQ ID NO: 352 - protein, 299 aa, Synthetic construct; identical to SEQ ID NO: 324

SEQ ID NO: 353 - protein, 299 aa, Synthetic construct; identical to SEQ ID NO: 324

SEQ ID NO: 354 - protein, 299 aa, Synthetic construct; identical to SEQ ID NO: 324

SEQ ID NO: 355 - protein, 299 aa, Synthetic construct; identical to SEQ ID NO: 324

SEQ ID NO: 356 - protein, 299 aa, Synthetic construct; identical to SEQ ID NO: 324

SEQ ID NO: 357 - protein, 299 aa, Synthetic construct; identical to SEQ ID NO: 324

SEQ ID NO: 358 - protein, 299 aa, Synthetic construct; identical to SEQ ID NO: 324

SEQ ID NO: 359 - protein, 299 aa, Synthetic construct; identical to SEQ ID NO: 324

SEQ ID NO: 360 - protein, 299 aa, Synthetic construct; identical to SEQ ID NO: 324

SEQ ID NO: 361 - protein, 299 aa, Synthetic construct; identical to SEQ ID NO: 324

SEQ ID NO: 362 - protein, 299 aa, Synthetic construct; identical to SEQ ID NO: 324

SEQ ID NO: 363 - protein, 299 aa, Synthetic construct; identical to SEQ ID NO: 324

SEQ ID NO: 364 - protein, 299 aa, Synthetic construct; identical to SEQ ID NO: 324

SEQ ID NO: 365 - protein, 299 aa, Synthetic construct; identical to SEQ ID NO: 324

SEQ ID NO: 366 - protein, 299 aa, Synthetic construct; identical to SEQ ID NO: 324

SEQ ID NO: 367 - protein, 299 aa, Synthetic construct; identical to SEQ ID NO: 324

SEQ ID NO: 368 - protein, 299 aa, Synthetic construct; identical to SEQ ID NO: 324

SEQ ID NO: 369 - protein, 299 aa, Synthetic construct; identical to SEQ ID NO: 324

SEQ ID NO: 370 - protein, 299 aa, Synthetic construct; identical to SEQ ID NO: 324

SEQ ID NO: 371 - protein, 299 aa, Synthetic construct; identical to SEQ ID NO: 324

SEQ ID NO: 372 - protein, 299 aa, Synthetic construct; identical to SEQ ID NO: 324

SEQ ID NO: 373 - protein, 299 aa, Synthetic construct; identical to SEQ ID NO: 324

SEQ ID NO: 374 - protein, 299 aa, Synthetic construct; identical to SEQ ID NO: 324

SEQ ID NO: 375 - protein, 299 aa, Synthetic construct; identical to SEQ ID NO: 324

SEQ ID NO: 376 - protein, 299 aa, Synthetic construct; identical to SEQ ID NO: 324

SEQ ID NO: 377 - protein, 299 aa, Synthetic construct; identical to SEQ ID NO: 324

SEQ ID NO: 378 - protein, 299 aa, Synthetic construct; identical to SEQ ID NO: 324

SEQ ID NO: 379 - protein, 299 aa, Synthetic construct; identical to SEQ ID NO: 324

SEQ ID NO: 380 - protein, 299 aa, Synthetic construct; identical to SEQ ID NO: 324

SEQ ID NO: 381 - protein, 299 aa, Synthetic construct; identical to SEQ ID NO: 324

SEQ ID NO: 382 - protein, 299 aa, Synthetic construct; identical to SEQ ID NO: 324

SEQ ID NO: 383 - protein, 299 aa, Synthetic construct; identical to SEQ ID NO: 324

SEQ ID NO: 384 - protein, 299 aa, Synthetic construct; identical to SEQ ID NO: 324

SEQ ID NO: 385 - protein, 299 aa, Synthetic construct; identical to SEQ ID NO: 324

SEQ ID NO: 386 - protein, 299 aa, Synthetic construct; identical to SEQ ID NO: 324

SEQ ID NO: 387 - protein, 299 aa, Synthetic construct; identical to SEQ ID NO: 324

SEQ ID NO: 388 - protein, 299 aa, Synthetic construct; identical to SEQ ID NO: 324

SEQ ID NO: 389 - protein, 299 aa, Synthetic construct; identical to SEQ ID NO: 324

SEQ ID NO: 390 - protein, 299 aa, Synthetic construct; identical to SEQ ID NO: 324

SEQ ID NO: 391 - protein, 299 aa, Synthetic construct; identical to SEQ ID NO: 324

SEQ ID NO: 392 - protein, 299 aa, Synthetic construct; identical to SEQ ID NO: 324

SEQ ID NO: 393 - protein, 299 aa, Synthetic construct; identical to SEQ ID NO: 324

SEQ ID NO: 394 - protein, 299 aa, Synthetic construct; identical to SEQ ID NO: 324

SEQ ID NO: 395 - protein, 299 aa, Synthetic construct; identical to SEQ ID NO: 324

SEQ ID NO: 396 - protein, 299 aa, Synthetic construct; identical to SEQ ID NO: 324

SEQ ID NO: 397 - protein, 299 aa, Synthetic construct; identical to SEQ ID NO: 324

SEQ ID NO: 398 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGANFTSNWLDDRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 399 - protein, 310 aa, Synthetic construct; identical to SEQ ID NO: 398

SEQ ID NO: 400 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGCDKTDTLFEGAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 401 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGCHPNTLHMQGHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 402 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGDLLLGMIRPSLGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 403 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGKPSTSNKQYTPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 404 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPLRVQLNSLVIGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 405 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQPRTVPSFFDPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 406 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSNMDTVLKRLKGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 407 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGYQKQSWPLPMWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 408 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGMPTPTVPYTSPRLTPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 409 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGEHMTLLAGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 410 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGESGGRSCGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 411 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGIVELLWMGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 412 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLLYWLLQGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 413 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGMWMQRPSGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 414 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGPTETEFWGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 415 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRGRGKSKGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 416 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLPACGFYCLMMGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 417 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGLVYLACLGMASGGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 418 - protein, 315 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGTMSTGLEFRGAVVVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 419 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGAPVASFQGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 420 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGMMSACMVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 421 - protein, 308 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGMPRDMEMGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 422 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGADNDIDAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 423 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGATLFSQEGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 424 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGCKSYTTEGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 425 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGDHPRDCYGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 426 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGDLDAILTGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 427 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHLVPPLWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 428 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHPRNLTDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 429 - protein, 306 aa, Synthetic construct; identical to SEQ ID NO: 428

SEQ ID NO: 430 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHRQPRHRGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 431 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGIAVHYVDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 432 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGKSYLNSGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 433 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLTSISNAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 434 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGNEPLTNTGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 435 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGNTFLQTAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 436 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGPSKSETGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 437 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQLLMNVEGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 438 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRPITPAAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 439 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRPWVKVKGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 440 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGRVHLPVPGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGS

SEQ ID NO: 441 - protein, 299 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQGGNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 442 - protein, 302 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQGSGSGNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 443 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQGGSGGGGSGGNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 444 - protein, 299 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPGGLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 445 - protein, 302 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPGSGSGLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 446 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPGGSGGGGSGGLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 447 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGGSGGGGSGGQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 448 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIGGSGGGGSGGDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 449 - protein, 307 aa, Synthetic construct; identical to SEQ ID NO: 448

SEQ ID NO: 450 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVGGSGGGGSGGFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 451 - protein, 305 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGGSGGGGSGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 452 - protein, 304 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIGGSGGGGSGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 453 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 454 - protein, 307 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGGGSGGGGSGGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 455 - protein, 304 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRGGSGGGGSGGEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 456 - protein, 303 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 457 - protein, 304 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGGSGGGGSGGTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 458 - protein, 304 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWPPATAAGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 459 - protein, 304 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGHDARWHTGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 460 - protein, 309 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGWTIYNLIEPNEGGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 461 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGENPYSPFRNRNTAPHGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 462 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGFERANCTDYYHSRPWGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 463 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGYCQDFLCRLINTRLKGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 464 - protein, 312 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGLAWTERYWPVPRVKDGVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 465 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGNMGIDVWGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 466 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGCEVNRCRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 467 - protein, 306 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGRSGVTFGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 468 - protein, 310 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGKPSVHGNVHLAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 469 - protein, 314 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVGDRDSWFSLKYVWEYVGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 470 - protein, 313 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKYGPQCGVFIEGTLPMGVVGRSGVTFGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 471 - protein, 317 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKYGPQCGVFIEGTLPMGVVGKPSVHGNVHLAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 472 - protein, 321 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGSKYGPQCGVFIEGTLPMGVVGSHTTSFAHGRRSLSRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 473 - protein, 313 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQAHSDTGGVFIEGTLPMGVVGRSGVTFGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 474 - protein, 317 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQAHSDTGGVFIEGTLPMGVVGKPSVHGNVHLAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 475 - protein, 321 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGQAHSDTGGVFIEGTLPMGVVGSHTTSFAHGRRSLSRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 476 - protein, 321 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGENPYSPFRNRNTAPHGVFIEGTLPMGVVGRSGVTFGGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 477 - protein, 325 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGENPYSPFRNRNTAPHGVFIEGTLPMGVVGKPSVHGNVHLAGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 478 - protein, 329 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDGENPYSPFRNRNTAPHGVFIEGTLPMGVVGSHTTSFAHGRRSLSRGRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

(19) United States
(12) Patent Application Publication
(10) Pub. No.: US 2024/0132859 A1
(43) Pub. Date: Apr. 25, 2024

(54) MODIFIED DEHALOGENASE WITH EXTENDED SURFACE LOOP REGIONS

(71) Applicant: Promega Corporation, Madison, WI (US)

(72) Inventors: Michael P. Killoran, Madison, WI (US); Lance P. Encell, Madison, WI (US); Thomas Kirkland, Madison, WI (US); Thomas Machleidt, Madison, WI (US); Rachel Friedman Ohana, Madison, WI (US); Robin Hurst, Madison, WI (US); Mark A. Klein, Madison, WI (US); Karilyn Porter, Madison, WI (US); Rahele Esmatpour, Madison, WI (US); Debayan De Bakshi, Madison, WI (US)

(21) Appl. No.: 18/312,441

(22) Filed: May 4, 2023

Related U.S. Application Data
(60) Provisional application No. 63/338,369, filed on May 4, 2022.

(51) Int. Cl. C12N 9/14

(52) U.S. Cl. CPC C12N 9/14; C12Y 308/01

(57) ABSTRACT

Provided herein modified dehalogenases have extended surface loop regions that provide a location for internal fusion insertions and modulate binding interaction and activation of environmentally-sensitive chemistries.

### CROSS-REFERENCE TO RELATED APPLICATIONS

This application claims the benefit of U.S. Provisional Patent Application No. 63/338,369, filed on May 4, 2022, which is incorporated by reference herein.

### FIELD

Provided herein are modified dehalogenases that have extended surface loop regions that provide a location for internal fusion insertions and modulate binding interaction and activation of environmentally-sensitive chemistries.

### Mega Table

The specification includes a lengthy table. Table 1 has been submitted via EFS-Web in electronic format as follows: File name: TABLE_1_Loop_HTs.txt, Date created: May 4, 2023, 2023, File size: 117,291 Bytes. The content of Table 1 is hereby incorporated by reference in its entirety.

### BACKGROUND

The utility of self-labeling protein systems, such as HALOTAG and its chloroalkane-based ligands, has continually expanded during the lifetime of this research tool. Genetic fusions to HALOTAG as a general strategy has enabled a broad range of applications including fluorescence labeling for cell biology and imaging, recombinant protein purification, biosensors and diagnostics, energy transfer technologies (BRET, FRET), and targeted protein degradation assays for therapeutics (PROTACs).

What is needed are modified HALOTAG proteins that provide substrate interactions, optimal molecular proximity, or optimal molecular geometry

### SUMMARY

Provided herein are modified dehalogenases with extended surface loop regions that provide a location for internal fusion insertions and modulate binding interaction and activation of environmentally-sensitive chemistries.

In some embodiments, provided herein are compositions comprising a polypeptide having at least 70% (e.g., 70%, 75%, 80%, 85%, 90%, 95%, 100%, or ranges therebetween) sequence identity with SEQ ID NO: 2, wherein each of X₁-X₂₅ is independently selected from any amino acid or absent, wherein at least 5 of X₁-X₂₅ are not absent, wherein the polypeptide has less than 100% sequence identity with SEQ ID NO: 1. In some embodiments, at least 10 of X₁-X₂₅ are not absent.

In some embodiments, provided herein are compositions comprising a polypeptide having at least 70% (e.g., 70%, 75%, 80%, 85%, 90%, 95%, 100%, or ranges therebetween) sequence identity with SEQ ID NO: 3, wherein each of X₁-X₂₅ is independently selected from any amino acid or absent, wherein at least 5 of X₁-X₂₅ are not absent, wherein the polypeptide has less than 100% sequence identity with SEQ ID NO: 1. In some embodiments, at least 10 of X₁-X₂₅ are not absent.

In some embodiments, provided herein are compositions comprising a polypeptide having at least 70% (e.g., 70%, 75%, 80%, 85%, 90%, 95%, 100%, or ranges therebetween) sequence identity with SEQ ID NO: 4, wherein each of X₁-X₂₅ is independently selected from any amino acid or absent, wherein at least 5 of X₁-X₂₅ are not absent, wherein the polypeptide has less than 100% sequence identity with SEQ ID NO: 1. In some embodiments, at least 10 of X₁-X₂₅ are not absent.

In some embodiments, provided herein are compositions comprising a polypeptide having at least 70% sequence identity with SEQ ID NO: 5, wherein each of X₁-X₂₅ is independently selected from any amino acid or absent, wherein at least 5 of X₁-X₂₅ are not absent, wherein the polypeptide has less than 100% sequence identity with SEQ ID NO: 1.

In some embodiments, at least 10 of X₁-X₂₅ are not absent.

In some embodiments, provided herein are compositions comprising a polypeptide having an N-terminal segment comprising at least 70% (e.g., 70%, 75%, 80%, 85%, 90%, 95%, 100%, or ranges therebetween) sequence identity with one of SEQ ID NO: 6-9, a C-terminal segment comprising at least 70% (e.g., 70%, 75%, 80%, 85%, 90%, 95%, 100%, or ranges therebetween) sequence identity with SEQ ID NO 10-13, and an internal segment linking the N-terminal and C-terminal segments, wherein the internal segment is greater than 25 amino acids in length. In some embodiments, the polypeptide comprises an N-terminal segment comprising at least 70% (e.g., 70%, 75%, 80%, 85%, 90%, 95%, 100%, or ranges therebetween) sequence identity with SEQ ID NO: 6, a C-terminal segment comprising at least 70% (e.g., 70%, 75%, 80%, 85%, 90%, 95%, 100%, or ranges therebetween) sequence identity with SEQ ID NO 10, and an internal segment linking the N-terminal and C-terminal segments, wherein the internal segment is greater than 25 amino acids in length. In some embodiments, the polypeptide comprises an N-terminal segment comprising at least 70% (e.g., 70%, 75%, 80%, 85%, 90%, 95%, 100%, or ranges therebetween) sequence identity with SEQ ID NO: 7, a C-terminal segment comprising at least 70% (e.g., 70%, 75%, 80%, 85%, 90%, 95%, 100%, or ranges therebetween) sequence identity with SEQ ID NO 11, and an internal segment linking the N-terminal and C-terminal segments, wherein the internal segment is greater than 25 amino acids in length. In some embodiments, the polypeptide comprises an N-terminal segment comprising at least 70% (e.g., 70%, 75%, 80%, 85%, 90%, 95%, 100%, or ranges therebetween) sequence identity with SEQ ID NO: 8, a C-terminal segment comprising at least 70% (e.g., 70%, 75%, 80%, 85%, 90%, 95%, 100%, or ranges therebetween) sequence identity with SEQ ID NO 12, and an internal segment linking the N-terminal and C-terminal segments, wherein the internal segment is greater than 25 amino acids in length. In some embodiments, the polypeptide comprises an N-terminal segment comprising at least 70% (e.g., 70%, 75%, 80%, 85%, 90%, 95%, 100%, or ranges therebetween) sequence identity with SEQ ID NO: 9, a C-terminal segment comprising at least 70% (e.g., 70%, 75%, 80%, 85%, 90%, 95%, 100%, or ranges therebetween) sequence identity with SEQ ID NO 13, and an internal segment linking the N-terminal and C-terminal segments, wherein the internal segment is greater than 25 amino acids in length.

In some embodiments, provided herein are compositions comprising a polypeptide having an N-terminal segment comprising at least 70% (e.g., 70%, 75%, 80%, 85%, 90%, 95%, 100%, or ranges therebetween) sequence identity with one of SEQ ID NO: 14-20, a C-terminal segment comprising at least 70% (e.g., 70%, 75%, 80%, 85%, 90%, 95%, 100%, or ranges therebetween) sequence identity with SEQ ID NOS: 21-27, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 14, a C-terminal segment comprising at least 70% (e.g., 70%, 75%, 80%, 85%, 90%, 95%, 100%, or ranges therebetween) sequence identity with SEQ ID NO: 21, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 15, a C-terminal segment comprising at least 70% (e.g., 70%, 75%, 80%, 85%, 90%, 95%, 100%, or ranges therebetween) sequence identity with SEQ ID NO: 22, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 16, a C-terminal segment comprising at least 70% (e.g., 70%, 75%, 80%, 85%, 90%, 95%, 100%, or ranges therebetween) sequence identity with SEQ ID NO: 23, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 17, a C-terminal segment comprising at least 70% (e.g., 70%, 75%, 80%, 85%, 90%, 95%, 100%, or ranges therebetween) sequence identity with SEQ ID NO: 24, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 18, a C-terminal segment comprising at least 70% (e.g., 70%, 75%, 80%, 85%, 90%, 95%, 100%, or ranges therebetween) sequence identity with SEQ ID NO: 25, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 19, a C-terminal segment comprising at least 70% (e.g., 70%, 75%, 80%, 85%, 90%, 95%, 100%, or ranges therebetween) sequence identity with SEQ ID NO: 26, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 20, a C-terminal segment comprising at least 70% (e.g., 70%, 75%, 80%, 85%, 90%, 95%, 100%, or ranges therebetween) sequence identity with SEQ ID NO: 27, and an internal segment linking the N-terminal and C-terminal segments, wherein the internal segment is greater than 25 amino acids in length.

In some embodiments, provided herein are compositions comprising a polypeptide having an N-terminal segment comprising at least 70% (e.g., 70%, 75%, 80%, 85%, 90%, 95%, 100%, or ranges therebetween) sequence identity with one of SEQ ID NOS: 81-85, a C-terminal segment comprising at least 70% (e.g., 70%, 75%, 80%, 85%, 90%, 95%, 100%, or ranges therebetween) sequence identity with SEQ ID NOS: 86-90, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 81, a C-terminal segment comprising at least 70% (e.g., 70%, 75%, 80%, 85%, 90%, 95%, 100%, or ranges therebetween) sequence identity with SEQ ID NO: 86, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 82, a C-terminal segment comprising at least 70% (e.g., 70%, 75%, 80%, 85%, 90%, 95%, 100%, or ranges therebetween) sequence identity with SEQ ID NO: 87, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 83, a C-terminal segment comprising at least 70% (e.g., 70%, 75%, 80%, 85%, 90%, 95%, 100%, or ranges therebetween) sequence identity with SEQ ID NO: 88, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 84, a C-terminal segment comprising at least 70% (e.g., 70%, 75%, 80%, 85%, 90%, 95%, 100%, or ranges therebetween) sequence identity with SEQ ID NO: 89, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 85, a C-terminal segment comprising at least 70% (e.g., 70%, 75%, 80%, 85%, 90%, 95%, 100%, or ranges therebetween) sequence identity with SEQ ID NO: 90, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 19, a C-terminal segment comprising at least 70% (e.g., 70%, 75%, 80%, 85%, 90%, 95%, 100%, or ranges therebetween) sequence identity with SEQ ID NO: 26, and an internal segment linking the N-terminal and C-terminal segments, wherein the internal segment is greater than 25 amino acids in length. In some embodiments, the polypeptide comprises a polypeptide having an N-terminal segment comprising at least 70% (e.g., 70%, 75%, 80%, 85%, 90%, 95%, 100%, or ranges therebetween) sequence identity with SEQ ID NO: 20, a C-terminal segment comprising at least 70% (e.g., 70%, 75%, 80%, 85%, 90%, 95%, 100%, or ranges therebetween) sequence identity with SEQ ID NO 27, and an internal segment linking the N-terminal and C-terminal segments, wherein the internal segment is greater than 25 amino acids in length.

In some embodiments, the internal segment is less than 1000 amino acids in length (e.g., 900 amino acids, 800 amino acids, 700 amino acids, 600 amino acids, 500 amino acids, 400 amino acids, 300 amino acids, 200 amino acids, 100 amino acids, or fewer, or ranges therebetween). In some embodiments, the internal segment is a fluorescent or bioluminescent polypeptide capable of emitting energy at a first wavelength. In some embodiments, the internal segment is a component of a bioluminescent complex capable of emitting energy at a first wavelength when contacted by one or more complementary components of the bioluminescent complex and a luminophore. In some embodiments, the internal segment is a binding protein, an enzyme, or an epitope capable of being recognized by a binding protein. In some embodiments, the internal segment comprises at least 70% (e.g., 70%, 75%, 80%, 85%, 90%, 95%, 100%, or ranges therebetween) sequence identity with one of SEQ ID NOS: 28-32 or circularly permuted variates thereof. In some embodiments, the internal segment comprises one of SEQ ID NOS: 28-32 or circularly permuted variates thereof.

In some embodiments, provided herein are compositions comprising a polypeptide having an N-terminal segment comprising at least 70% (e.g., 70%, 75%, 80%, 85%, 90%, 95%, 100%, or ranges therebetween) sequence identity with one of SEQ ID NOS: 6-9, 14-20, and 81-85; a central segment comprising at least 70% (e.g., 70%, 75%, 80%, 85%, 90%, 95%, 100%, or ranges therebetween) sequence identity with one of SEQ ID NOS: 28-32; a C-terminal segment comprising at least 70% (e.g., 70%, 75%, 80%, 85%, 90%, 95%, 100%, or ranges therebetween) sequence identity with one of SEQ ID NOS: 10-13, 21-27, and 86-90; a first internal segment linking the N-terminal and the central segments, and a second internal segment linking the central and C-terminal segments. In some embodiments, provided herein are compositions comprising a polypeptide having an N-terminal segment comprising at least 70% (e.g., 70%, 75%, 80%, 85%, 90%, 95%, 100%, or ranges therebetween) sequence identity with SEQ ID NO: 6, a central segment comprising at least 70% (e.g., 70%, 75%, 80%, 85%, 90%, 95%, 100%, or ranges therebetween) sequence identity with SEQ ID NO 18, a C-terminal segment comprising at least 70% (e.g., 70%, 75%, 80%, 85%, 90%, 95%, 100%, or ranges therebetween) sequence identity with SEQ ID NO 11, a first internal segment linking the N-terminal and the central segments, and a second internal segment linking the central and C-terminal segments. In some embodiments, the first internal segment comprises X₁-X₂₅, wherein each of X₁-X₂₅ is independently selected from any amino acid or absent, wherein at least 5 of X₁-X₂₅ are not absent, and wherein the second internal segment comprises X₂₆-X₅₀, wherein each of X₂₆-X₅₀ is independently selected from any amino acid or absent, wherein at least 5 of X₂₆-X₅₀ are not absent. In some embodiments, the first internal segment comprises X₁-X₂₅, wherein each of X₁-X₂₅ is independently selected from any amino acid or absent, wherein at least 5 of X₁-X₂₅ are not absent, and wherein the second internal segment is greater than 25 amino acids in length. In some embodiments, the second internal segment is a binding protein, fluorescent protein, bioluminescent protein, component of a bioluminescent complex, or enzyme. In some embodiments, the first internal segment and the second internal segment are each greater than 25 amino acids in length. In some embodiments, the first and second internal segments are independently selected from a binding protein, fluorescent protein, bioluminescent protein, component of a bioluminescent complex, and an enzyme.

In some embodiments, provided herein are composition comprising a polypeptide having at least 70% (e.g., 70%, 75%, 80%, 85%, 90%, 95%, 100%, or ranges therebetween) sequence identity with one of SEQ ID NOS: 33-80. In some embodiments, provided herein are methods comprising contacting a polypeptide having at least 70% (e.g., 70%, 75%, 80%, 85%, 90%, 95%, 100%, or ranges therebetween) sequence identity with one of SEQ ID NOS: 33-80 with a luminophore substrate that emits luminescence when contacted by a portion of the polypeptide. In some embodiments, the luminophore substrate is a coelenterazine substrate or derivative thereof (e.g., furimazine). In some embodiments, methods further comprise contacting a composition herein with a substrate of formula (I):

R-linker-A-X,

wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide. In some embodiments, provided herein are systems comprising (a) a polypeptide having at least 70% (e.g., 70%, 75%, 80%, 85%, 90%, 95%, 100%, or ranges therebetween) sequence identity with one of SEQ ID NOS: 33-80; and (b) (i) a luminophore substrate that emits luminescence when contacted by a portion of the polypeptide, and/or (ii) a modified dehalogenase substrate of formula (I):

R-linker-A-X,

wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide. In some embodiments, R is a functional moiety selected from the group consisting of a nucleic acid molecule, an amino acid, a peptide, a receptor protein, a glycoprotein, an antibody, a lipid, a hapten, a receptor ligand, a fluorophore, a photocatalyst, and a toxin.

In some embodiments, provided herein are composition comprising a polypeptide having at least 70% (e.g., 70%, 75%, 80%, 85%, 90%, 95%, 100%, or ranges therebetween) sequence identity with one of SEQ ID NOS: 91-120. In some embodiments, provided herein are methods comprising contacting the polypeptide having at least 70% (e.g., 70%, 75%, 80%, 85%, 90%, 95%, 100%, or ranges therebetween) sequence identity with one of SEQ ID NOS: 91-120 with peptide having at least 70% sequence identity to SEQ ID NO: 30 and a luminophore substrate that emits luminescence when contacted by a complex of the peptide and a portion of the polypeptide. In some embodiments, the luminophore substrate is a coelenterazine substrate or derivative thereof (e.g., furimazine). In some embodiments, methods further comprise contacting the composition with a substrate of formula (I):

R-linker-A-X,

wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide. In some embodiments, provided herein are systems comprising (a) a polypeptide having at least 70% (e.g., 70%, 75%, 80%, 85%, 90%, 95%, 100%, or ranges therebetween) sequence identity with one of SEQ ID NOS: 91-120; (b) a peptide having at least 70% sequence identity with SEQ ID NO: 30; and (c) (i) a luminophore substrate that emits luminescence when contacted by a portion of the polypeptide, and/or (ii) a modified dehalogenase substrate of formula (I):

R-linker-A-X,

wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide. In some embodiments, R is a functional moiety selected from the group consisting of a nucleic acid molecule, an amino acid, a peptide, a receptor protein, a glycoprotein, an antibody, a lipid, a hapten, a receptor ligand, a fluorophore, a photocatalyst, and a toxin.

In some embodiments, provided herein are systems comprising a modified dehalogenase described herein and a substrate of formula (I): R-linker-A-X, wherein A-X is a substrate for a dehalogenase, wherein A is (CH₂)₄₋₂₀ and X is a halide, wherein the linker is a multiatom straight or branched chain including C, N, S, or O, that optionally comprises one or more rings, wherein R is a fluorophore, and wherein X-1-X25 is capable of interacting with the substrate to enhance one or more of substrate binding to the modified dehalogenase, fluorescence intensity of the fluorophore, activation of the fluorophore, and resonance energy transfer to the fluorophore. In some embodiments, the fluorophore is fluorogenic.

In some embodiments, provided herein are methods comprising contacting a modified dehalogenase described herein with a substrate of formula (I):

R-linker-A-X,

wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide.

### BRIEF DESCRIPTION OF THE DRAWINGS

FIG. 1. 3D structure of the HALOTAG modified dehalogenase bound to a chloroalkane ligand, highlighting loop-165 and loop-180.

FIG. 2. TMR ligand labeling activity of loop HaloTag constructs. Each loop received an insertion of 2, 5, or 10 amino acids comprised of Glycine-Serine (Gly-Ser). Constructs were expressed in E. coli and tested in cell lysates, measuring TMR ligand labeling activity in the Total (T) or Soluble (S) fractions of the lysate. Measurements were taken by running samples through SDS-PAGE and scanning the gel for fluorescence.

FIG. 3. JF646 ligand labeling activity and thermostability of loop HaloTag constructs. Each loop received an insertion of 2, 5, or 10 amino acids comprised of Glycine-Serine (Gly-Ser). Constructs were expressed in E. coli and tested in cell lysates following heating at the indicated temperature for 30 minutes by measuring JF646 ligand labeling activity in the lysate. Measurements were taken in a plate-based format measuring the fluorescence of each sample.

FIG. 4A-B. Constructs tested to explore optimal loop extension designs for loop HALOTAG constructs. (A) Design and positioning of 10X-Gly-Ser sequences inserted into loop-165 or loop-180. (B) TMR ligand labeling activity of loop HaloTag constructs. Each loop received insertion of 10 amino acids comprised of Glycine-Serine (Gly-Ser). Constructs were expressed in E. coli and tested in cell lysates, and TMR ligand labeling activity measured in the Total (T) or Soluble (S) fractions of the lysate. Measurements were taken by running samples through SDS-PAGE and scanning the gel for fluorescence.

FIG. 5A-B. TMR and JF646 ligand labeling activity of loop HaloTag library designs. Each loop library design was comprised of insertions at loop-165 or loop-180, with no flanking “noF” residues in the loop commonly used for CDR3 loops in antibodies. The randomized loop sequences tested were 7, 11, or 15 amino acids in length. Constructs were expressed in E. coli and tested in cell lysates by measuring (A) TMR ligand labeling activity using a fluorescence polarization assay or (B) JF646 ligand activation in a fluorescence assay. For comparison, a 6×His-HaloTag (ATG2733) control is included.

FIG. 6A-B. Comparison of loop HaloTag library clones by TMR versus JF646 ligand labeling activity. Each clone was plotted as a single datapoint of its fluorescence intensity with JF646 ligand vs its fluorescence polarization with TMR ligand. (A) Clones highlighted for libraries of 11 or 15 randomized residues in loop-165. (B) Clones highlighted for libraries of 11 or 15 randomized residues in loop-180. For comparison, 6×His-HaloTag (ATG2733) controls are included. Several loop HaloTag variants show HaloTag-like levels of activity with both ligands, whereas others are active only for TMR ligand labeling but not JF646 ligand fluorescence activation.

FIG. 7A-C. Comparison of loop HaloTag library clones by JF646 ligand vs Alexa488 ligand labeling activity. Individual clones with different loop sequences were tested in E. coli lysates for their activity with multiple ligands. (A) Fluorescence intensity of JF646 ligand with loop HaloTag clones shows a range of activities are detected (B) Rate of binding to Alexa488 ligand for loop HaloTag clones shows a different activity pattern, with some clones showing high activity with JF646 but almost no detectable activity with Alexa488 and vice versa. (C) Comparison of loop HaloTag clone activities across multiple ligands. Clones in different quadrants of the graph represent those with more selective substrate specificity.

FIG. 8A-B. Stable sequences enable dual loop HaloTag configurations. Individual clones with different loop sequences at both positions 165 and 180 were tested in E. coli lysates for their activity with TMR. (A) Combinations tested of previously identified sequences at each loop position that resulted in active loop HaloTag clones. (B) Gel electrophoresis of loop HaloTag clones labeled with TMR ligand in E. coli lysates. Protein staining shows consistent amounts of expression across all loop HaloTag clones. Fluorescence detection in the gel shows detectable TMR labeling activity specific to the loop HaloTag clones being tested.

FIG. 9A-D. Characteristics of HaloTag-NLuc fusions and chimeras generated by insertion of circularly-permuted NanoLuc (cpNLuc), circularly-permuted thermostable NanoLuc (cptsNLuc), and circularly-permuted thermostable NanoLuc with a point mutation, F164C (cptsNLuc(F164C)) into loops 165 and 180. Fusion and chimeras were expressed in E. coli, purified, and compared for binding kinetics of a chloroalkane-TMR ligand, brightness of luminescence, and efficiency of intramolecular BRET to a bound TMR ligand. (A) Chimera structures (B) Binding kinetics of 2.5 nM chloroalkane-TMR to 20 nM fusions, and chimeras monitored via fluorescent polarization (C) Total luminescence for 6 nM fusions, and chimeras treated with 20 μM fluorofurimazine (D) Intramolecular BRET efficiencies for 6 nM fusions, and chimeras that were labeled with 5-fold molar excess of chloroalkane-TMR and treated with 20 μM fluorofurimazine.

FIG. 10. Fluorescence emission intensities through BRET to bound fluorophores exhibiting a wide range of overlaps between their excitation spectrum and the bioluminescent reporter emission. Fusions and chimeras expressed in E. coli lysates were labeled with 1 uM fluorescent chloroalkane ligands. Upon treatment with 20 μM fluorofurimazine, emission spectra's (ex=800 nm) were monitored on a SPRK plate reader.

FIG. 11. BRET efficiencies for NLuc-HaloTag vs HT-178-cpNLuc67/68-179 to bound fluorophores exhibiting a wide range of overlaps between their excitation spectrum and the bioluminescent reporter emission. 6 nM purified fusions and chimeras were labeled with 10-fold molar excess fluorescent chloroalkane ligands. Upon treatment with 20 μM fluorofurimazine, emission spectra's (ex=800 nm) were monitored on a SPRK plate reader.

FIG. 12A-D. Binding characteristics of HaloTag-LgBiT fusions, and chimeras generated by insertion of LgBiT and cpLgBiT and cpLgBiT+4 into loop 180. Fusion and chimeras were expressed in E. coli, purified, and compared for binding of a chloroalkane-TMR ligand and complementation affinity with VS-HiBiT (A) Chimera structures (B) Chimeras at equal concentrations were labeled overnight with 5-fold molar excess of TMR ligand, resolved on SDS-PAGE, and scanned for fluorescence (C) Binding kinetics of 2.5 nM chloroalkane-TMR to 20 nM or 160 nM fusions, and chimeras monitored via fluorescent polarization (D) Binding kinetics of 2.5 nM chloroalkane-TMR to 20 nM or 160 nM chimeras following complementation with 10-fold molar excess VS-HiBiT, monitored via fluorescent polarization.

FIG. 13A-B. Luminescence and BRET efficiencies of HaloTag-LgBiT fusions, and chimeras generated by insertion of LgBiT and circularly-permuted LgBiT (cpLgBiT) into loop 180. Fusion and chimeras were expressed in E. coli, purified, and compared for their brightness and efficiency of intramolecular BRET to a bound TMR ligand. (A) Total luminescence for 6 nM fusions and chimeras complemented with 60 nM VS-HiBiT and treated with 20 μM fluorofurimazine (B) Intramolecular BRET efficiencies for 6 nM fusions and chimeras that were complemented with 60 nM VS-HiBiT, labeled with 5-fold molar excess of chloroalkane-TMR, and treated with 20 μM fluorofurimazine.

FIG. 14A-C. Circular permutations of NanoLuc improve donor, acceptor, and BRET when inserted into HaloTag. Sites of circular permutation as indicated in NanoLuc were inserted into loop-180 of HaloTag and expressed in E. coli. Cell lysates containing each construct were labeled with TMR-CA and tested for luminescence and BRET activity upon the addition of fluorofurimazine. The luminescence of (A) donor and (B) acceptor were measured 60 seconds after NanoLuc substrate addition. (C) MilliBRET (mBRET) was calculated as the signal ratio of donor to acceptor (BRET) multiplied by 1,000. The activity of NanoLuc inserted without circular permutation into loop-180 of HaloTag is indicated at far right in black.

FIG. 15A-D. Linker length variations connecting circularly permuted NanoLuc inserted into HaloTag. Circularly permuted NanoLuc at position 67 was inserted into loop-180 of HaloTag with different Glycine-Serine (GS) linker variations and expressed in E. coli. Cell lysates containing each construct were labeled with TMR-CA and tested for luminescence and BRET activity upon the addition of fluorofurimazine. (A) Schematic illustrating the position of linkers inserted into the HaloTag-cpNanoLuc67 chimera. The luminescence of (B) donor and (C) acceptor were measured 60 seconds after NanoLuc substrate addition. (D) MilliBRET (mBRET) was calculated as the signal ratio of donor to acceptor (BRET) multiplied by a factor of 1,000. Constructs are labeled as “HTi_cpN167” representing the insertion of cpNanoLuc67 into HaloTag loop-180. Linker sites are abbreviated “L1”, “L2”, and “L3” according to their position in (A) and the length of the GS-linker indicated as the suffix of their name (i.e., “3” representing a 3 amino acid GS-linker sequence). The activity of NanoLuc inserted without circular permutation into loop-180 of HaloTag is indicated at far right in black.

FIG. 16 A-D. Biochemical characterization of lead HALOTAG-cpNANOLUC chimeras (i.e., circularly permuted NanoLuc inserted into a HaloTag's surface loop) emerging from the screens for alternative circular permutation sites in NanoLuc and flexible linkers that could be incorporated between chimera's components. Chimeras were expressed in E. coli, purified, and compared for binding kinetics of a HaloTag-TMR ligand, brightness, and efficiency of intramolecular BRET to a bound TMR ligand. (A) Structure of the HALOTAG-cpNANOLUC chimeras. (B) Binding kinetics of 2.5 nM HaloTag-TMR ligand to 20 nM chimeras monitored via fluorescent polarization. (C) Total luminescence for 6 nM chimeras treated with 20 μM fluorofurimazine. (D) Intramolecular BRET efficiencies for 6 nM chimeras covalently labeled with HaloTag-TMR ligand and treated with 20 μM fluorofurimazine.

FIG. 17 A-D. Characterization of transiently expressed lead HALOTAG-cpNANOLUC chimeras emerging from the screens for alternative circular permutation sites in NanoLuc and flexible linkers that could be incorporated between chimera's components. Constructs encoding NanoLuc-HaloTag fusion and chimeras were transiently expressed in HeLa cells and evaluated for expression, brightness, and efficiency of intramolecular BRET to a bound TMR ligand. (A) Structure of the HALOTAG-cpNANOLUC chimeras. (B) Expression levels. Lysates from cells labeled with 1 μM HaloTag-TMR ligand were resolved on SDS-PAGE and scanned on a fluorescent imager. Expression levels quantitated using Image J software were normalized to the expression of the NanoLuc-HaloTag fusion. (C) Total luminescence from cells treated with 20 μM fluorofurimazine and further normalized to expression. (D) Intramolecular BRET efficiencies for cells treated with 500 nM HaloTag-TMR ligand.

FIG. 18 A-B. BRET imaging of cells transiently expressing either NanoLuc-HaloTag fusion or lead HALOTAG-cpNANOLUC chimeras emerging from the screens for alternative circular permutation sites in NanoLuc. (A) Images of cells in the presence and absences of a bound HaloTag TMR ligand taken on the Olympus LV200 bioluminescence microscope following treatment with 20 μM fluorofurimazine. Images of donor and acceptor emissions were acquired sequentially using a 460/80 bandpass filter and a 590 nm long-pass filter respectively. (B) BRET ratios for individual cells.

FIG. 19 A-E. Biochemical characterization of chimeras generated by inserting a circularly permuted NanoLuc to HaloTag's loops 180 and 194/195. Chimeras were expressed in E. coli, purified, and compared for binding kinetics of a HaloTag-TMR ligand, brightness, and efficiency of intramolecular BRET to a bound TMR ligand. (A) HaloTag structure with loops and insertion sites annotated. (B) Structure of the HALOTAG-cpNANOLUC chimeras. (C) Binding kinetics of 2.5 nM HaloTag-TMR ligand to 20 nM chimeras monitored via fluorescent polarization. (C) Total luminescence for 6 nM chimeras treated with 20 μM fluorofurimazine. (D) Intramolecular BRET efficiencies for 6 nM chimeras covalently labeled with HaloTag-TMR ligand and treated with 20 μM fluorofurimazine.

FIG. 20 A-F. Biochemical characterization of chimeras genetically fused to dCas12g1 and incorporating additional mutations in the HaloTag's domains. Annotations of the additional mutations are based on a full length non disrupted HaloTag protein. Fusions were expressed in E. coli, purified, and compared for binding kinetics of a chloroalkane-TMR ligand, brightness, and efficiency of intramolecular BRET to a bound TMR ligand. (A) Fusions structures. (B) Binding kinetics of 2.5 nM chloroalkane-TMR to 20 nM fusions monitored via fluorescent polarization. (C) Total luminescence for 6 nM fusions treated with 20 μM fluorofurimazine. (D) Intramolecular BRET efficiencies for 6 nM chimeras covalently labeled with HaloTag-TMR ligand and treated with 20 μM fluorofurimazine. (E) Influence of additional mutations in the HaloTag's domains on binding kinetics of chlHaloTag-TMR ligand. (F) Influence of additional mutations in the HaloTag's domains on brightness and efficiency of intramolecular BRET to a bound TMR ligand.

FIG. 21 A-B. Biochemical characterization of configurations incorporating circularly permuted NanoLucs either as insertions into HaloTag's loop-180 or fusions to a circularly permuted HaloTag. (A) Total luminescence for 6 nM purified proteins treated with 20 μM fluorofurimazine. (B) Intramolecular BRET efficiencies for 6 nM proteins covalently labeled with HaloTag-TMR ligand and treated with 20 μM fluorofurimazine.

FIG. 22 A-I. Biochemical characterization of complementation-based chimeras incorporating flexible linkers and LgBiT+4 circularly permuted at two alternative sites (i.e., 67/68 or 49/50). (A) Structure of the HALOTAG-cpLGBIT chimeras. (B-C) Influence of flexible linkers on binding kinetics of 2.5 nM HaloTag-TMR ligand to 20 nM chimeras, which were complemented with 200 nM VS-HiBiT. (C-D) Influence of flexible linkers on binding affinity to a VS-HiBiT peptide. (E-F) Total luminescence for 6 nM chimeras complemented with 60 nM VS-HiBiT and treated with 20 μM fluorofurimazine. (G-I) Intramolecular BRET efficiencies for 6 nM chimeras complemented with 60 nM VS-HiBiT and covalently labeled with HaloTag-TMR ligand.

FIG. 23 A-G. Characterization of transiently expressed complementation-based chimeras incorporating flexible linkers and LgBiT+4 circularly permuted at two alternative sites (i.e., 67/68 or 49/50). Constructs encoding the chimeras were transfected into genome edited HeLa cells expressing HiBiT-tagged GAPDH. Cells were evaluated for expression, brightness, and efficiency of intramolecular BRET to a bound TMR ligand. (A) Structure of the HALOTAG-cpLGBIT chimeras. (B) Expression levels. Lysates from cells labeled with 1 μM HaloTag-TMR ligand were resolved on SDS-PAGE and scanned on a fluorescent imager. Expression levels quantitated using Image J software were normalized to the expression of HaloTag₁₇₈-cpLgBIT+4 67/68-₁₇₉. (C) Total luminescence from cells treated with 20 μM fluorofurimazine. (D) Intramolecular BRET efficiencies for cells treated with 500 nM HaloTag-TMR ligand.

FIG. 24 A-I. Biochemical characterization of complementation-based chimeras incorporating flexible linkers and LgTrip circularly permuted at two alternative sites (i.e., 67/68 or 49/50). (A) Structure of the HALOTAG-cpLGTRIP chimeras. (B-C) Influence of flexible linkers on binding kinetics of 2.5 nM chloroalkane-TMR to 20 nM chimeras, which were complemented with 200 nM dipeptide (i.e., VS-HiBiT-Trip9). (C-D) Influence of flexible linkers on binding affinity to the dipeptide. (E-F) Total luminescence for 6 nM chimeras complemented with 60 nM dipeptide and treated with 20 μM fluorofurimazine. (G-I) Intramolecular BRET efficiencies for 6 nM chimeras complemented with 60 nM dipeptide and covalently labeled with HaloTag-TMR ligand.

FIG. 25 A-E. Influence of additional LgTrip mutations on biochemical properties of the lead complementation-based chimera HaloTag₁₇₈(L1-3)-cpLgBiT+4-₁₇₉. Annotations of the additional mutations are based on a full length non disrupted NanoLuc protein (A) Structure of the HALOTAG-cpLGBIT chimeras (B) Influence of mutations on binding affinities to the VS-HiBiT peptide. (C) Influence of mutations on brightness and efficiency of intramolecular BRET to a bound TMR ligand for 6 nM chimeras complemented with 60 nM VS-HiBiT. (D-E) Binding kinetics of 2.5 nM HaloTag-TMR ligand to 20 nM or 80 nM chimeras, which were complemented with 200 nM or 800 nM VS-HiBiT, respectively.

FIG. 26 A-C. Influence of additional mutations in the LgBiT domains on biochemical properties the lead complementation-based chimera HaloTag₁₇₈(L1-3)-cpLgBiT+4-₁₇₉. Annotations of the additional mutations are based on a full length non disrupted NanoLuc protein (A) Structure of the HALOTAG-cpLGBIT chimeras. (B) Influence of mutations on binding affinities to the VS-HiBiT peptide. (C) Influence of mutations on brightness and efficiency of intramolecular BRET to a bound TMR ligand for 6 nM chimeras complemented with 60 nM VS-HiBiT.

FIG. 27 A-E. Influence of different L1 linker configurations on biochemical properties of the lead complementation-based chimera HaloTag₁₇₈(L1-3)-cpLgBiT+4-₁₇₉. (A) Structure of the HALOTAG-cpLGBIT chimeras. (B) Influence of mutations on binding affinities to the VS-HiBiT peptide. (C) Influence of mutations on brightness and efficiency of intramolecular BRET to a bound TMR ligand for 6 nM chimeras complemented with 60 nM VS-HiBiT. (D-E) Binding kinetics of 2.5 nM HaloTag-TMR ligand to 20 nM or 80 nM chimeras, which were complemented with 200 nM or 800 nM VS-HiBiT, respectively.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a polypeptide” is a reference to one or more polypeptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-fluorescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., fluorescent intensity of an active fluorophore).

As used herein, when referring to amino acid sequences or positions within an amino acid sequence, the phrase “corresponding to” refers to the relative position of an amino acid residue or an amino acid segment with the sequence being referred to, not the specific identity of the amino acids at that position. For example, a “peptide corresponding to positions 36 through 48 of SEQ ID NO: 1” may comprise less than 100% sequence identity with positions 36 through 48 of SEQ ID NO: 1 (e.g., >70% sequence identity), but within the context of the composition or system being described the peptide relates to those positions.

As used herein, the term “system” refers to multiple components (e.g., devices, compositions, etc.) that find use for a particular purpose. For example, two separate biological molecules, whether present in the same composition or not, may comprise a system if they are useful together for a shared purpose.

As used herein the term “complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to place the elements in the proper proximity for complementarity, to co-localize complementary elements, to lower interaction energy for complementary, to overcome insufficient affinity for one another, etc.

As used herein, the term “complex” refers to an assemblage or aggregate of molecules (e.g., peptides, polypeptides, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides, polypeptides, etc.) is formed under assay conditions such that the complex is thermodynamically favored (e.g., compared to a non-aggregated, or non-complexed, state of its component molecules). As used herein the term “complex,” unless described as otherwise, refers to the assemblage of two or more molecules (e.g., peptides, polypeptides, etc.).

As used herein, the term “fragment” refers to a peptide or polypeptide that results from dissection or “fragmentation” of a larger whole entity (e.g., protein, polypeptide, enzyme, etc.), or a peptide or polypeptide prepared to have the same sequence as such. Therefore, a fragment is a subsequence of the whole entity (e.g., protein, polypeptide, enzyme, etc.) from which it is made and/or designed. A peptide or polypeptide that is not a subsequence of a preexisting whole protein is not a fragment (e.g., not a fragment of a preexisting protein). A peptide or polypeptide that is “not a fragment of a preexisting protein” is an amino acid chain that is not a subsequence of a protein (e.g., natural or synthetic) that was in physical existence prior to design and/or synthesis of the peptide or polypeptide. A fragment of a hydrolase or dehalogenase, as used herein, is a sequence which is less than the full-length sequence, but which alone cannot form a substrate binding site, and/or has substantially reduced or no substrate binding activity but which, in close proximity to a second fragment of a hydrolase or dehalogenase, exhibits substantially increased substrate binding activity. In one embodiment, a fragment of a hydrolase or dehalogenase is at least 5, e.g., at least 10, at least 20, at least 30, at least 40, or at least 50, contiguous residues of a wild-type hydrolase or a mutated hydrolase, or a sequence with at least 70% sequence identity thereto, and may not necessarily include the N-terminal or C-terminal residue or N-terminal or C-terminal sequences of the corresponding full length protein.

As used herein, the term “subsequence” refers to peptide or polypeptide that has 100% sequence identify with a portion of another, larger peptide, or polypeptide. The subsequence is a perfect sequence match for a portion of the larger amino acid chain.

The term “amino acid” refers to natural amino acids, unnatural amino acids, and amino acid analogs, all in their D and L stereoisomers, unless otherwise indicated, if their structures allow such stereoisomeric forms.

The term “proteinogenic amino acids” refers to the 20 amino acids coded for in the human genetic code, and includes alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V). Selenocysteine and pyrrolysine may also be considered proteinogenic amino acids

The term “non-proteinogenic amino acid” refers to an amino acid that is not naturally-encoded or found in the genetic code of any organism, and is not incorporated biosynthetically into proteins during translation. Non-proteinogenic amino acids may be “unnatural amino acids” (amino acids that do not occur in nature) or “naturally-occurring non-proteinogenic amino acids” (e.g., norvaline, ornithine, homocysteine, etc.). Examples of non-proteinogenic amino acids include, but are not limited to, azetidinecarboxylic acid, 2-aminoadipic acid, 3-aminoadipic acid, beta-alanine, naphthylalanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-alkylglycine including N-methylglycine, N-methylisoleucine, N-alkylpentylglycine including N-methylpentylglycine. N-methylvaline, naphthylalanine, norvaline, norleucine (“Norleu”), octylglycine, ornithine, pentylglycine, pipecolic acid, thioproline, homolysine, and homoarginine. Non-proteinogenic also include D-amino acid forms of any of the amino acids herein, as well as non-alpha amino acid forms of any of the amino acids herein (beta-amino acids, gamma-amino acids, delta-amino acids, etc.), all of which are in the scope herein and may be included in peptides herein.

The term “amino acid analog” refers to an amino acid (e.g., natural or unnatural, proteinogenic or non-proteinogenic) where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide, and S-(carboxymethyl)-cysteine sulfone.

As used herein, unless otherwise specified, the terms “peptide” and “polypeptide” refer to polymer compounds of two or more amino acids joined through the main chain by peptide amide bonds (—C(O)NH—). The term “peptide” typically refers to short amino acid polymers (e.g., chains having fewer than 30 amino acids), whereas the term “polypeptide” typically refers to longer amino acid polymers (e.g., chains having more than 30 amino acids). As used herein, the term “artificial” refers to compositions and systems that are designed or prepared by man and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any peptide/polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “wild-type,” refers to a gene or gene product (e.g., protein, polypeptide, peptide, etc.) that has the characteristics (e.g., sequence) of that gene or gene product isolated from a naturally occurring source, and is most frequently observed in a population. In contrast, the term “mutant” or “variant” refers to a gene or gene product that displays modifications in sequence when compared to the wild-type gene or gene product. It is noted that “naturally-occurring variants” are genes or gene products that occur in nature, but have altered sequences when compared to the wild-type gene or gene product; they are not the most commonly occurring sequence. “Artificial variants” are genes or gene products that have altered sequences when compared to the wild-type gene or gene product and do not occur in nature. Variant genes or gene products may be naturally occurring sequences that are present in nature, but not the most common variant of the gene or gene product, or “synthetic,” produced by human or experimental intervention.

As used herein, the term “physiological conditions” encompasses any conditions compatible with living cells, e.g., predominantly aqueous conditions of a temperature, pH, salinity, chemical makeup, etc. that are compatible with living cells.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum, and the like. Sample may also refer to cell lysates or purified forms of the enzymes, peptides, and/or polypeptides described herein. Cell lysates may include cells that have been lysed with a lysing agent or lysates such as rabbit reticulocyte or wheat germ lysates. Sample may also include cell-free expression systems. Environmental samples include environmental material such as surface matter, soil, water, crystals, and industrial samples. Such examples are not however to be construed as limiting the sample types applicable to the present invention.

As used herein, the terms “fusion,” “fusion polypeptide,” and “fusion protein” refer to a chimeric protein containing a first protein or polypeptide of interest (e.g., substantially non-luminescent peptide) joined to a second different peptide, polypeptide, or protein (e.g., interaction element).

As used herein, the terms “conjugated” and “conjugation” refer to the covalent attachment of two molecular entities (e.g., post-synthesis and/or during synthetic production). The attachment of a peptide or small molecule tag to a protein or small molecule, chemically (e.g., “chemically” conjugated) or enzymatically, is an example of conjugation.

As used herein, the term “dehalogenase” refers to an enzyme that catalyzes the removal of a halogen atom from a substrate. The term “haloalkane dehalogenase” refers to an enzyme that catalyzes the removal of a halogen from a haloalkane substrate to produce a alcohol and a halide. Dehalogenases and haloalkyl dehalogenases belong to the hydrolase enzyme family, and may be referred to herein or elsewhere as such.

As used herein, the term “modified dehalogenase” refers to a dehalogenase variant (artificial variant) that has mutations that prevent the release of the substrate from the protein following removal of the halogen, resulting in a covalent bond between the substrate and the modified dehalogenase. The HALOTAG system (Promega) is a commercially available modified dehalogenase and substrate system.

As used herein, the term “circularly-permuted” (“cp”) refers to a polypeptide in which the N- and C-termini have been joined together, either directly or through a linker, to produce a circular polypeptide, and then the circular polypeptide is opened at a location other than between the N- and C-termini to produce a new linear polypeptide with termini different from the termini in the original polypeptide. The location at which the circular polypeptide is opened is referred to herein as the “cp site.” Circular permutants include those polypeptides with sequences and structures that are equivalent to a polypeptide that has been circularized and then opened. Thus, a cp polypeptide may be synthesized de novo as a linear molecule and never go through a circularization and opening step. The preparation of circularly permutated derivatives is described in WO95/27732; incorporated by reference in its entirety.

As used herein, the term “luminescence” refers to the emission of light by a substance as a result of a chemical reaction (“chemiluminescence”) or an enzymatic reaction (“bioluminescence”).

As used herein, the term “bioluminescence” refers to production and emission of light by a reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “luminophore” refers to a chemical moiety or compound that can be placed in an excited electronic state (e.g., by a chemical or enzymatic reaction) and emits light as it returns to its electronic ground state.

As used herein, the term “imidazopyrazine luminophore” refers to a genus of luminophores including “native coelenterazine” as well as synthetic (e.g., derivative or variant) and natural analogs thereof, including furimazine, furimazine analogs (e.g., fluorofurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference herein in their entireties.

As used herein, the term “coelenterazine” refers to the naturally-occurring (“native”) imidazopyrazine of the structure:

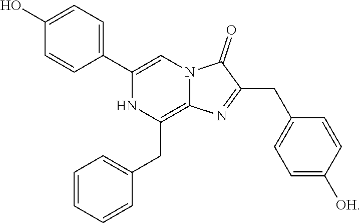 [SMILES: O=c1c(Cc2ccc(O)cc2)nc2c(Cc3ccccc3)[nH]c(-c3ccc(O)cc3)cn1-2]

As used herein, the term “furimazine” refers to the coelenterazine derivative of the structure:

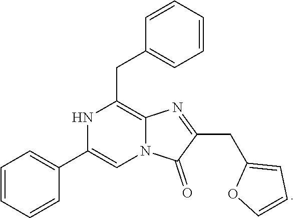 [SMILES: O=c1c(Cc2ccco2)nc2c(Cc3ccccc3)[nH]c(-c3ccccc3)cn1-2]

As used herein, the term “fluorofurimazine” refers to the furimazine derivative of the structure:

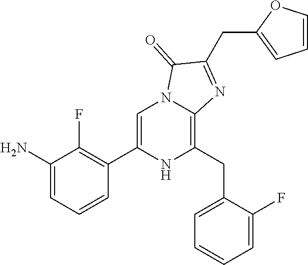 [SMILES: Nc1cccc(-c2cn3c(=O)c(Cc4ccco4)nc-3c(Cc3ccccc3F)[nH]2)c1F]

(U.S. application Ser. No. 16/548,214; incorporated by reference in its entirety).

As used herein, the term “bioluminescence resonance energy transfer” (“BRET”) refers to the distance-dependent interaction in which energy is transferred from a donor bioluminescent protein/complex and substrate to an acceptor molecule without emission of a photon. The efficiency of BRET is dependent on the inverse sixth power of the intermolecular separation, making it useful over distances comparable with the dimensions of biological macromolecules (e.g., within 30-80 Å, depending on the degree of spectral overlap).

As used herein, the term “an Oplophorus luciferase” (“an OgLuc”) refers to a luminescent polypeptide having significant sequence identity, structural conservation, and/or the functional activity of the luciferase produce by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 28 (NANOLUC), which comprises 10 β strands (β1, β2, β3, β4, β5, β6, β7, β8, β9, β10) and utilize substrates such as coelenterazine or a coelenterazine derivative or analog to produce luminescence.

### DETAILED DESCRIPTION

Provided herein are modified dehalogenases that have extended surface loop regions that provide a location for internal fusion insertions and modulate binding interaction, energy transfer, and activation of environmentally-sensitive chemistries.

The development of new fluorophores and fluorogenic dyes (such as the JANELIA FLUOR dyes) for use with chloroalkanes (CA) highlights the recent interest in HALOTAG for fluorescence detection in cell imaging applications. The advantages of such dyes in brightness, photostability, sensitivity, and far-red spectral detection over conventional tools, such as widely-used fluorescent proteins, is particularly apparent in challenging or highly sensitive imaging applications in endogenous biology. As chloroalkane conjugates, they can take advantage of the self-labeling activity of HALOTAG to measure protein abundance and localization in a target-specific manner through genetic fusion.

Recent evidence supports a model for activation of rhodamine-based fluorogenic dyes attached to chloroalkanes through physical interactions with the surface of HALOTAG after binding. This interaction changes the equilibrium of the dye away from its non-fluorescent lactone state toward its fluorescent zwitterionic state. Much effort has been put into chemical modification of the dye scaffold itself to enhance this effect through changes that modulate the lactone-zwitterionic structural equilibrium. However, chemical modification of the dye structure pushing the equilibrium toward the zwitterionic state to enhance fluorescence also tends to make the ligands less cell permeable, and similarly, those favoring the lactone state enhance permeability at the cost of fluorescence yield. Relative to chemical modifications of fluorogenic dyes, modifications to HALOTAG itself have been less well explored. Point mutantions in HALOTAG have been shown to enhance fluorogenicity by making protein:dye interactions more favorable for fluorescence (Frei et al. Engineered HaloTag variants for fluorescence lifetime multiplexing. Nature Methods volume 19, pages 65-70 (2022); incorporated by reference in its entirety).

Experiments were conducted during development of embodiments herein to improve activation of chemistries dyes, such as fluorogenic dyes, on the surface of the modified dehalogenase, HALOTAG. It was reasoned that one ideal solution would include an engineered protein surface for optimal interactions that improve fluorescence activation upon binding. While point mutations in surface residues have been shown to be one such solution, they are intrinsically limited in the positioning and sequence availability of the native HALOTAG protein scaffold. Efforts were undertaken to engineer extended loops into the HALOTAG structure in regions proximal to dye interaction sites, for example, in order to provide additional interaction surface area and significantly increase available sequence space for optimization. In addition to increased interaction surface, this solution also provides new binding mechanisms between the dye and protein that are only achievable through the conformations of the extended loops, thereby providing entirely new chemical activation schemes. The range of activatable chemistries is thus significantly increased in a manner proportional to the vastly new protein sequence space and structure available in the extended loop regions. The utility of the extended loops is not limited to the activation of dyes and/or improved interactions with substrates, and such activation/interactions are not necessary to practice the invention.

The extended HALOTAG loops find use in the activation of fluorogenic dyes, but can also be extended to a wide range of environmentally-sensitive, CA-conjugated chemistries that are activated by an optimized binding surface or pocket formed through engineered loop sequences on the surface of HALOTAG. Thus, engineered “loop HALOTAG” variants may be tailored for activation of environmentally-sensitive chemistries in a robust and orthogonal manner following binding. For example, the extended loops find use in enhancing activation of dyes/chemistries via BRET, and the extended loops are utilized to further engineer chimeras of HALOTAG with bioluminescent reporters to improve the efficiency of BRET-based activation through more favorable proximity/geometry for BRET between the bioluminescent reporter and the bound ligand. This is especially critical when the spectral overlap between the emission of the bioluminescent reporter and the excitation of the ligand is significantly limited. One downstream application of this improved efficiency is the use of a bioluminescent light source as the activator of downstream chemistries.

Embodiments herein are not limited to enhancing interactions between the loops and ligands or interaction partners. In some embodiments, the regions identified herein (e.g., loop 165, loop 180, loop 194/195) find use as a location for insertion of peptides or polypeptides into the HALOTAG sequence. For example, the extended loops also provide a location for the insertion of larger polypeptides, such as proteins or enzymes, into HALOTAG for optimal positioning or geometry close to the bound ligand. In some embodiments, chimeras formed at internal loop sites increase the efficiency of energy transfer between the inserted protein and the HALOTAG ligand through BRET or FRET, particularly when the spectral overlap between the emission of the inserted reporter and the excitation of the HALOTAG ligand is significantly limited. For example, it has been demonstrated that a circularly permuted NANOLUC luciferase (cpNL) increases the efficiency of BRET with a fluorescent HALOTAG ligand when inserted at a position within the HALOTAG lid domain (Hiblot, J., et al. (2017) Angew Chem Int Ed Engl 56(46): 14556-14560; incorporated by reference in its entirety). In some embodiments, this strategy provides a solution for similarly increasing FRET efficiency, for example, when a fluorescent protein (e.g., GFP, RFP, etc.) is inserted into the loop regions disclosed herein proximal to a fluorescent HALOTAG ligand.

Evidence from structural analysis of HALOTAG bound to fluorescent and fluorogenic ligands alongside mutation studies has supported a model of fluorescence activation of rhodamine-based dyes through surface contacts between HALOTAG and the dye moiety. In particular, Helix 8 of HALOTAG (residues ˜167-176) is positioned in direct contact with the dye in several structures. Experiments conducted during development of embodiments herein have demonstrated that deletions, circular permutations, and/or splits within or proximal to this region of HALOTAG eliminate fluorogenic activation of the ligand. It was therefore deemed reasonable that point mutations in the surface of HALOTAG could make these interactions more favorable and ultimately lead to increase fluorogenic activation. However, introducing point mutations into the binding surface of HALOTAG, although a successful strategy thus far, would ultimately be limited by the position of existing residues in the protein scaffold and risk perturbing the fold of the protein as they also contribute to its native structure. In addition, it is likely that only a small number of residues have the potential for optimization due to their proximity to the dye moiety of the ligand, limiting the utility of this approach.

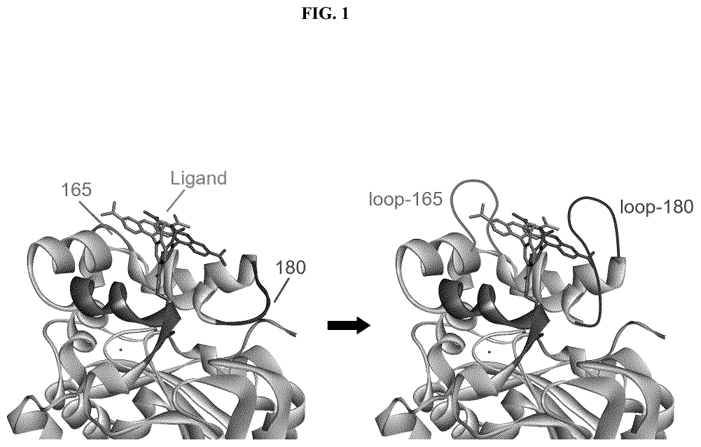

Experiments were conducted during development of embodiments herein to introduce additional protein sequence into this critical region of HALOTAG in order to increase the binding surface and configurations available for optimization of interactions. The loop regions flanking Helix 8 in HALOTAG were targeted for modification, because: (1) loops are generally more tolerant to insertion, modification, or deletion without significantly disrupting the fold or function of proteins, and (2) they are in close proximity to the bound dye in the crystal structure and might position newly inserted residues within a distance capable of forming interactions. The two loops flanking Helix 8, herein denoted as loop-165 (residues 164-166) and loop-180 (residues 177-182), are both in the lid subdomain of HALOTAG that comprises the majority of the ligand binding tunnel and surface-exposed tunnel opening (FIG. 1). Empirical steps were taken to engineer extended loop regions into HALOTAG at these positions. Optimal sites were identified for insertion of residues in loop-165 or loop-180. Preliminary screening was performed to identify several sequence insertions of 7-15 residues in length that result in loop HALOTAG variants with unique activity profiles, demonstrating the utility of this concept. To further demonstrate the feasibility of introducing relatively large sequences (e.g., bioluminescent reporters), experiments were conducted during development of embodiments herein to insert bioluminescent reporters into the extended loops. The resulting chimeras were used for activation of bound dyes via an intrinsic intramolecular bioluminescence resonance energy transfer mechanism.

Extended surface loops provide various benefits that are expected to improve and/or expand upon the capabilities and applications of HALOTAG. First, much like the complementary determining region (CDR) loops of antibodies, the extended surface loops can adopt diverse conformations comprised of different amino acid sequences that make them suitable for highly divergent yet specific binding modes. There are examples of antibodies and other binding scaffolds (e.g., DARPINS, scFVs, and Nanobodies) that have been engineered to bind small molecules, including fluorogenic dyes in a manner that increases their fluorescence. Specific recognition of small molecules by antibodies is not trivial to engineer, however, and structural and biophysical analysis has revealed that binding is commonly achieved through dimerization of the antibody around the small molecule target, essentially creating a binding pocket between monomers. In some embodiments, the advantages of molecular recognition through extended loops in HALOTAG overcomes this challenge since binding is already achieved through its robust interaction and self-labeling activity with the CA in a monomeric complex. In this scenario, covalent attachment of the CA to HALOTAG positions the conjugated small molecule cargo on its surface, enabling residues in the proximal extended loop regions to interact, thereby reducing the engineering burden required for activation by removing the need to also engineer robust and specific ligand affinity.

Molecular recognition by extended surface loops in HaloTag is not limited to purposes of activating CA conjugates. In some embodiments, the extended loops interact with intermolecular binding partners, such as other proteins, akin to antibody recognition, and target HALOTAG (and its bound CA ligands) to specific targets inside cells or as part of diagnostic assays, for example. These configurations of extended loop HALOTAG retain many of the advantages of antibodies, but also include the capability to genetically encode the construct and deliver a ligand of interest as a CA conjugate in proximity to the protein target as well. Beyond molecular recognition, the utility provided by the extended HALOTAG loops enables new conformations and geometries of chimera proteins inserted within the loops. For example, larger polypeptides can be engineered into favorable distances and geometries, enabling more efficient energy transfer between the inserted polypeptide (such as a bioluminescent enzyme) and the bound HALOTAG ligand. This is particularly important when there is limited spectral overlap between the emission of the bioluminescent reporter and the excitation of the HaloTag ligand, where distance and geometry within the chimera is critical for energy transfer.

The additional capabilities of an extended-loop HALOTAG design confer capacity for molecular interactions that extend the useful applications of HALOTAG. For example:
1. Extended loops enable increased fluorescence (or a range of fluorescence activations) of HALOTAG fluorogenic ligands, such as those currently commercially available (i.e., CA-Janelia Fluor dyes: Promega corp., Madison, WI). Increased fluorescence is realized as either signal intensity or fluorescence lifetime in the presence of engineered extended loops in HALOTAG. Differences in fluorescence lifetime have been shown to be valuable for HALOTAG-9/10/11 multiplexing in fluorescence imaging (Frei, M. et al (2022). Nature Methods. (19) 65-70; incorporated by reference in its entirety). An additional category of new uses for existing HaloTag fluorescent/fluorogenic ligands includes BRET- and FRET-based applications, where chimeras are created by using these extended loops as insertion sites to create chimeras with bioluminescent or fluorescent proteins. For BRET, several applications include a) BRET as the means to tune the emission of NANOLUC-based bioluminescent reporters for cell/animal imaging; b) sorting HIBIT-edited cells, where labeling is dependent on complementation with LGBIT; c) BRET-triggered activation of light sensitivity molecules including catalysts; and d) BRET-triggered bioluminolysis.
2. HALOTAG fluorogenic ligand systems. Provided herein are extended loop HALOTAG variants with CA-fluorogenic dyes capable of greater fluorescence yield or signal-to-background upon activation. In some embodiments, CA-fluorogenic dyes do not have significant activation with unmodified HALOTAG. For example, certain Janelia Fluor dyes, for example, with a stronger natural preference toward the non-fluorescent lactone state (which are more cell permeable) but are more difficult to transition to the fluorescent zwitterionic state without the additional stabilizing molecular interactions provided by the optimized extended surface in the extended loop modified dehalogenases herein. Such improved systems find use in, for example, cell imaging, where the simultaneous reduction in background signal of the non-fluorescent free ligand and greater potential activation of the bound ligand create overall better signal-to-background ratios for imaging on top of better cell/tissue permeability of dyes in the lactone state.
3. Chemistries that are specifically compatible/activatable with engineered loop modified dehalogenase variants. Beyond fluorogenic dyes, there are a number of commercially valuable ligands such as catalysts, biosensors, and proximity labels that find use as CA conjugates and undergo stabilization of their structural transitions by interactions with extended-loop modified dehalogenases. Such systems are configured to allow a tunable range of responses. For example, BAPTA-CA ligands have been shown to be intracellular indicators that undergo a conformational change upon chelating Ca2+ ions to increase their fluorescence, making them sensitive synthetic biosensors for Ca2+ flux inside living cells. The Ca2+ response of the BAPTA-CA ligands can be chemically tuned across a range of affinities but typically at the expense of quantum yield. An optimized extended-loop modified dehalogenase provides a BAPTA-CA response to physiologically-relevant Ca2+ level with higher quantum yield in a manner that cannot be achieved through synthetic chemical modification of the ligand alone. As another example, the calcium indicating/chelating moiety alters the fluorogenicity of the CA-dye in a manner that is tunable for affinity and color, this was particularly valuable for providing a Calcium indicator in the red (˜650 nm) range of detection (Mertes et al. J Am. Chem. Soc. 2022, 144, 15, 6928-6935; incorporated by reference in its entirety).
4. Affinity reagents based on extended-loop modified dehalogenases. Extended-loop modified dehalogenases that recognize other molecular targets like proteins provides a wide range of utilities, such as standalone affinity reagents, purification/enrichment systems, diagnostics, imaging tools, or genetically-encodable intracellular bioassays. These systems all benefit from the localization of CA-ligands upon binding of an extended-loop modified dehalogenase to its target.

The modified dehalogenases, systems, and methods herein are not limited by the specific utilities and uses described herein, and an understanding of the utility or use of the modified dehalogenase is not necessary to practice the invention. Any embodiment comprising a modified dehalogenase with an amino acid sequence inserted internally at one of the positions described herein is within the scope herein. An enhanced capacity to activate a substrate or provide an interaction is not necessary to a modified dehalogenase with an internal insertion to be within the scope herein.

### I. Modified Dehalogenases

In some embodiments, provided herein are modified dehalogenases with internal insertions. In some embodiments, the modified dehalogenase is the commercially-available HALOTAG protein (SEQ ID NO: 1), or a variant thereof (e.g., >70% sequence identity). HALOTAG is a 297-residue self-labeling polypeptide (33 kDa) derived from a bacterial hydrolase (dehalogenase) enzyme, which has modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group.

The HALOTAG polypeptide is a hydrolase with a genetically modified active site, which specifically binds to the haloalkane ligand chloroalkane linker with an enhanced and increased rate of ligand binding (Pries et at The Journal of Biological Chemistry. 270(18):10405-11; incorporated by reference in its entirety). The reaction that forms the bond between the protein tag and chloroalkane linker is fast and essentially irreversible under physiological conditions (Waugh DS (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety).

HALOTAG fusion proteins can be expressed using standard recombinant protein expression techniques (Adams et al. (May 2002) Journal of the American Chemical Society. 124(21):6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5):571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen DB (April 2004). Current Opinion in Chemical Biology. 8(2):150-9; incorporated by reference in its entirety).

Various HALOTAG ligands, functional groups, fusions, assays, modifications, uses, etc., are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

As described herein, embodiments are not limited to the HALOTAG sequence. In some embodiments, provided herein are split modified dehalogenases that differ in sequence from SEQ ID NO: 1. In some embodiments, provided herein are split dehalogenases that lack the mutation(s) (e.g., 272 and/or 106) that produce covalent bonding to the haloalkane substrate. Such sp dehalogenases are true enzymes capable of substrate turnover, but otherwise comprising the sequences and characteristics of the embodiments described herein.

In some embodiments, provided herein are polypeptides and fusions derived from a modified dehalogenase sequence of SEQ ID NO: 1:

MAEIGTGFPFDPHYVEVLGERMHYVDVGPRDGTPVLFLHGNPTSS
YVWRNIIPHVAPTHRCIAPDLIGMGKSDKPDLGYFFDDHVREMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLNPVDREPLWRFPNELPIAGEPANIVALVEEY
MDWLHQSPVPKLLFWGTPGVLIPPAEAARLAKSLPNCKAVDIGPG
LNLLQEDNPDLIGSEIARWLSTLEISG.

In some embodiments, modified dehalogenase polypeptides herein comprise at least 70% sequence identity with all or a portion of SEQ ID NO: 1 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, polypeptides herein comprise 100% sequence identity with all or a portion of SEQ ID NO: 1. In some embodiments, polypeptides herein comprise at least 70% sequence similarity with all or a portion of SEQ ID NO: 1 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, polypeptides herein comprise 100% sequence similarity with all or a portion of SEQ ID NO: 1.

In some embodiments, provided herein are modified dehalogenase polypeptides comprising at least 70% sequence identity (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) with SEQ ID NO: 1, but with an insertion of an extended loop sequence (e.g., 1-25 amino acids in length) or a peptide or polypeptide at a position or sequence within he SEQ ID NO: 1 sequence (e.g., replacing loop 165, replacing loop 180, replacing loop 194/195, following position 165, following position 180, following position 194, etc.).

In some embodiments, provided herein are modified dehalogenase polypeptides comprising an insertion of up to 25 amino acids in length (e.g., 1, 2, 3, 4, 5, 6, 7, 8, 9, 10, 11, 12, 13, 14, 15, 16, 17, 18, 19, 20, 21, 22, 23, 24, 25 amino acids, or ranges therebetween) within loop 165 of SEQ ID NO: 1. In some embodiments, provided herein are polypeptides comprising at least 70% sequence identity with all or a portion of SEQ ID NO: 2 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, polypeptides herein comprise 100% sequence identity with all or a portion of SEQ ID NO: 2. In some embodiments, polypeptides herein comprise at least 70% sequence similarity with all or a portion of SEQ ID NO: 2 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, polypeptides herein comprise 100% sequence similarity with all or a portion of SEQ ID NO: 2.

In some embodiments, provided herein are modified dehalogenase polypeptides comprising an insertion of up to 25 amino acids in length (e.g., 1, 2, 3, 4, 5, 6, 7, 8, 9, 10, 11, 12, 13, 14, 15, 16, 17, 18, 19, 20, 21, 22, 23, 24, 25 amino acids, or ranges therebetween) at the position corresponding to the position following position 165 of SEQ ID NO: 1. In some embodiments, provided herein are polypeptides comprising at least 70% sequence identity with all or a portion of SEQ ID NO: 3 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, polypeptides herein comprise 100% sequence identity with all or a portion of SEQ ID NO: 3. In some embodiments, polypeptides herein comprise at least 70% sequence similarity with all or a portion of SEQ ID NO: 3 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, polypeptides herein comprise 100% sequence similarity with all or a portion of SEQ ID NO: 3.

In some embodiments, provided herein are modified dehalogenase polypeptides comprising an insertion of up to 25 amino acids in length (e.g., 1, 2, 3, 4, 5, 6, 7, 8, 9, 10, 11, 12, 13, 14, 15, 16, 17, 18, 19, 20, 21, 22, 23, 24, 25 amino acids, or ranges therebetween) within loop 180 of SEQ ID NO: 1. In some embodiments, provided herein are polypeptides comprising at least 70% sequence identity with all or a portion of SEQ ID NO: 4 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, polypeptides herein comprise 100% sequence identity with all or a portion of SEQ ID NO: 4. In some embodiments, polypeptides herein comprise at least 70% sequence similarity with all or a portion of SEQ ID NO: 4 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, polypeptides herein comprise 100% sequence similarity with all or a portion of SEQ ID NO: 4.

In some embodiments, provided herein are modified dehalogenase polypeptides comprising an insertion of up to 25 amino acids in length (e.g., 1, 2, 3, 4, 5, 6, 7, 8, 9, 10, 11, 12, 13, 14, 15, 16, 17, 18, 19, 20, 21, 22, 23, 24, 25 amino acids, or ranges therebetween) at the position corresponding to the position following position 180 of SEQ ID NO: 1. In some embodiments, provided herein are polypeptides comprising at least 70% sequence identity with all or a portion of SEQ ID NO: 5 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity). In some embodiments, polypeptides herein comprise 100% sequence identity with all or a portion of SEQ ID NO: 5. In some embodiments, polypeptides herein comprise at least 70% sequence similarity with all or a portion of SEQ ID NO: 5 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, polypeptides herein comprise 100% sequence similarity with all or a portion of SEQ ID NO: 5.

In some embodiments, provided herein are modified dehalogenase polypeptides comprising a peptide or polypeptide (e.g., protein) inserted at an internal location (e.g., replacing loop 165, replacing loop 180, replacing loop 194/195, following position 165, following position 180, following position 194, etc.). In some embodiments, the inserted sequence is 1, 2, 5, 10, 20, 50, 100, 150, 200, 250, 300, 400, 500, or more amino acids in length. In some embodiments, the inserted sequence and the modified dehalogenase each retain all or a portion (e.g., >10%, >25%, >50%, >75%, >90%) of their activity and/or functionality (e.g., substrate binding capacity).

In some embodiments, provided herein are modified dehalogenase polypeptides comprising a peptide or polypeptide insertion within a loop corresponding to loop 165 of SEQ ID NO: 1. In some embodiments, provided herein are modified dehalogenase polypeptides comprising a first sequence having at least 70% sequence identity with all or a portion of one of SEQ ID NOS: 6-9 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of one of SEQ ID NOS: 10-13 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence.

In some embodiments, provided herein are modified dehalogenase polypeptides comprising a first sequence having at least 70% sequence identity with all or a portion of SEQ ID NO: 6 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 10 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 6. In some embodiments, the first sequence comprises at least 70% sequence similarity with all or a portion of SEQ ID NO: 6 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 6. In some embodiments, the second sequence comprises 100% sequence identity with all or a portion of SEQ ID NO: 10. In some embodiments, the second sequence comprises at least 70% sequence similarity with all or a portion of SEQ ID NO: 10 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 10.

In some embodiments, provided herein are modified dehalogenase polypeptides comprising a first sequence having at least 70% sequence identity with all or a portion of SEQ ID NO: 7 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 11 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 7. In some embodiments, the first sequence comprises at least 70% sequence similarity with all or a portion of SEQ ID NO: 7 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 7. In some embodiments, the second sequence comprises 100% sequence identity with all or a portion of SEQ ID NO: 11. In some embodiments, the second sequence comprises at least 70% sequence similarity with all or a portion of SEQ ID NO: 11 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 11.

In some embodiments, provided herein are modified dehalogenase polypeptides comprising a first sequence having at least 70% sequence identity with all or a portion of SEQ ID NO: 8 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 12 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 8. In some embodiments, the first sequence comprises at least 70% sequence similarity with all or a portion of SEQ ID NO: 8 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 8. In some embodiments, the second sequence comprises 100% sequence identity with all or a portion of SEQ ID NO: 12. In some embodiments, the second sequence comprises at least 70% sequence similarity with all or a portion of SEQ ID NO: 12 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 12.

In some embodiments, provided herein are modified dehalogenase polypeptides comprising a first sequence having at least 70% sequence identity with all or a portion of SEQ ID NO: 9 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 13 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 9. In some embodiments, the first sequence comprises at least 70% sequence similarity with all or a portion of SEQ ID NO: 9 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 9. In some embodiments, the second sequence comprises 100% sequence identity with all or a portion of SEQ ID NO: 13. In some embodiments, the second sequence comprises at least 70% sequence similarity with all or a portion of SEQ ID NO: 13 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 13.

In some embodiments, provided herein are modified dehalogenase polypeptides comprising a peptide or polypeptide insertion within a loop corresponding to loop 180 of SEQ ID NO: 1. In some embodiments, provided herein are modified dehalogenase polypeptides comprising a first sequence having at least 70% sequence identity with all or a portion of one of SEQ ID NOS: 14-20 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of one of SEQ ID NOS: 21-27 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence.

In some embodiments, provided herein are modified dehalogenase polypeptides comprising a first sequence having at least 70% sequence identity with all or a portion of SEQ ID NO: 14 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 21 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 14. In some embodiments, the first sequence comprises at least 70% sequence similarity with all or a portion of SEQ ID NO: 14 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 14. In some embodiments, the second sequence comprises 100% sequence identity with all or a portion of SEQ ID NO: 21. In some embodiments, the second sequence comprises at least 70% sequence similarity with all or a portion of SEQ ID NO: 21 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 21.

In some embodiments, provided herein are modified dehalogenase polypeptides comprising a first sequence having at least 70% sequence identity with all or a portion of SEQ ID NO: 15 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 22 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 15. In some embodiments, the first sequence comprises at least 70% sequence similarity with all or a portion of SEQ ID NO: 15 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 15. In some embodiments, the second sequence comprises 100% sequence identity with all or a portion of SEQ ID NO: 22. In some embodiments, the second sequence comprises at least 70% sequence similarity with all or a portion of SEQ ID NO: 22 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 22.

In some embodiments, provided herein are modified dehalogenase polypeptides comprising a first sequence having at least 70% sequence identity with all or a portion of SEQ ID NO: 16 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 23 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 16. In some embodiments, the first sequence comprises at least 70% sequence similarity with all or a portion of SEQ ID NO: 16 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 16. In some embodiments, the second sequence comprises 100% sequence identity with all or a portion of SEQ ID NO: 23. In some embodiments, the second sequence comprises at least 70% sequence similarity with all or a portion of SEQ ID NO: 23 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 23.

In some embodiments, provided herein are modified dehalogenase polypeptides comprising a first sequence having at least 70% sequence identity with all or a portion of SEQ ID NO: 17 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 24 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 17. In some embodiments, the first sequence comprises at least 70% sequence similarity with all or a portion of SEQ ID NO: 17 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 17. In some embodiments, the second sequence comprises 100% sequence identity with all or a portion of SEQ ID NO: 24. In some embodiments, the second sequence comprises at least 70% sequence similarity with all or a portion of SEQ ID NO: 24 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 24.

In some embodiments, provided herein are modified dehalogenase polypeptides comprising a first sequence having at least 70% sequence identity with all or a portion of SEQ ID NO: 18 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 25 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 18. In some embodiments, the first sequence comprises at least 70% sequence similarity with all or a portion of SEQ ID NO: 18 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 18. In some embodiments, the second sequence comprises 100% sequence identity with all or a portion of SEQ ID NO: 25. In some embodiments, the second sequence comprises at least 70% sequence similarity with all or a portion of SEQ ID NO: 25 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 25.

In some embodiments, provided herein are modified dehalogenase polypeptides comprising a first sequence having at least 70% sequence identity with all or a portion of SEQ ID NO: 19 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 26 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 19. In some embodiments, the first sequence comprises at least 70% sequence similarity with all or a portion of SEQ ID NO: 19 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 19. In some embodiments, the second sequence comprises 100% sequence identity with all or a portion of SEQ ID NO: 26. In some embodiments, the second sequence comprises at least 70% sequence similarity with all or a portion of SEQ ID NO: 26 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 26.

In some embodiments, provided herein are modified dehalogenase polypeptides comprising a first sequence having at least 70% sequence identity with all or a portion of SEQ ID NO: 20 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 27 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 20. In some embodiments, the first sequence comprises at least 70% sequence similarity with all or a portion of SEQ ID NO: 20 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 20. In some embodiments, the second sequence comprises 100% sequence identity with all or a portion of SEQ ID NO: 27. In some embodiments, the second sequence comprises at least 70% sequence similarity with all or a portion of SEQ ID NO: 27 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 27.

In some embodiments, provided herein are modified dehalogenase polypeptides comprising a peptide or polypeptide insertion within a loop corresponding to loop 194/195 of SEQ ID NO: 1. In some embodiments, provided herein are modified dehalogenase polypeptides comprising a first sequence having at least 70% sequence identity with all or a portion of one of SEQ ID NOS: 81-85 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of one of SEQ ID NOS: 86-90 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence.

In some embodiments, provided herein are modified dehalogenase polypeptides comprising a first sequence having at least 70% sequence identity with all or a portion of SEQ ID NO: 81 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 86 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 81. In some embodiments, the first sequence comprises at least 70% sequence similarity with all or a portion of SEQ ID NO: 81 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 81. In some embodiments, the second sequence comprises 100% sequence identity with all or a portion of SEQ ID NO: 86. In some embodiments, the second sequence comprises at least 70% sequence similarity with all or a portion of SEQ ID NO: 86 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 86.

In some embodiments, provided herein are modified dehalogenase polypeptides comprising a first sequence having at least 70% sequence identity with all or a portion of SEQ ID NO: 82 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 87 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 82. In some embodiments, the first sequence comprises at least 70% sequence similarity with all or a portion of SEQ ID NO: 82 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 82. In some embodiments, the second sequence comprises 100% sequence identity with all or a portion of SEQ ID NO: 87. In some embodiments, the second sequence comprises at least 70% sequence similarity with all or a portion of SEQ ID NO: 87 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 87.

In some embodiments, provided herein are modified dehalogenase polypeptides comprising a first sequence having at least 70% sequence identity with all or a portion of SEQ ID NO: 83 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 88 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 83. In some embodiments, the first sequence comprises at least 70% sequence similarity with all or a portion of SEQ ID NO: 83 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 83. In some embodiments, the second sequence comprises 100% sequence identity with all or a portion of SEQ ID NO: 88. In some embodiments, the second sequence comprises at least 70% sequence similarity with all or a portion of SEQ ID NO: 88 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 88.

In some embodiments, provided herein are modified dehalogenase polypeptides comprising a first sequence having at least 70% sequence identity with all or a portion of SEQ ID NO: 84 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 89 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 84. In some embodiments, the first sequence comprises at least 70% sequence similarity with all or a portion of SEQ ID NO: 84 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 84. In some embodiments, the second sequence comprises 100% sequence identity with all or a portion of SEQ ID NO: 89. In some embodiments, the second sequence comprises at least 70% sequence similarity with all or a portion of SEQ ID NO: 89 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 89.

In some embodiments, provided herein are modified dehalogenase polypeptides comprising a first sequence having at least 70% sequence identity with all or a portion of SEQ ID NO: 85 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the C-terminus of a peptide or polypeptide insertion sequence and with a second sequence having at least 70% sequence identity with all or a portion of SEQ ID NO: 90 (e.g., >70% sequence identity, >75% sequence identity, >80% sequence identity, >85% sequence identity, >90% sequence identity, >95% sequence identity, >96% sequence identity, >97% sequence identity, >98% sequence identity, >99% sequence identity) fused to the N-terminus of the peptide or polypeptide insertion sequence. In some embodiments, the first sequence comprises 100% sequence identity with all or a portion of SEQ ID NO: 85. In some embodiments, the first sequence comprises at least 70% sequence similarity with all or a portion of SEQ ID NO: 85 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the first sequence comprises 100% sequence similarity with all or a portion of SEQ ID NO: 85. In some embodiments, the second sequence comprises 100% sequence identity with all or a portion of SEQ ID NO: 90. In some embodiments, the second sequence comprises at least 70% sequence similarity with all or a portion of SEQ ID NO: 90 (e.g., >70% sequence similarity, >75% sequence similarity, >80% sequence similarity, >85% sequence similarity, >90% sequence similarity, >95% sequence similarity, >96% sequence similarity, >97% sequence similarity, >98% sequence similarity, >99% sequence similarity). In some embodiments, the second sequence comprises 100% sequence similarity with all or a portion of SEQ ID NO: 90. In some embodiments, provided herein are circular permutations of the modified dehalogenases described herein (e.g., having inserted sequences in the 165 loop and/or 180 loop). In some embodiments, the circularly permuted variant comprises a cp site at a position corresponding to any position between positions 5 and 290 of SEQ ID NO: 1 (e.g., position 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, or 290). In some embodiments, the circularly permuted variant comprises a cp site at a position corresponding to a position between positions 5 and 13 (e.g., 5, 6, 7, 8, 9, 10, 11, 12, 13, or ranges therebetween), 36 and 51 (e.g., 36, 37, 38, 39, 40, 41, 42, 43, 44, 45, 46, 47, 48, 49, 50, 11, or ranges therebetween), 63 and 72 (e.g., 63, 64, 65, 66, 67, 68, 69, 70, 71, 72, or ranges therebetween), 84 and 92 (e.g., 84, 85, 86, 87, 88, 89, 90, 91, 92, or ranges therebetween), 104 and 130 (e.g., 104, 105, 106, 107, 108, 109, 110, 111, 112, 113, 114, 115, 116, 117, 118, 119, 120, 121, 122, 123, 124, 125, 126, 127, 128, 129, 130, or ranges therebetween), 142 and 148 (e.g., 142, 143, 144, 145, 146, 147, 148, and ranges therebetween), 160 and 174 (e.g., 160, 161, 162, 163, 164, 165, 166, 167, 168, 169, 170, 171, 172, 173, 174, or ranges therebetween), 186 and 189 (e.g., 186, 187, 188, 189, or ranges therebetween), 201 and 203 (e.g., 201, 202, 203, or ranges therebetween), 221 and 229 (e.g., 221, 222, 223, 224, 225, 226, 227, 228, 229, or ranges therebetween), or 269 and 290 (e.g., 269, 270, 271, 272, 273, 274, 275, 276, 277, 278, 279, 280, 281, 282, 283, 284, 285, 286, 287, 288, 289, 290, or ranges therebetween), of SEQ ID NO: 1.

In some embodiments, a cp modified dehalogenase comprises a first segment with at least 70% (e.g., 70%, 75%, 80%, 85%, 90%, 95%, 100%) sequence identity to a first portion of one of SEQ ID NOS: 2-5 and a second segment with at least 70% (e.g., 70%, 75%, 80%, 85%, 90%, 95%, 100%) sequence identity to a first portion of one of SEQ ID NOS: 2-5.

In some embodiments, the polypeptides herein retain the capacity of a modified dehalogenase to form a stable bond (e.g., covalent bond) with a haloalkane substrate.

Circularly permuted modified dehalogenase variants (e.g., cpHTs) are described in U.S. Prov. App. No. 63/338,364 and U.S. application Ser. No. 18/311,977, which are incorporated by reference herein in their entireties. In some embodiments, a circularly permuted modified dehalogenase is provided comprising an extended surface loop and/or a loop 165,180, and/or 194/195 insertion. For example, any of the modified dehalogenase sequences provided herein may be provided as circularly permuted versions thereof (e.g., with any suitable cp site described therein). Similarly, any cp modified dehalogenases (e.g., cpHTs) described in U.S. Prov. App. No. 63/338,364 and/or U.S. application Ser. No. 18/311,977 may be provided with an extended surface loop and/or a loop 165, 180, and/or 194/195 insertion.

Split modified dehalogenase variants (e.g., spHTs) are described in U.S. Prov. App. No. 63/338,323 and U.S. application Ser. No. 18/312,117, which are incorporated by reference herein in their entireties. In some embodiments, a split modified dehalogenase is provided comprising an extended surface loop and/or a loop 165, 180, and/or 194/195 insertion. For example, any of the modified dehalogenase sequences provided herein may be provided as split versions thereof (e.g., with any suitable sp site described therein). Similarly, any sp modified dehalogenases (e.g., spHTs) described in U.S. Prov. App. No. 63/338,323 and/or U.S. application Ser. No. 18/312,117 may be provided with an extended surface loop and/or a loop 165, 180, and/or 194/195 insertion.

### II. Inserts

The present invention comprises amino acid sequences (e.g., peptides or polypeptides) inserted into locations with a modified dehalogenase (e.g., SEQ ID NO: 1 or sequence derived therefrom (e.g., >70% sequence identity)).

In some embodiments, the insertion is an extended loop sequence, for example, to enhance/modify interactions between the modified dehalogenase and the substrate (e.g., the functional moiety of the substrate). In some embodiments, the extended loop sequence is of the sequence X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅, wherein each of X₁-X₂₅ are independently selected from any amino acid (e.g., proteinogenic amino acids, natural amino acids, non-natural amino acids, amino acid analogs, etc.) or may be absent. In some embodiments, at least 1 of X₁-X₂₅ are not absent. In some embodiments, X₁-X₂₅ is 1 amino acid in length, 2 amino acids in length, 3 amino acids in length, 4 amino acids in length, 5 amino acids in length, 6 amino acids in length, 7 amino acids in length, 8 amino acids in length, 9 amino acids in length, 10 amino acids in length, 15 amino acids in length, 20 amino acids in length, 25 amino acids in length, or ranges therebetween.

In some embodiments, the insertion is a peptide or polypeptide with a desired functionality. In such embodiments, the peptide or polypeptide may be of any length (e.g., 10 amino acids, 20 amino acids, 30 amino acids, 40 amino acids, 50 amino acids, 75 amino acids, 100 amino acids, 150 amino acids, 200 amino acids, 300 amino acids, 400 amino acids, 500 amino acids, 600 amino acids, 700 amino acids, 800 amino acids, 900 amino acids, 1000 amino acids, or more or ranges therebetween). In some embodiments, because the insertion location is a loop, the substrate binding capacity of the modified dehalogenase is maintained despite the presence of the insertion.

In some embodiments, the insert is a heterologous sequence. In some embodiments, the heterologous sequence interacts (e.g., through contact and/or through resonance/energy transfer) with the functional moiety of the substrate.

Heterologous sequences useful as inserts in modified dehalogenases include, but are not limited to, an enzyme of interest, e.g., luciferase, RNasin or RNase, and/or a channel protein, a receptor, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, a transcription factor, a transporter protein and/or a targeting sequence, e.g., a myristilation sequence, a mitochondrial localization sequence, or a nuclear localization sequence, that directs the modified dehalogenase to a particular location. The heterologous sequence, which is fused within a loop of the modified dehalogenase, may be a fragment of a full protein, e.g., a functional or structural domain of a protein, such as a domain of a kinase, a transcription factor, and the like. A heterologous sequence may be a fragment of a protein that interacts with a second fragment of a protein to form an active complex by protein complementation.

In some embodiments, a heterologous sequence inserted into a loop of a modified dehalogenase interacts with another element to form a complex. For example, FRB or FKBP can be inserted into the 165 of 180 loop and can interact with the other when brought into proximity. Exemplary heterologous sequences include, but are not limited to, sequences such as those in FRB and FKBP, the regulatory subunit of protein kinase (PKa-R) and the catalytic subunit of protein kinase (PKa-C), a src homology region (SH2) and a sequence capable of being phosphorylated, e.g., a tyrosine containing sequence, an isoform of 14-3-3, e.g., 14-3-3t (see Mils et al., 3100), and a sequence capable of being phosphorylated, a protein having a WW region (a sequence in a protein which binds proline rich molecules (see Ilsley et al., 3102; and Einbond et al., 1996) and a heterologous sequence capable of being phosphorylated, e.g., a serine and/or a threonine containing sequence, as well as sequences in dihydrofolate reductase (DHFR) and gyrase B (GyrB).

In some embodiments, a heterologous sequence for insertion into a loop of a modified dehalogenase is selected from the group consisting of an antibody, antibody fragment, protein A, an Ig binding domain of protein A, protein G, an Ig binding domain of protein G, protein A/G, an Ig binding domain of protein A/G, protein L, a Ig binding domain of protein L, protein M, an Ig binding domain of protein M, oligonucleotide probe, peptide nucleic acid, DARPin, anticalin, nanobody, aptamer, affimer, a purified protein, and analyte binding domain(s) of proteins.

As described throughout, any variety of peptides, polypeptides, antibodies, enzymes, reporters, and proteins of interest may be inserted into the 165 and 180 loops of a modified dehalogenase herein. For instance, the invention provides an internal fusion comprising (1) the modified dehalogenase (2) inserted within the 165 of 180 loop, an amino acid sequence for a protein or peptide of interest, e.g., sequences for a marker protein, e.g., a selectable marker protein, an enzyme of interest, e.g., luciferase, RNasin, RNase, and/or GFP, a nucleic acid binding protein, an extracellular matrix protein, a secreted protein, an antibody or a portion thereof such as Fc, a bioluminescence protein, a receptor ligand, a regulatory protein, a serum protein, an immunogenic protein, a fluorescent protein, a protein with reactive cysteines, a receptor protein, e.g., NMDA receptor, a channel protein, e.g., an ion channel protein such as a sodium-, potassium- or a calcium-sensitive channel protein including a HERG channel protein, a membrane protein, a cytosolic protein, a nuclear protein, a structural protein, a phosphoprotein, a kinase, a signaling protein, a metabolic protein, a mitochondrial protein, a receptor associated protein, a fluorescent protein, an enzyme substrate, e.g., a protease substrate, a transcription factor, a protein destabilization sequence, or a transporter protein, e.g., EAAT1-4 glutamate transporter, as well as targeting signals, e.g., a plastid targeting signal, such as a mitochondrial localization sequence, a nuclear localization signal or a myristilation sequence, that directs the fusion to a particular location.

In some embodiments, the heterologous sequence is associated with a membrane or a portion thereof, e.g., targeting proteins such as those for endoplasmic reticulum targeting, cell membrane bound proteins, e.g., an integrin protein or a domain thereof such as the cytoplasmic, transmembrane and/or extracellular stalk domain of an integrin protein, and/or a protein that links the mutant hydrolase to the cell surface, e.g., a glycosylphosphoinositol signal sequence.

Heterologous sequences for insertion into a modified dehalogenase loop may include those having an enzymatic activity. For example, a functional protein sequence may encode a kinase catalytic domain (Hanks and Hunter, 1995), producing a fusion protein that can enzymatically add phosphate moieties to particular amino acids, or may encode a Src Homology 2 (SH2) domain (Sadowski et al., 1986; Mayer and Baltimore, 1993), producing a fusion protein that specifically binds to phosphorylated tyrosines.

In some embodiments, the insert comprises an affinity domain, including peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include HisV5 (HHHHH) (SEQ ID NO:81), HisX6 (HHHHHH) (SEQ ID NO:82), C-myc (EQKLISEEDL) (SEQ ID NO:83), Flag (DYKDDDDK) (SEQ ID NO:84), SteptTag (WSHPQFEK) (SEQ ID NO:85), hemagluttinin, e.g., HA Tag (YPYDVPDYA) (SEQ ID NO:86), GST, thioredoxin, cellulose binding domain, RYIRS (SEQ ID NO: 87), Phe-His-His-Thr (SEQ ID NO: 88), chitin binding domain, S-peptide, T7 peptide, SH2 domain, C-end RNA tag, WEAAAREACCRECCARA (SEQ ID NO:10), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D_{9K}, calbindin D_{28K}, and calretinin, inteins, biotin, streptavidin, MyoD, Id, leucine zipper sequences, maltose binding protein, and SPYTAG peptide or SPYCATCHER protein (e.g.,

(SEQ ID NO: 89)
SYYHHHHHHDYDIPTTENLYFQGAMVTTLSGLSGEQGPSGDMTTE
EDSATHIKFSKRDEDGRELAGATMELRDSSGKTISTWISDGHVKD
FYLYPGKYTFVETAAPDGYEVATPIEFTVNEDGQVTVDGEATEGD
AHTGSSGS,
(SEQ ID NO: 90)
SYYHHHHHHDYDIPTTENLYFQGAMVTTLSGLSGEQGPSGDMTTE
EDSATHIKFSKRDEDGRELAGATMELRDCSGKTISTWISDGHVKD
FYLYPGKYTFVETAAPDGYEVATPIEFTVNEDGQVTVDGEATEGD
AHTGSSGS,
(SEQ ID NO: 91)
GSSHHHHHHSSGLVPRGSRGVPHIVMVDAYKRYKGSGESGKIEEG
KLVIWINGDKGYNGLAEVGKKFEKDTGIKVTVEHPDKLEEKFPQV
AATGDGPDIIFWAHDRFGGYAQSGLLAEITPDKAFQDKLYPFTWD
AVRYNGKLIAYPIAVEALSLIYNKDLLPNPPKTWEEIPALDKELK
AKGKSALMFNLQEPYFTWPLIAADGGYAFKYENGKYDIKDVGVDN
AGAKAGLTFLVDLIKNKHMNADTDYSIAEAAFNKGETAMTINGPW
AWSNIDTSKVNYGVTVLPTFKGQPSKPFVGVLSAGINAASPNKEL
AKEFLENYLLTDEGLEAVNKDKPLGAVALKSYEEELAKDPRIAAT
MENAQKGEIMPNIPQMSAFWYAVRTAVINAASGRQTVDEALKDAQ
TNSSS,

etc.).

In some embodiments, the insert is a fluorescent or luminescent protein. In some embodiments, the insert is a bioluminescent protein. In certain embodiments, the insert is a luciferase. Suitable luciferase enzymes include those selected from the group consisting of: Photinus pyralis or North American firefly luciferase; Luciola cruciata or Japanese firefly or Genji-botaru luciferase; Luciola italic or Italian firefly luciferase; Luciola lateralis or Japanese firefly or Heike luciferase; N. nambi luciferase; Luciola mingrelica or East European firefly luciferase; Photuris pennsylvanica or Pennsylvania firefly luciferase; Pyrophorus plagiophthalamus or Click beetle luciferase; Phrixothrix hirtus or Railroad worm luciferase; Renilla reniformis or wild-type Renilla luciferase; Renilla reniformis Rluc8 mutant Renilla luciferase; Renilla reniformis Green Renilla luciferase; Gaussia princeps wild-type Gaussia luciferase; Gaussia princeps Gaussia-Dura luciferase; Cypridina noctiluca or Cypridina luciferase; Cypridina hilgendorfii or Cypridina or Vargula luciferase; Metridia longa or Metridia luciferase; TurboLuc (Auld et al. Biochemistry 2018, 57, 31, 4700-4706: incorporated by reference in its entirety); Nano-lanterns (Suzuki et al. Nature Communications volume 7, Article number: 13718 (2016); incorporated by reference in its entirety); and Oplophorus luciferase (e.g., Oplophorus gracilirostris (OgLuc luciferase), Oplophorus grimaldii, Oplophorus spinicauda, Oplophorus foliaceus, Oplophorus noraezeelandiae, Oplophorus typus, Oplophorus noraezelandiae or Oplophorus spinous). In some embodiments, a luciferase is selected from those found in Omphalotus olearius, fireflies (e.g., Photinini), Renilla reriformis, Aequoria, mutants thereof, portions thereof, variants thereof, and any other luciferase enzymes suitable for the systems and methods described herein.

In some embodiments, the bioluminescent insert is a modified, enhanced luciferase enzyme from Oplophorus (e.g., NANOLUC enzyme from Promega Corporation, SEQ ID NO: 28 or a sequence with at least 70% identity (e.g., >70%, >80%, >90%, >95%) thereto). Exemplary bioluminescent inserts are described, for example, in U.S. Pat. App. No. 2010/0281552 and U.S. Pat. App. No. 2012/0174242, both of which are herein incorporated by reference in their entireties.

In some embodiments, a modified dehalogenase comprises a loop 165, loop 180, or loop 194/195 insertion of a peptide or polypeptide component of a commercially available NanoLuc®-based technology (e.g., NanoLuc® luciferase, NanoBiT, NanoTrip, NanoBRET, etc.), for example a sequence of one of SEQ ID NOS: 29-31. PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides that find use as heterologous sequences in the fusions herein. In some embodiments, the insert is a circularly permuted version of a NanoLuc®-based component (e.g., NanoLuc® luciferase, NanoBiT, NanoTrip, NanoBRET, etc.). Such polypeptides find use in embodiments herein and can be used in conjunction with the compositions and methods described herein. PCT Appln. No. PCT/US14/26354 and U.S. Pat. No. 9,797,889 (each of which is herein incorporated by reference in their entirety and for all purposes) describe compositions and methods for the assembly of bioluminescent complexes; such complexes, and the peptide and polypeptide components thereof, find use as heterologous sequences in embodiments herein and can be used in conjunction with the compositions and methods described herein. In some embodiments, NanoBiT and other related technologies utilize a peptide component and a polypeptide component that, upon assembly into a complex, exhibit significantly-enhanced (e.g., 2-fold, 5-fold, 10-fold, 10²-fold, 10³-fold, 10⁴-fold, or more) luminescence in the presence of an appropriate substrate (e.g., coelenterazine or a coelenterazine analog) when compared to the peptide component and polypeptide component alone. In some embodiments, the NanoBiT peptides and polypeptides are inserted within a modified dehalogenase herein. U.S. Pat. Pub. 2020/0270586 and Intl. App. No. PCT/US19/36844 (herein incorporated by reference in their entireties and for all purposes) describe multipartite luciferase complexes (e.g., NanoTrip) that find use as heterologous sequences in embodiments herein and can be used in conjunction with the compositions and methods described herein.

In some embodiments, an insert is a circularly permuted version of a protein or polypeptide insert described herein. For example, an insert (e.g., within loop 165, 180, or 194/195) is a circularly permuted NanoLuc-, NanoBiT-, or NanoTrip-based peptide or polypeptide. SEQ ID NOS: 33-80 are exemplary constructs comprising various cpNanoLuc inserted into various positions within loop 165, 180, or 194/195. Other combinations of cpNanoLuc and the insertion sites herein are within the scope herein. In some embodiments, a NanoLuc-based polypeptide with a cp site between any of the following positions is inserted into a loop 165/180 insertion site: 6/7, 12/13, 24/25, 27/28, 49/50, 52/53, 55/56, 64/65, 667/68, 70/71, 79/80, 82/83, 84/85, 86/87, 103/104, 106/107, 120/121, 124/125, 130/131, 145/146, 148/149, or any other sites within a NanoLuc or NanoLuc-based polypeptide. SEQ ID NOS: 91-120 are exemplary constructs comprising various cpLgBiT inserted into various positions within loop 165, 180, or 194/195. Other combinations of cpLgBiT and the insertion sites herein are within the scope herein.

In some embodiments, provided herein are modified dehalogenases comprising insert sequence(s) within loop 165 and/or 180. In some embodiments, the modified dehalogenase comprises insert sequences within both loop 165, loop 180, and loop 194/195. In some embodiments, a modified dehalogenase comprises an insert sequence within one or both of loop 165 and loop 180 and further comprises a C-terminal and/or N-terminal fusion sequence. Any of the inserts described above may also find use as terminal fusions to the extended-loop modified dehalogenases described herein.

### III. Substrates

The modified dehalogenases herein utilize haloalkane substrates. In some embodiments, the substrate is of formula (I): R-linker-A-X, wherein R is a solid surface, one or more functional groups, or absent, wherein the linker is a multiatom straight or branched chain including C, N, S, or O, or a group that comprises one or more rings, e.g., saturated or unsaturated rings, such as one or more aryl rings, heteroaryl rings, or any combination thereof, wherein A-X is a substrate for a dehalogenase, hydrolase, HALOTAG, or a modified dehalogenase system herein (e.g., wherein A is (CH₂)₄₋₂₀ and X is a halide (e.g., Cl or Br)). Suitable substrates are described, for example, in U.S. Pat. Nos. 11,072,812; 11,028,424; 10,618,907; and 10,101,332; incorporated by reference in their entireties. In certain embodiments, X of formula (I) is a methylsulfonamide or trifluoromethylsulfonamide, rather than a halide; such an embodiment results in an exchangeable ligand that reversibly binds to a modified dehalogenase (e.g., HALOTAG). Such ligands are described in, for example, Kompa et al. J. Am. Chem. Soc. 2023, 145, 5, 3075-3083; incorporated by reference in its entirety.

In some embodiments, R is one or more functional groups (such as a fluorophore, biotin, luminophore, or a fluorogenic or luminogenic molecule). Exemplary functional groups for use in the invention include, but are not limited to, an amino acid, protein, e.g., enzyme, antibody or other immunogenic protein, a radionuclide, a nucleic acid molecule, a drug, a lipid, biotin, avidin, streptavidin, a magnetic bead, a solid support, an electron opaque molecule, chromophore, MRI contrast agent, a dye, e.g., a xanthene dye, a calcium sensitive dye, e.g., 1-[2-amino-5-(2,7-dichloro-6-hydroxy-3-oxy-9-xanthenyl)-phenoxy]-2-(2′-am-ino-5′-methylphenoxy)ethane-N,N,N′,N′-tetraacetic acid (Fluo-3), a sodium sensitive dye, e.g., 1,3-benzenedicarboxylic acid, 4,4′-[1,4,10,13-tetraoxa-7,16-diazacyclooctadecane-7,16-diylbis(5-methoxy--6,2-benzofurandiyl)]bis (PBFI), a NO sensitive dye, e.g., 4-amino-5-methylamino-2′,7′-difluorescein, or other fluorophore. In one embodiment, the functional group is an immunogenic molecule, i.e., one which is bound by antibodies specific for that molecule.

In some embodiments, substrates of the invention are permeable to the plasma membranes of cells (i.e., capable of passing from the exterior of a cell (e.g., eukaryotic, prokaryotic) to the cellular interior without chemical, enzymatic, or mechanical disruption of the cell membrane).

In some embodiments, substrates herein comprise a cleavable linker, for example, those described in U.S. Pat. No. 10,618,907; incorporated by reference in its entirety.

In some embodiments, a substrate comprises a fluorescent functional group (R). Suitable fluorescent functional groups include, but are not limited to: stilbazolium derivatives (Marquesa et al. Mechanism-Based Strategy for Optimizing HaloTag Protein Labeling. ChemRxiv. Cambridge: Cambridge Open Engage; 2021; incorporated by reference in its entirety), xanthene derivatives (e.g., fluorescein, rhodamine, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), tetrapyrrole derivatives (e.g., porphin, phtalocyanine, bilirubin, etc.), CF dye (Biotium), BODIPY (Invitrogen), ALEXA FLOUR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), APC, APCXL, RPE, BPE (Phyco-Biotech), autofluorescent proteins (e.g., YFP, RFP, mCherry, mKate), quantum dot nanocrystals, etc.

In some embodiments, a substrate comprises a fluorogenic functional group (R). A fluorogenic functional group is one that produces and enhanced fluorescent signal upon binding of the substrate to a target (e.g., binding of a haloalkane to a modified dehalogenase). By producing significantly increased fluorescence (e.g., 10X, 31X, 50X, 100X, 310X, 500X, 100X, or more) upon target engagement, the problem of background signal is alleviated. Exemplary fluorogenic dyes for use in embodiments herein include the JANELIA FLUOR family of fluorophores, such as:

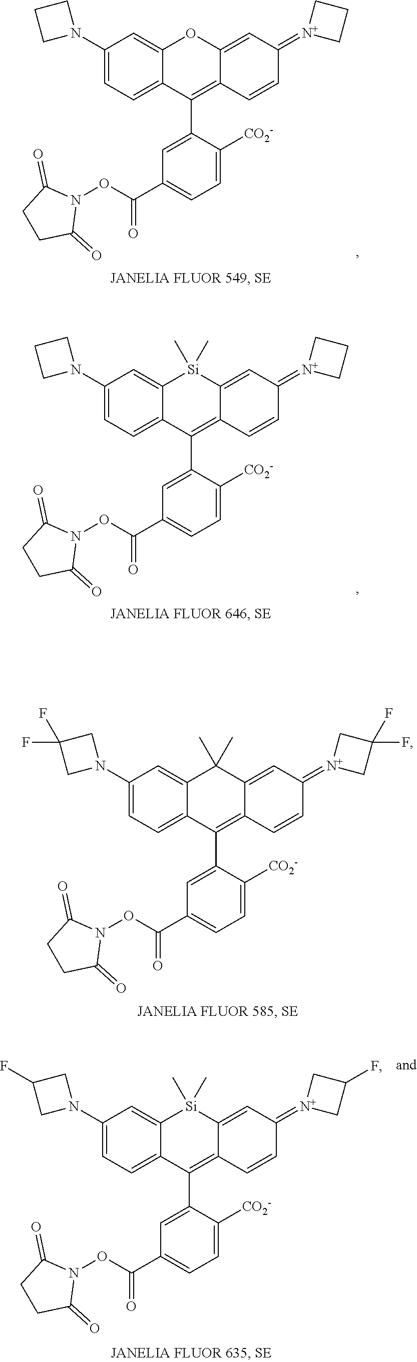

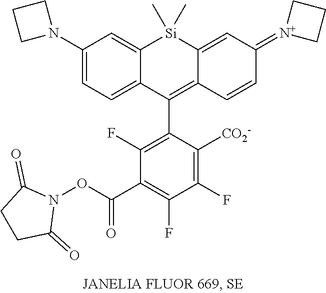

(see, e.g., U.S. Pat. Nos. 9,933,417; 10,018,624; 10,161,932; and 10,495,632; each of which is incorporated by reference in their entireties). In some embodiments, exemplary conjugates of JANELIA FLUOR 549 and JANELIA FLUOR 646 with haloalkane substrates for modified dehalogenase (e.g., HALOTAG) are commercially available (Promega Corp.). The use and design of fluorogenic functional groups, dyes, probes, and substrates is described in, for example Grimm et al. Nat Methods. 3117 October; 14(10):987-994; Wang et al. Nat Chem. 3120 February; 12(2):165-172; incorporated by reference in their entireties.

In some embodiments, ‘dual warhead’ substrates are provided that comprise a haloalkane moiety (e.g., a substrate for a modified dehalogenase (e.g., HALOTAG)) and a dimerization moiety that is a ligand (or capture element) for a second binding protein (capture element). For example, certain embodiments herein utilize a haloalkane linked to a SNAP-tag ligand (Cermakova & Hodges. Molecules 2018, 23(8), 1958; incorporated by reference in its entirety); a haloalkane linked to cTMP (Cermakova & Hodges. Molecules 2018, 23(8), 1958; incorporated by reference in its entirety)); a haloalkane linked to rapamycin-like moiety capable of binding to FKBP or FRB (Chen et al. ACS Chem. Biol. 2021, 16, 12, 2808-2815; incorporated by reference in its entirety); or other haloalkane ‘dual warhead’ ligands capable of binding to a modified dehalogenase (e.g., HALOTAG) and a second capture agent. In such embodiments, a system is provided comprising modified dehalogenase described herein, a dual warhead substrate, and a capture agent capable of binding to the dimerization moiety (e.g., FKBP, FRB, SNAP-tag, eDHFR, etc.). In some embodiments, the insert within the modified dehalogenase and the capture agent are capable of interaction (e.g., structurally or by energy transfer). In some embodiments, by adding another protein binding small molecule moiety onto a haloalkane, the dual warheads trigger close proximity of the inserted heterologous sequence and the capture agent. Such embodiments provide forced proximity of the insert and the capture agent. Any suitable linkers may find use in assembly of dual warhead substrates. The linker may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. For example, in some embodiments, the linker has a formula selected from:

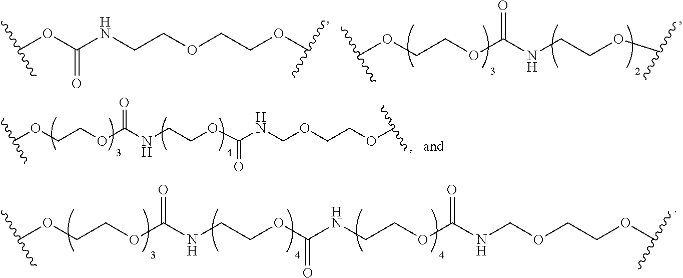

In some embodiments, a dual warhead that finds use in embodiments herein is a haloalkane linked to a ligand capable of engaging an E3 ubiquitin ligase (e.g., thalidomide, Cereblon E3 ubiquitin ligase, von Hippel-Lindau (VHL) E3 ligase or any other E3 ubiquitin ligase), otherwise known as a proteolysis targeting chimera (PROTAC). The haloalkane PROTAC is capable of binding to a modified dehalogenase or modified dehalogenase complex and an E3 ubiquitin ligase; recruitment of the E3 ligase results in ubiquitination and subsequent degradation via the proteasome of the to the modified dehalogenase (complex) and any protein components (e.g., a target protein) fused thereto. In some embodiments, the modified dehalogenase systems herein find use in assays/systems to measure the kinetics of target protein ubiquitination or, in an endpoint format, for applications such as measuring compound dose-response curves. For example, in some embodiments, a sample is provided with a target protein expressed/provided as an insert within the modified dehalogenase; the sample is contacted with a PROTAC of a haloalkane and a ligand capable of engaging an E3 ubiquitin ligase (e.g., thalidomide, Cereblon E3 ubiquitin ligase, von Hippel-Lindau (VHL) E3 ligase or any other E3 ubiquitin ligase); when, the haloalkane is bound by the modified dehalogenase, the ligand in brought into proximity of the target protein, resulting in ubiquitination and directing the fusion target to the proteasome for degradation. In some embodiments, modified dehalogenase systems herein find use in various other targeting chimera (TAC) systems, such as: phosphorylation targeting chimera (PhosTAC; Chen et al. ACS Chem. Biol. 3121, 16, 12, 2808-2815; incorporated by reference in its entirety) systems, deubiquitinase targeting chimera (DUBTAC; Henning et al. Deubiquitinase-Targeting Chimeras for Targeted Protein Stabilization. bioRxiv; 2021. DOI: 10.1101/2021.04.30.441959; incorporated by reference in its entirety) systems, lysosome-targeting chimaera (LyTAC; Banik et al. Nature 584, 291-297 (2020); incorporated by reference in its entirety) systems, autophagy-targeting chimera (AUTAC; Takahashi et al. Mol Cell. 2019 Dec. 5; 76(5):797-810.e10; incorporated by reference in its entirety) systems, autophagy-tethering compound (ATTEC; Fu et al. Cell Research volume 31, pages 965-979 (2021); incorporated by reference in its entirety) systems, and oligo-based TACs. Dual warheads comprising a haloalkane and a ligand for any of the above TAC system may find use in embodiments herein. For example, PhosTACs are similar to the well-described PROTACs in their ability to induce ternary complexes, PhosTACs focus on recruiting a Ser/Thr phosphatase to a phosphosubstrate to mediate its dephosphorylation. PhosTACs extend the use of PROTAC technology beyond protein degradation via ubiquitination to also other protein post-translational modifications. For example, in some embodiments, a target protein is expressed/provided as in insert with a loop of a modified dehalogenase; the sample is contacted with a phosphorylation targeting chimera (PhosTAC) of a haloalkane and a ligand capable of engaging an phosphatase enzyme; upon binding of the haloalkane by the modified dehalogenase the ligand is brought into proximity of the target protein, resulting in phosphorylation of the target protein.

In some embodiments, the modified dehalogenase systems herein find use is other targeting chimera systems in which a dual function ligand comprising a haloalkane and a ligand for a recruitable enzyme is used in combination with modified dehalogenase comprising an inserted target protein to induce the enzymatic activity of the recruitable enzyme to the target protein. Systems and methods comprising any combinations of the above TAC systems/assays are within the scope herein.

In some embodiments, a modified dehalogenase comprises reporter protein inserted within loop 165, loop 180, or loop 194/195 that is capable of emitting energy (e.g., light) at a first wavelength and the functional moiety (R) on the haloalkane substrate comprises a moiety capable of accepting energy at the first wavelength. In some embodiments, the acceptor moiety is a fluorophore. In other embodiments, the acceptor moiety is photocatalyst that is activated by exposure to the emitted energy. In some embodiments, the proximity/geometry between the inserted reporter and acceptor, because of the location of the insert site within the modified dehalogenase, allows for optimized energy transfer.

In some embodiments, the functional moiety (R) on the haloalkane substrate comprises a fluorophore that is capable of absorbing light emitted from a luminophore (upon interaction with a bioluminescent protein or complex (e.g., inserted into a loop of a modified dehalogenase)) and subsequently emitting light. Suitable fluorophores include, but are not limited to, fluorescein and fluorescein dyes (e.g., fluorescein isothiocyanate or FITC, naphthofluorescein, 4′,5′-dichloro-2′,7′-dimethoxy-fluorescein, 6-carboxyfluoresceins (e.g., FAM)), rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine or TMR), coumarin and coumarin dyes (e.g., methoxycoumarin, dialkylaminocoumarin, hydroxycoumarin and aminomethylcoumarin or AMCA), Oregon Green Dyes (e.g., Oregon Green 488, Oregon Green 500, Oregon Green 514), Texas Red, Texas Red-X, SPECTRUM RED™, SPECTRUM GREEN™, cyanine dyes (e.g., CY_3™, CY-5™, CY-3.5™, CY-5.5™), Alexa Fluor dyes (e.g., Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), IRDyes (e.g., IRD40, IRD 700, IRD 800), and the like.

In some embodiments, the functional moiety (R) on the haloalkane substrate comprises a photocatalyst that is capable of absorbing light emitted from a luminophore (upon interaction with a bioluminescent protein or complex (e.g., inserted into a loop of a modified dehalogenase)) and subsequently activating a neighboring activatable label. Any compound or moiety capable of receiving light energy emitted from a bioluminescent protein- or complex-activated luminophore and functionating as a photocatalyst (e.g., transferring that energy to a target molecule (e.g., an activatable molecule)) may find use in embodiments herein. In some embodiments, the excited photocatalyst transfers energy via Forster Resonance Energy Transfer, Dexter Energy Transfer, Single Electron Transfer, Singlet oxygen, or any other suitable mechanism of energy or electron transfer. In some embodiments, the photocatalyst is an iridium-based or ruthenium-based photocatalyst (Bevernaegie et al. ‘A Roadmap Towards Visible Light Mediated Electron Transfer Chemistry with Iridium(III) Complexes.’ ChemPhotoChem 2021, 5, 217; incorporated by reference in its entirety). In some embodiments, the photocatalyst is an organic photoredox catalyst. In some embodiments, the organic photoredox catalyst is selected from a quinone, a pyrylium, an acridinium, a xanthene, and a thiazine. In some embodiments, systems and methods are provided herein comprising a modified dehalogenase comprising a bioluminescent protein or component of a bioluminescent complex inserted into a loop therein, a substrate for a modified dehalogenase comprising a photocatalyst as a functional group, and activatable moiety capable of receiving energy transferred from the photocatalyst.

In addition to the haloalkane substrates describe above and throughout the application (e.g., having the R, linker, A, and X groups described herein), exemplary substrates within the scope herein include:

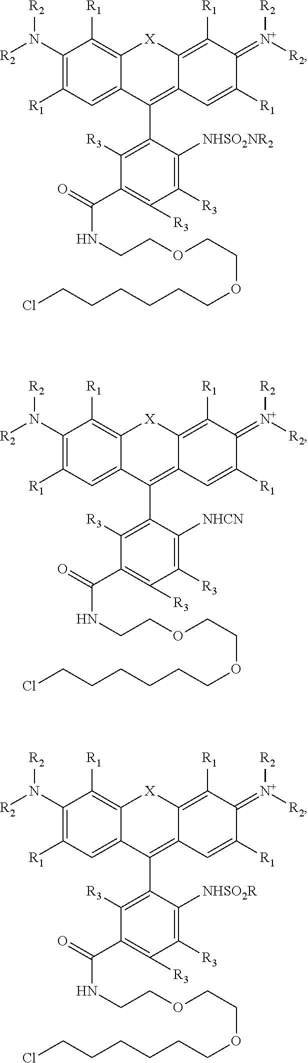

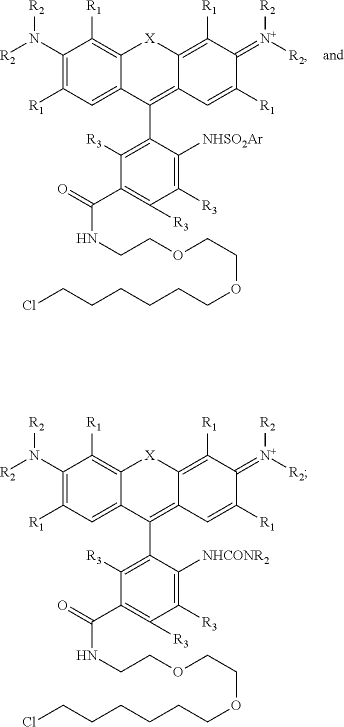

wherein X═O, SiR², or CR²,
wherein R═H, alkyl, or fluoroalkyl;
wherein R² is H, alkyl, cyclized on itself (R² to R²), or cyclized onto R¹;
R¹ is H, alkyl, cyclized onto R², or halogen;
R³ is H, F, or Cl; and
Ar is an aromatic ring (e.g., phenyl), optionally substituted with halogen, OR, NR², CO₂R, CONR², CN, alkyl, or haloalkyl; as described in Wang et al. Nat. Chem. 12, 165-172 (2020); Nat. Chem. 12, 165-172 (2020). and Lardon et al. J. Am. Chem. Soc. 2021, 143, 14592-14600; incorporated by reference in their entioreties.

### IV. Nucleic Acids, Cells, Etc.

In some embodiments, provided herein are isolated nucleic acid molecules (polynucleotides) comprising a nucleic acid sequence encoding the modified dehalogenases (e.g., with internal insertions) described herein. In some embodiments, such polynucleotides contain an open reading frame encoding a modified dehalogenase described herein. In some embodiments, such polynucleotides are within an expression vector or integrated into the genomic material of a cell. In some embodiments, such polynucleotides further comprise regulatory elements such as a promotor. Further provided is an isolated nucleic acid molecule comprising a nucleic acid sequence encoding a fusion protein comprising modified dehalogenase and one or more amino acid residues (e.g., a peptide, a polypeptide) inserted at a location within the 165 or 180 loop(s). In one embodiment, the modified dehalogenase comprises a sequence (e.g., at the N- or C-terminus), for example, for purification, e.g., a glutathione S-transferase (GST) or a polyHis sequence, a sequence intended to alter a property of the remainder of the fusion protein, e.g., a protein destabilization sequence, or a sequence which has a property which is distinguishable. In one embodiment, the isolated nucleic acid molecule comprises a nucleic acid sequence, which is optimized for expression in at least one selected host. Optimized sequences include sequences, which are codon optimized, i.e., codons that are employed more frequently in one organism relative to another organism, e.g., a distantly related organism, as well as modifications to add or modify Kozak sequences and/or introns, and/or to remove undesirable sequences, for instance, potential transcription factor binding sites. In one embodiment, the polynucleotide includes a nucleic acid sequence encoding a modified dehalogenase, which nucleic acid sequence is optimized for expression in a selected host cell. In one embodiment, the optimized polynucleotide no longer hybridizes to the corresponding non-optimized sequence, e.g., does not hybridize to the non-optimized sequence under medium or high stringency conditions. In another embodiment, the polynucleotide has less than 90%, e.g., less than 80%, nucleic acid sequence identity to the corresponding non-optimized sequence and optionally encodes a polypeptide having at least 80%, e.g., at least 85%, 90% or more, amino acid sequence identity with the polypeptide encoded by the non-optimized sequence.

Constructs, e.g., expression cassettes, and vectors comprising the isolated nucleic acid molecule, as well as host cells having one or more of the constructs, and kits comprising the isolated nucleic acid molecule, one or more constructs or vectors are also provided. Host cells include prokaryotic cells or eukaryotic cells such as a plant or vertebrate cells, e.g., mammalian cells, including but not limited to a human, non-human primate, canine, feline, bovine, equine, ovine or rodent (e.g., rabbit, rat, ferret, or mouse) cell. In some embodiments, the expression cassette comprises a promoter, e.g., a constitutive or regulatable promoter, operably linked to the nucleic acid molecule. In some embodiments, the expression cassette contains an inducible promoter. In certain embodiments, the invention includes a vector comprising a nucleic acid sequence encoding a fusion protein comprising a fragment of a dehalogenase. In some embodiments, optimized nucleic acid sequences, e.g., human codon optimized sequences, encoding at least a fragment of the hydrolase, and preferably the fusion protein comprising the fragment of a hydrolase, are employed in the nucleic acid molecules of the invention. The optimization of nucleic acid sequences is known to the art, see, for example WO 02/16944; incorporated by reference in its entirety.

Also provided are cells comprising the modified dehalogenases (e.g., with loop 165, loop 180, and/or loop 194/195 insertions), polynucleotides, expression vectors, etc. herein. In some embodiments, a component described herein is expressed within a cell. In some embodiments, a component herein is introduced to a cell, e.g., via transfection, electroporation, infection, cell fusion, or any other means.

### V. Systems and Methods

In some embodiments, provided herein are systems and methods that comprise or utilize a modified dehalogenase comprising an internal insertion within the 165 or 180 loop, or a sequence corresponding thereto. In some embodiments, systems and methods further comprise additional components, such as substrates, binding proteins (e.g., capable of binding to the insert), luminophores, complementary comparisons (e.g., to a bioluminescent complex with an insert of the modified dehalogenase), and other agents/reagents described herein. In some embodiments, methods herein comprise steps of contacting a modified dehalogenase described herein with a substrate and/or additional reagents (e.g., a luminophore), detecting fluorescence/luminescence, isolating/purifying a component, etc.

Particular embodiments herein find use in energy transfer systems and applications. In some embodiments, the modified dehalogenases herein, comprising an internal insertion of a bioluminescent protein or component of a bioluminescent complex within the 165,180, or 194/195 loop, are useful for energy transfer to an appropriate acceptor (e.g., an energy acceptor as the functional moiety (R) on a HALOTAG substrate. In some embodiments, the energy acceptor is a fluorophore or photocatalyst. In some embodiments, the energy acceptor further transfers energy to a second acceptor. For example, in some embodiments, the first acceptor is a first fluorophore with an excitation spectra that overlaps the emission spectra of the bioluminescent protein or bioluminescent complex, and the second acceptor is a second fluorophore with an excitation spectra that overlaps the emission spectra of the first fluorophore. In some embodiments, upon contacting the bioluminescent protein or bioluminescent complex with an appropriate luminophore, energy is transferred from the luminophore to the first fluorophore by BRET and from the first fluorophore to the second fluorophore by FRET. In other embodiments, the first acceptor is a photocatalyst with an excitation spectra that overlaps the emission spectra of the bioluminescent protein or bioluminescent complex, and the second acceptor is a activatable target that is activated by the photocatalyst.

### EXPERIMENTAL

Although loop regions in proteins are often more tolerant to sequence insertion, it was not immediately apparent that loop regions in the commercially-available modified dehalogenase HALOTAG would accommodate alteration without disrupting protein folding or function. A known prior modification (Hiblot, J., et al. (2017) Angew Chem Int Ed Engl 56(46): 14556-14560; incorporated by reference in its entirety) revealed that insertion of the commercially-available NANOLUC luciferase in loop-165 resulted in a functional HALOTAG, but not in loop-180. However, the activity of the resulting construct was heavily dependent on the specific configuration in that example, being sensitive to the specific residue of insertion, linkers, and whether NANOLUC was circularly permuted or not.

Sequences ofpeptides and polypeptides used in, for example, Examples 1-4 are provided in Table 1 (TABLE_1_Loop_HTs.txt filed herewith and incorporated by reference in its entirety) and Table 2.

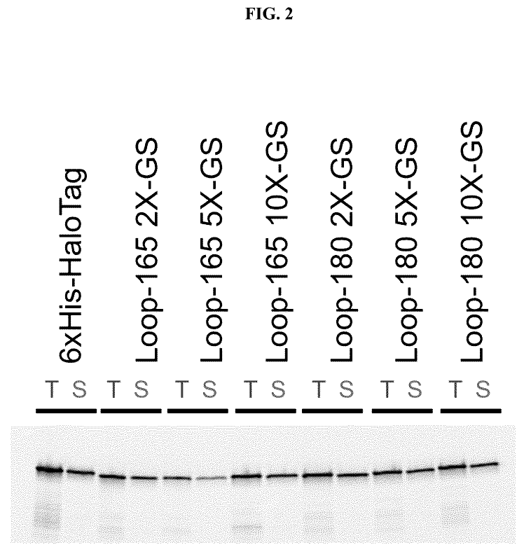

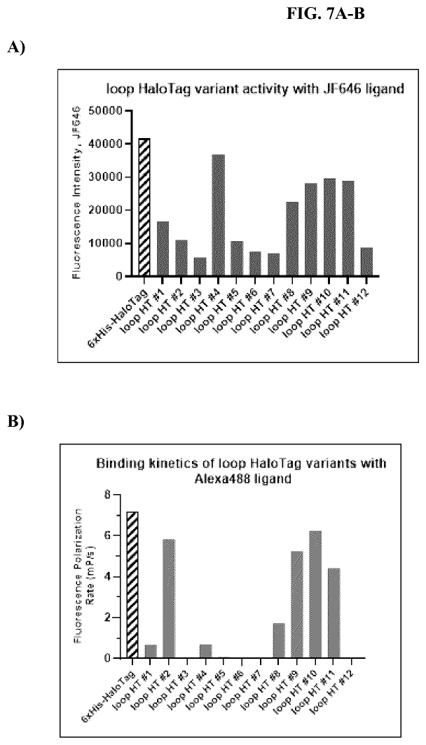

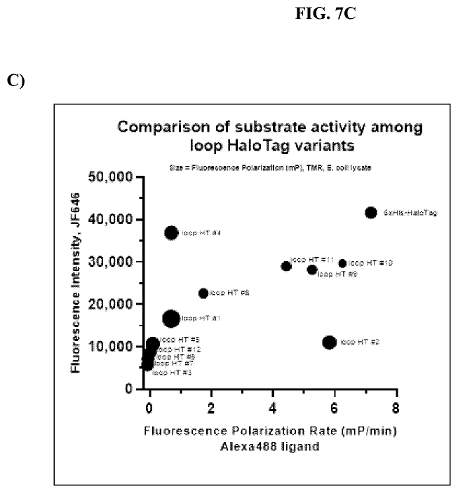

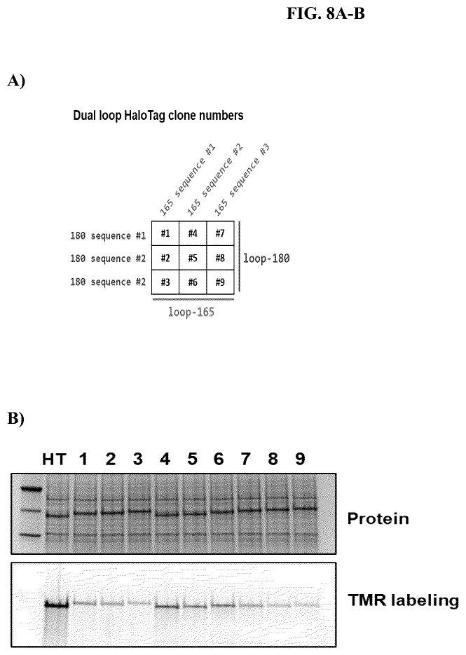

TABLE 2

| | | | Exam- | SEQ |
|---|---|---|---|---|
| Purpose | Name | FIG. | ple | ID NO |
| Linker tolerance | HT_165_2xlinker | FIG. 2, 3 | Ex. 1 | 441 |
| Linker tolerance | HT_165_5xlinker | FIG. 2, 3 | Ex. 1 | 442 |
| Linker tolerance | HT_165_10xlinker | FIG. 2, 3 | Ex. 1 | 443 |
| Linker tolerance | HT_180_2xlinker | FIG. 2, 3 | Ex. 1 | 444 |
| Linker tolerance | HT_180_5xlinker | FIG. 2, 3 | Ex. 1 | 445 |
| Linker tolerance | HT_180_10xlinker | FIG. 2, 3 | Ex. 1 | 446 |
| Linker position | HT_165_v2 | FIG. 4 | Ex. 2 | 447 |
| Linker position | HT_165_v3 | FIG. 4 | Ex. 2 | 448 |
| Linker position | HT_165_v4 | FIG. 4 | Ex. 2 | 449 |
| Linker position | HT_165_v5 | FIG. 4 | Ex. 2 | 450 |
| Linker position | HT_165_v6 | FIG. 4 | Ex. 2 | 451 |
| Linker position | HT_165_v7 | FIG. 4 | Ex. 2 | 452 |
| Linker position | HT_180_v2 | FIG. 4 | Ex. 2 | 453 |
| Linker position | HT_180_v3 | FIG. 4 | Ex. 2 | 454 |
| Linker position | HT_180_v4 | FIG. 4 | Ex. 2 | 455 |
| Linker position | HT_180_v5 | FIG. 4 | Ex. 2 | 456 |
| Linker position | HT_180_v6 | FIG. 4 | Ex. 2 | 457 |
| Single loop screen | loop HT #1 | FIG. 7 | Ex. 3 | 458 |
| Single loop screen | loop HT #2 | FIG. 7 | Ex. 3 | 459 |
| Single loop screen | loop HT #3 | FIG. 7 | Ex. 3 | 460 |
| Single loop screen | loop HT #4 | FIG. 7 | Ex. 3 | 461 |
| Single loop screen | loop HT #5 | FIG. 7 | Ex. 3 | 462 |
| Single loop screen | loop HT #6 | FIG. 7 | Ex. 3 | 463 |
| Single loop screen | loop HT #7 | FIG. 7 | Ex. 3 | 464 |
| Single loop screen | loop HT #8 | FIG. 7 | Ex. 3 | 465 |
| Single loop screen | loop HT #9 | FIG. 7 | Ex. 3 | 466 |
| Single loop screen | loop HT #10 | FIG. 7 | Ex. 3 | 467 |
| Single loop screen | loop HT #11 | FIG. 7 | Ex. 3 | 468 |
| Single loop screen | loop HT #12 | FIG. 7 | Ex. 3 | 469 |
| Dual loop | Dual loop HT #1 | FIG. 8 | Ex. 4 | 470 |
| insertion | | | | |
| Dual loop | Dual loop HT #2 | FIG. 8 | Ex. 4 | 471 |
| insertion | | | | |
| Dual loop | Dual loop HT #3 | FIG. 8 | Ex. 4 | 472 |
| insertion | | | | |
| Dual loop | Dual loop HT #4 | FIG. 8 | Ex. 4 | 473 |
| insertion | | | | |
| Dual loop | Dual loop HT #5 | FIG. 8 | Ex. 4 | 474 |
| insertion | | | | |
| Dual loop | Dual loop HT #6 | FIG. 8 | Ex. 4 | 475 |
| insertion | | | | |
| Dual loop | Dual loop HT #7 | FIG. 8 | Ex. 4 | 476 |
| insertion | | | | |
| Dual loop | Dual loop HT #8 | FIG. 8 | Ex. 4 | 477 |
| insertion | | | | |
| Dual loop | Dual loop HT #9 | FIG. 8 | Ex. 4 | 478 |
| insertion | | | | |

### Example 1

A circular permutation (CP) screen of HALOTAG was conducted during development of embodiments herein to systematically test the effect of circular permutation at all 297 individual positions. Data from the screen showed that HALOTAG could be circularly permuted and new N- and C-termini could be introduced into the loops 165- and 180-loops, retaining HALOTAG function and only minimally impacting protein stability. The screening data showed a clear optimum position for circular permutation in these loops, specifically after residues 165 and 180 in each loop, respectively. Moving the CP site only 2 residues N- or C-terminal of these sites showed losses in activity or stability in HALOTAG, indicating the identification of optimal positions.

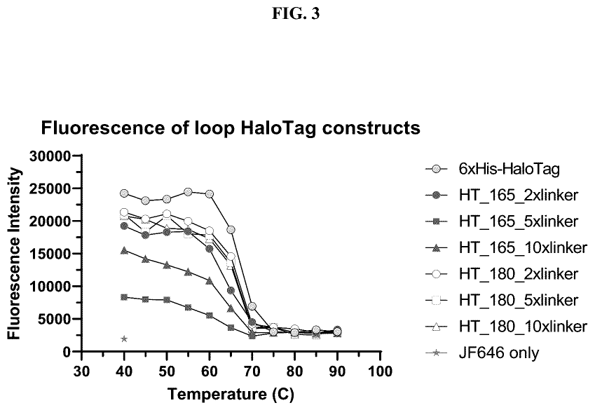

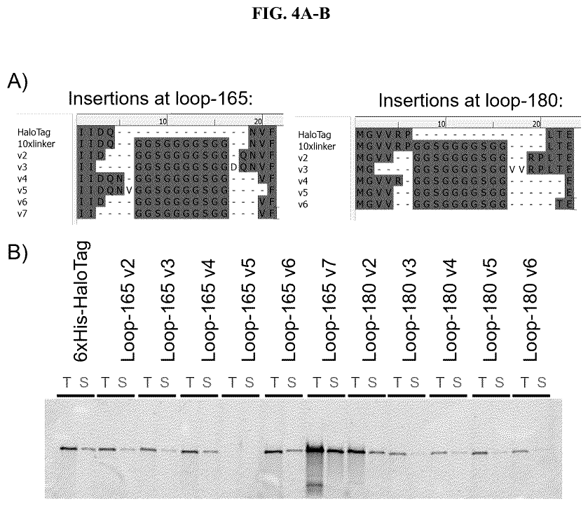

With the CP screening data as a guide, the tolerances of sequence insertion were tested at specific sites within he HALTOTAG sequence by introducing extensions of 2, 5, or 10 residues comprised of Glycine-Serine in each loop separately. Both loop-165 and loop-180 tolerated these extensions, retaining labeling activity with TMR and JF646 ligands (FIG. 2, FIG. 3). The longer Gly-Ser extensions of 5-10 residues in loop-165 resulted in a decrease in protein stability and lower fluorescence activation of JF646 ligand while retaining activity with TMR ligand. This was the first evidence that insertions in these loops specifically modulate activation of fluorogenic dyes.

### Example 2

Experiments were conducted during development of embodiments herein to test for optimal positioning and composition of the loop insertion by sliding the insertion site of a 10x-Gly-Ser extension into loop-165 or loop-180 (FIG. 4A). There was a clear preference for the specific site of extension in terms of retaining expression and enzymatic activity both in E. coli lysates and with purified protein. For loop-165, even though the HaloTag version 7 (“v7”) construct expressed the best, purification of constructs confirmed that the HaloTag version 6 (“v6”) construct was optimal in which the loop is inserted immediately after D164 while simultaneously deleting residues Q165 and N166. For loop-180, the optimal site was the HaloTag version v2 (“v2”) construct where the loop is inserted immediately after V178 (FIG. 4B). Results indicated that insertion of sequences into different locations within loop 165 and loop 180 produces variants with differing performance and characteristics, with some insertion points resulting in low expression or activity proteins.

### Example 3

After establishing an optimal site/configuration of extended loop insertion at loop-165 and loop-180, experiments were conducted using libraries with randomized amino acids in the loop insertion site to determine the tolerance of extended loop modified dehalogenases to varied amino acid loop compositions and suitability for screening/selections that would enable the discovery of optimal sequences for specific applications (Table 1). Eight different library designs were tested:
1. 165-7X/180=7 randomized amino acids inserted at loop-165
2. 165-11X/180=11 randomized amino acids inserted at loop-165
3. 165-15X/180=15 randomized amino acids inserted at loop-165
4. 165/180-7X=7 randomized amino acids inserted at loop-180
5. 165/180-11X=11 randomized amino acids inserted at loop-180
6. 165/180-15X=15 randomized amino acids inserted at loop-180
7. 165-7X/180-7X=7 randomized amino acids inserted at both loop-165 and loop-180
8. 165-15X/180-15X=15 randomized amino acids inserted at both loop-165 and loop-180

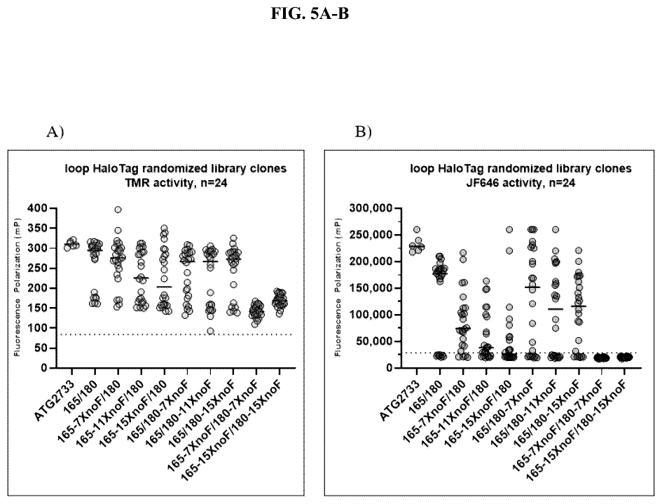

Results are depicted in FIG. 5. The trend in library designs shows that at loop-165, longer loops as a group tend to show decreased total enzymatic activity and activation of JF646, although there are clones that are similar to HALOTAG, even with 15× randomized loop inserted (FIGS. 5A and 5B) demonstrating the impact of specific sequences at these sites. For loop-180, many more clones showed activities similar to HALOTAG, with approximately half showing reduced or no enzymatic activity with the TMR and JF6464 ligands tested. Constructs with 7 or 15 randomized amino acids inserted into both loop-165 and loop-180 had activity eliminated.

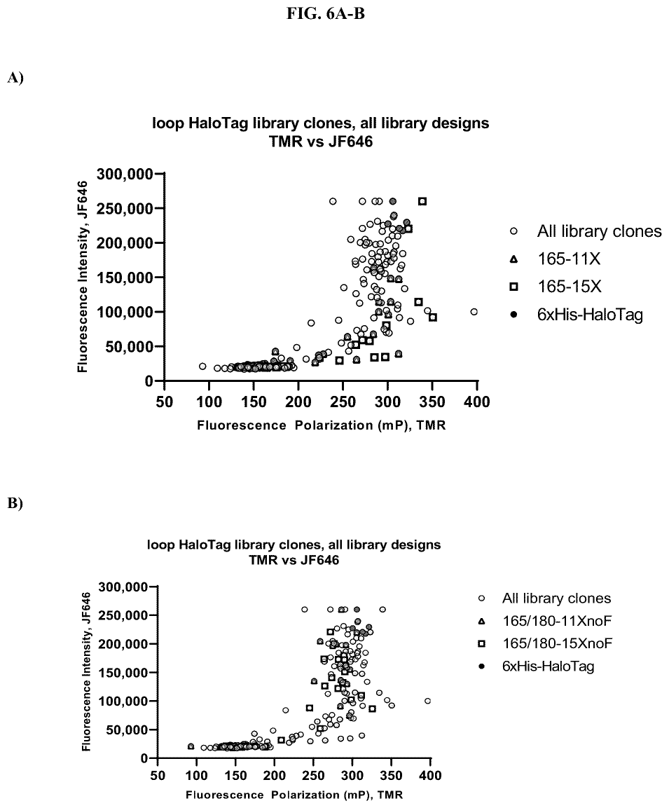

The libraries with randomized loops showed that diverse sequences can be inserted into loop-165 and loop-180 while retaining activity, indicating a large amount of flexibility in the potential to engineer or screen for those that improve function toward specific applications. When comparing the activity of individual loop variants among their activities with TMR versus JF646 ligand, variants were found that show robust binding to TMR ligand and a range of activation levels of the fluorogenic JF646 ligand, from complete loss of activation up to high levels similar to unmodified HALOTAG (FIGS. 6A and 6B). This set of variants confirmed that sequence insertion at loop-165 or loop-180 can control fluorogenic activation of dyes without impacting the enzymatic function of HALOTAG, providing the ability to fine-tune the amount of fluorescence activation of the JF646 ligand using only changes to the residues in the extended loop sequences. The experiments indicate that other activatable chemistries are also tunable on the surface of HALOTAG, where changes to the proximal loop sequences modulate interactions that optimize activation.

More detailed characterization of several loop HALOTAG variants isolated through initial screening showed significant differences among variants in their substrate specificity and kinetics. For example, comparison of various loop HALOTAG clone activities for JF646 vs Alexa488 ligand in FIGS. 7A and 7B shows that loop HALOTAG #2 has low JF646 activity, but high Alexa488 binding, whereas loop HALOTAG #4 has high JF646 binding, but low Alexa488 binding. This demonstrates that changes to sequences only in the loops is sufficient for altering the substrate specificity and binding rates of loop HALOTAG variants.

### Example 4

Experiments were conducted during development of embodiments herein to engineer extended loop insertions within both loops 165 and 180 simultaneously, and it was observed in randomized libraries that dual insertion eliminated HALOTAG activity with the small sample size of randomized sequences tested. However, using specific sequences that retain full stability and function at each insertion site individually, combinations of sequences were tested to determine if their stabilizing effects are synergistic (FIG. 8A). It was observed that sequences that provide high activity loop HALOTAG variants at either 165 or 180 can be combined together to provide active, dual loop HALOTAG clones (FIG. 8B).

### Example 5

Experiments conducted during development of embodiments herein indicate several possible mechanisms behind the observed activation effects for the loop HALOTAG variants. In a direct interaction model, the extended loop sequences have direct contacts with the surface-exposed dye portion of the ligand, and those interactions modulate fluorescence activation. In an indirect interaction model, the extended loop insertion impacts other protein:dye interactions or ligand binding, such as changing positioning of the flanking Helix 8 that has close contacts with the dye in the crystal structure and modulating its level of activation or impacting contacts with the chloroalkane moiety during binding. In some embodiments, a combined direct/indirect model produces the effects.

### Example 6

After establishing the tolerance of loop-165 and loop-180 for small 7-15 amino acids insertions, experiments were conducted to explore the feasibility for significantly larger insertions. To this end, into loop-165-V6 and loop-180-V2 different bioluminescent reporters were inserted, including:
1. NANOLUC, circularly permuted at position 67/68 (cpNLuc)
2. Thermostable NANOLUC (i.e., NanoLuc incorporating all the LgBiT and HiBiT mutations), circularly permuted at position 67/68 (cptsNLuc)
3. Thermostable NanoLuc (i.e., NanoLuc incorporating all the LgBiT and HiBiT mutations)+mutation F164C, circularly permuted at position 67/68 (cptsNLuc(F164C)

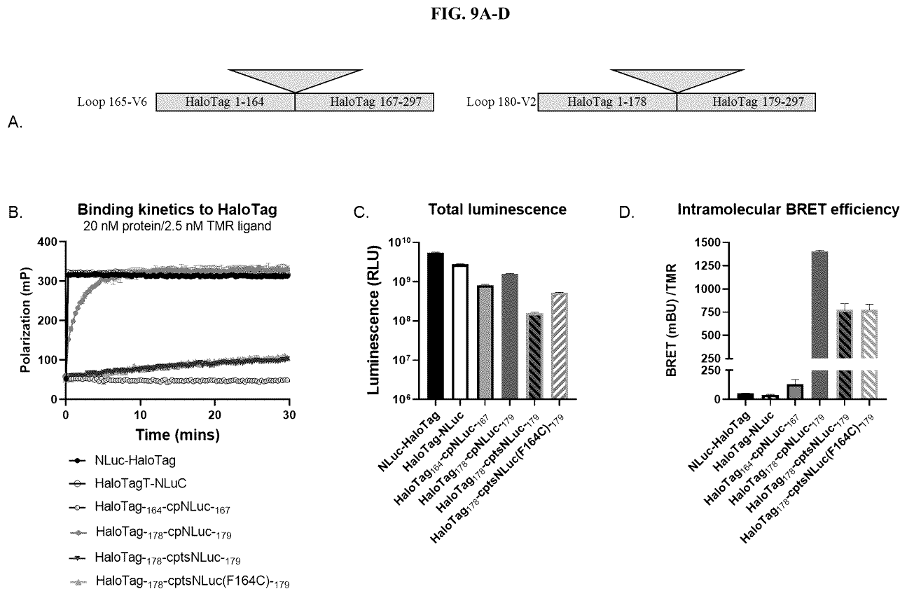

Comparing the resulting HALOTAG-NANOLUC chimeras to terminal HALOTAG-NANOLUC fusions (FIG. 9), it was found that the chimeras exhibited slower binding kinetics to HALOTAG TMR ligand (FIG. 9B) and were dimmer (FIG. 9C) for NANOLUC luminescence, but at the same time delivered greater BRET efficiencies (FIG. 9D), presumably through closer proximity and/or conformations more favorable for energy transfer to a bound TMR ligand. While both loops were tolerant to the larger insertions, the resulting chimeras had different activity profiles. Consistent with previous results, insertion of cpNLuc into loop 180-V2 resulted with significantly smaller impact on binding kinetics to HALOTAG ligands compared to the same insertion into loop 165-V6 (FIG. 9B). The insertion into loop 180-V2 also provided greater increase in BRET efficiency indicating that the chimera was able to adopt a conformation more favorable for energy transfer to a bound TMR ligand (FIG. 9D). The differential HALOTAG ligand binding kinetics and BRET efficiencies for insertions into the two loops could be further leveraged toward orthogonality.

Comparing chimeras comprising insertions of cpNLuc, cptsNLuc, and cptsNLuc(F164C) into loop 180-V2, it was found that increased thermostability of the inserted polypeptide (i.e., cptsNLuc) was correlated with significantly slower binding kinetics to HaloTag ligands (FIG. 9B) and to a lesser extent lower BRET efficiency (FIG. 9D), indicating that engineering greater flexibility/lower stability into the insertion may facilitate adoption of conformations favorable for both HALOTAG activity and energy transfer.

### Example 7

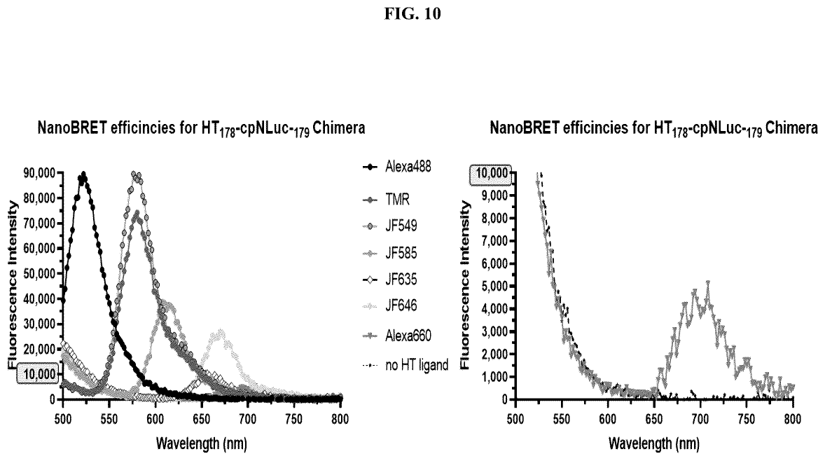

Experiments were conducted during development of embodiments herein to further explore the capacity of chimera comprising insertion of cpNLuc into loop 180 to deliver increased intramolecular BRET efficiency not only to a bound TMR ligand but also to other bound fluorophores exhibiting a wide range of overlaps between their excitation spectrum and the emission of the bioluminescent reporter. Thus far, the chimera comprising insertion of cpNLuc into loop180-V2 showed not only significant increase in BRET efficiency to a bound TMR but also to other fluorophores including fluorogenic fluorophores (i.e., JF635 and JF646) and far-red fluorophores (i.e., Alexa 660) having minimal overlap between their excitation spectrum and the bioluminescent reporter emission (FIG. 10).

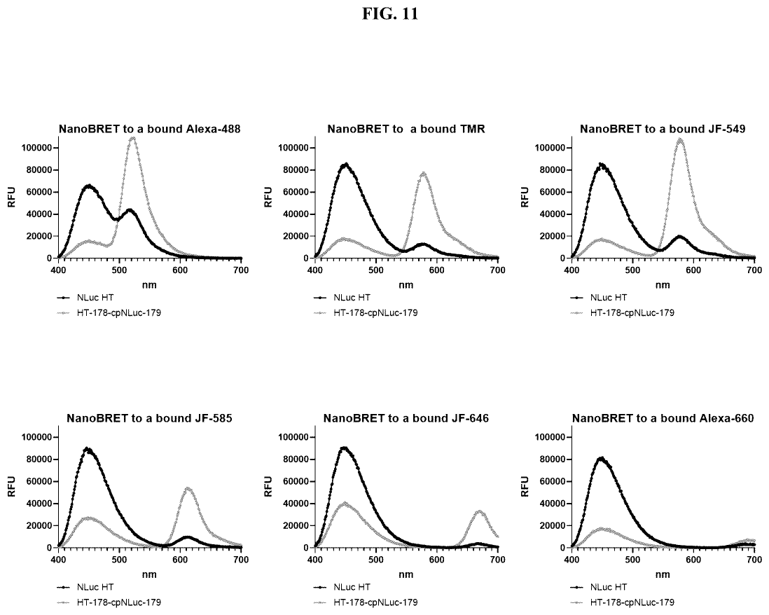

Experiments were conducted during development of embodiments herein to further compare purified NanoLuc-HaloTag fusion and chimera comprising insertion of cpNLuc into loop 180 for their capacity to deliver intramolecular BRET efficiency to bound fluorophores exhibiting a wide range of overlaps between their excitation spectrum and the emission of the bioluminescence reporter. Emission of bioluminescence energy donors and acceptors demonstrated that while the donor emission intensity (i.e., emission at 460 nm) for the chimera was significantly lower compared to NLuc-HaloTag emission intensity for all six fluorophores including fluorogenic fluorophores (i.e., JF635 and JF646) and far-red fluorophores (i.e. Alexa 660) were significantly higher demonstrating the benefit offered by the chimera presumably through closer proximity and/or conformations more favorable for energy transfer to a bound fluorophore (FIG. 11).

### Example 8

Experiments were conducted during development of embodiments herein to further explore insertions of bioluminescent complementation reporters into loop 180 including:
1. A polypeptide component of a NANOLUC-based complementation system (LgBiT)
2. A polypeptide component of a NANOLUC-based complementation system, circularly permuted at position 67/68 (i.e., cpLgBiT).
3. A polypeptide component of a NANOLUC-based complementation system, incorporating four LgTrip mutations (E4D, Q42M, M106K, T144D) (LgBiT+4), circularly permuted at position 67/68 (i.e., cpLgBiT+4).

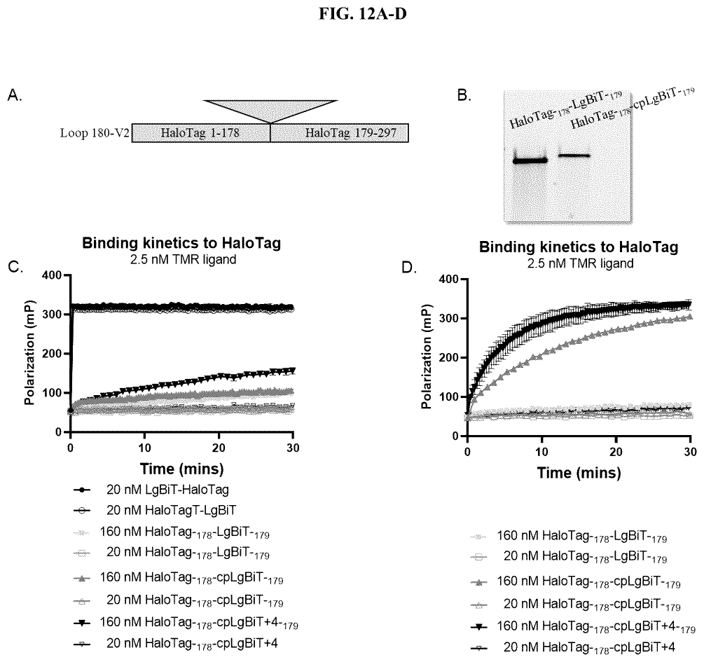

While insertion of LgBiT or cpLgBiT into loop 180-V2 drastically decreased the binding kinetics to HaloTag TMR ligand (FIG. 12C), the resulting chimeras were very different in their binding kinetics upon complementation with 10-fold excess VS-HiBiT (a peptide component of a NANOLUC-based complementation system) (FIG. 12). Overall, the chimeras comprising insertion of cpLgBiT or cpLgBiT+4 exhibited significantly faster binding of a TMR ligand upon complementation with 10-fold molar excess VS-HiBiT (FIG. 12D), suggesting complementation facilitated adaption of a conformation favorable for HaloTag activity. Such dependence of HaloTag binding in complementation could be further leveraged as an HaloTag activity switch. On the contrary, the chimera comprising insertion of LgBiT could be labeled to completion following overnight incubation with 5-fold molar excess of TMR ligand (FIG. 12B), but binding was not accelerated by pre-complementation with VS-HiBiT.

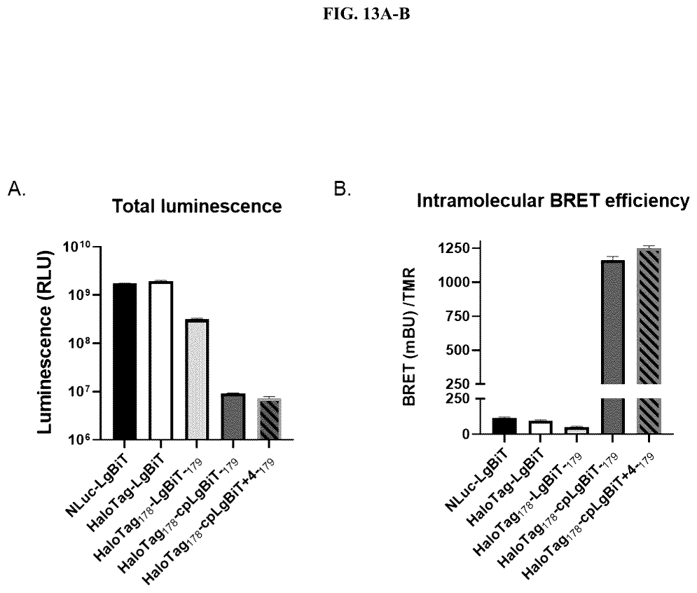

In addition, upon complementation with 10-fold excess VS-HiBiT, the three chimeras were very different in their brightness and efficiencies of intramolecular BRET to a bound TMR ligand (FIG. 13). Although the chimeras comprising insertion of cpLgBiT or cpLgBIT+4 were 2-logs dimmer (FIG. 13A), they delivered 20-fold greater BRET efficiency (FIG. 13B) further suggesting that engineering greater flexibility/lower stability into the insertion may facilitate adoption of conformations favorable not only for complementation and HaloTag activity but also BRET.

### Example 9

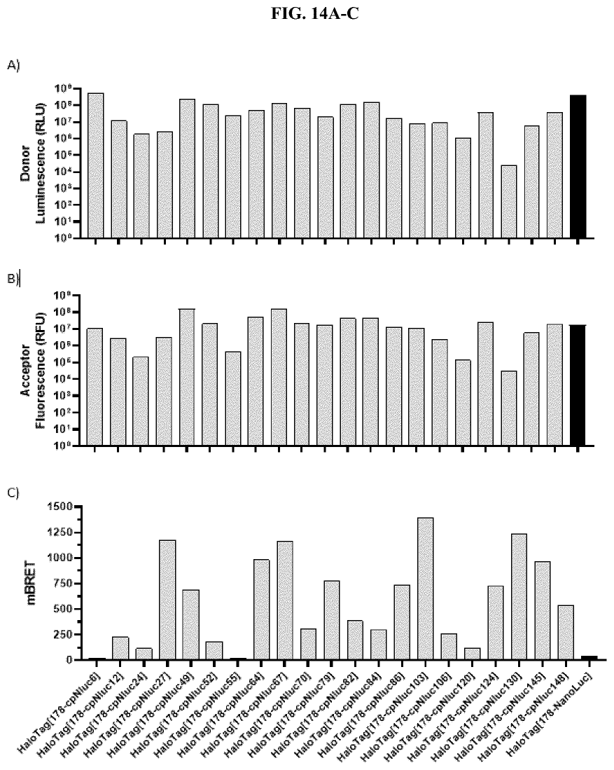

Having determined that insertion of NanoLuc into loop-180 of HaloTag resulted in both enzymes retaining function and an improvement in energy transfer through BRET, experiments were conducted to test a panel of constructs comprising different circularly permuted variants of NanoLuc inserted into HaloTag at loop-180 (FIG. 14). All but two of the tested cpNanoLuc insertion designs significantly improved the BRET ratio over the control of the non-permuted NanoLuc insertion. Examples of the success of this strategy are highlighted by insertion of cpNanoLuc49 and cpNanoLuc67, where both NanoLuc luminescence and energy transfer through BRET are increased significantly over the NanoLuc insertion control. More broadly, the overall number of successful circular permutations indicates that many configurations of protein insertions into the HaloTag loops are possible and make efficient sites for positioning fusion partners in close proximity to the bound CA ligand of HaloTag.

### Example 10

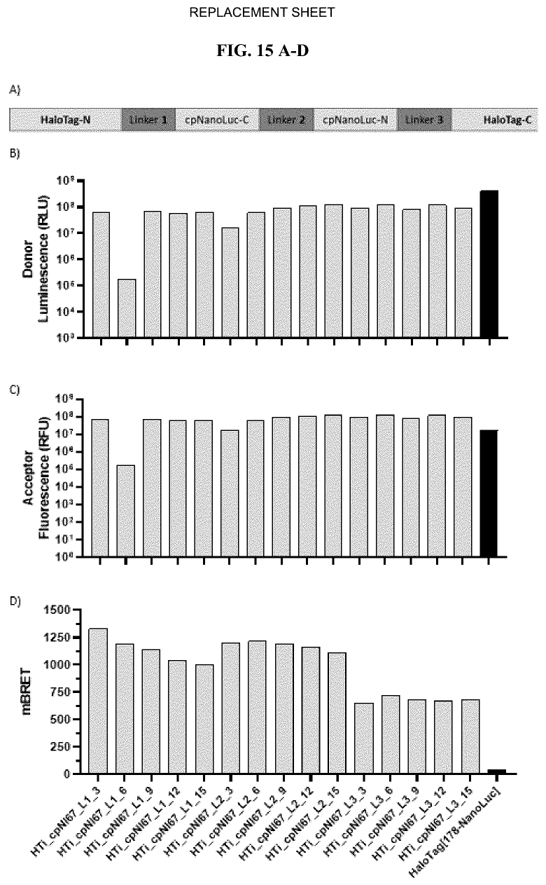

Given that positioning and geometric constraints are critical to the folding, activity, and potential efficiency for energy transfer among component enzymes in a fusion or chimera, experiments were conducted during development of embodiments herein to test a panel of constructs based on the HaloTag-cpNanoLuc67 construct with an insertion at loop-180 that had different sized flexible Glycine-Serine linkers flanking different components (FIG. 15). Loop-180 continued to tolerate further modification beyond insertion of cpNanoLuc67 by insertion of additional linkers of lengths ranging from 3-15 Gly-Ser residues. All of the linker variants were functional for HaloTag and NanoLuc activity and exhibited a range of BRET ratios. Insertion of flexible linkers in particular to either the sequence immediately N-terminal of the cpNanoLuc67 insertion or within the cpNanoLuc67 itself retained >80% of the BRET ratio relative to the construct without linkers. This indicates loop-180 of HaloTag can tolerate both insertion of linkers and polypeptides simultaneously, enabling flexibility in the nature and composition of elements that can positioned close to the bound CA-ligand.

### Example 11

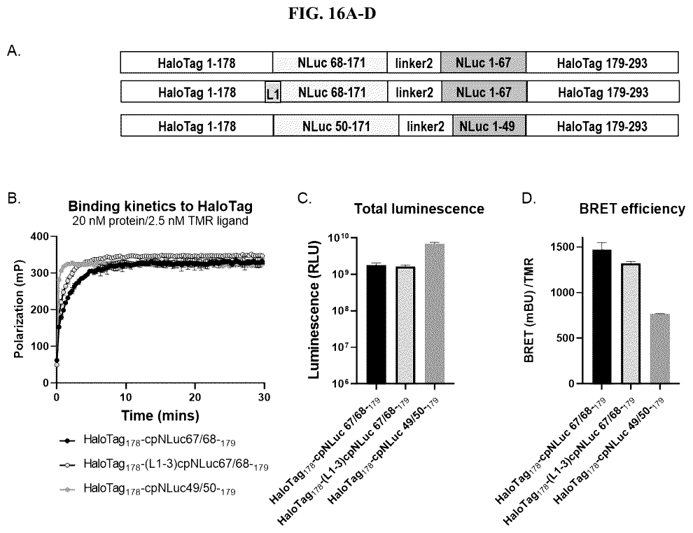

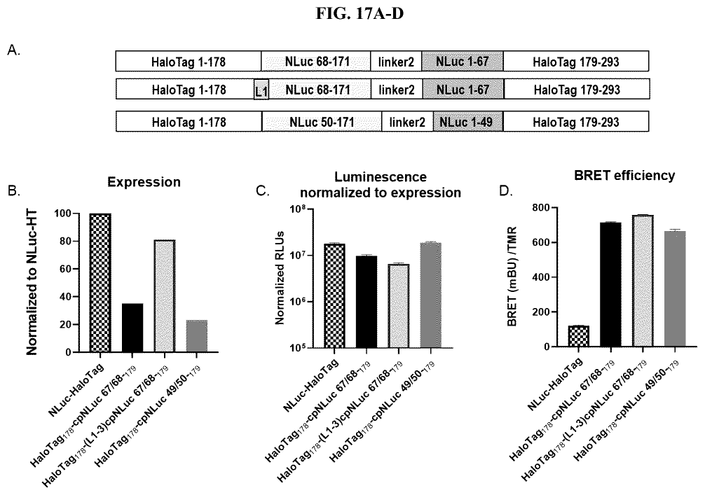

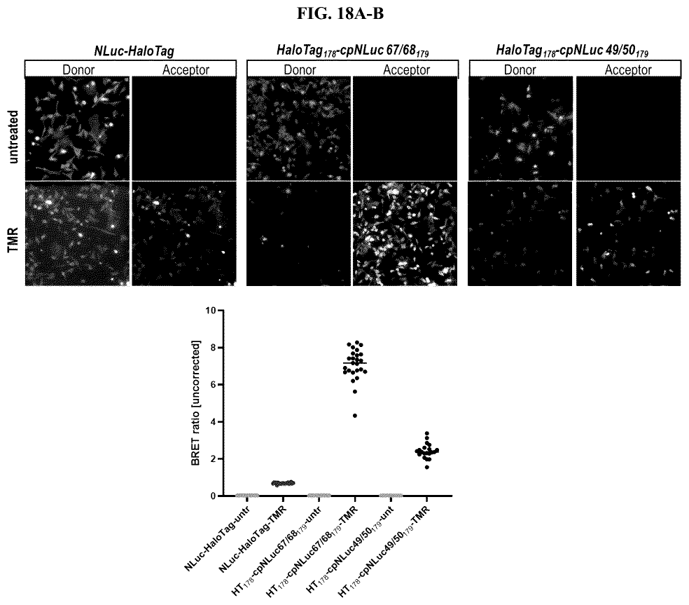

During development of the embodiments described herein, experiments were conducted to further characterize a lead HALOTAG-cpNANOLUC chimera emerging from the screens for alternative circular permutation sites in NanoLuc, which were inserted into HaloTag's loop 180 (i.e., HaloTag₁₇₈-cpNLuc-₁₇₉), and flexible linkers that could be incorporated between chimera's components (FIGS. 16-18). The structures of these chimeras incorporating NanoLuc circularly permuted between either amino acids 67/68 or 49/50 as well as a flexible linker comprising 3 Glycine-Serine residues are described in FIGS. 16 A and 17A. Purified chimeras were compared for their binding kinetics of a HaloTag-TMR ligand, brightness, and efficiency of intramolecular BRET to a bound TMR ligand (FIG. 16). This evaluation revealed that the chimera incorporating cpNLuc 49/50 exhibited faster binding kinetics and was brighter while chimera incorporating cpNLuc 67/68 offered greater BRET efficiency. Furthermore, the binding kinetics of the chimera incorporating cpNLuc 67/68 could be slightly increased by the addition of a flexible linker (L1). Cell-based evaluations of the same chimeras transiently expressed in HeLa cells (FIG. 17) revealed lower expression for the chimeras compared to a NanoLuc-HaloTag fusion. Among the chimeras, chimera incorporating cpNLuc 49/50 had the lower expression. Chimera incorporating cpNLuc 67/68 had higher expression, which was further increased by the addition of flexible linker L1. In agreement with the biochemical evaluation, bioluminescence normalized to expression suggested that chimera incorporating cpNLuc 49/50 is brighter but exhibits lower BRET efficiency to a bound TMR ligand. This was further demonstrated in BRET imaging experiments (FIG. 18) showing that the chimeras, especially the one incorporating cpNLuc 67/68 offers a significantly high BRET efficiency to a bound TMR ligand.

### Example 12

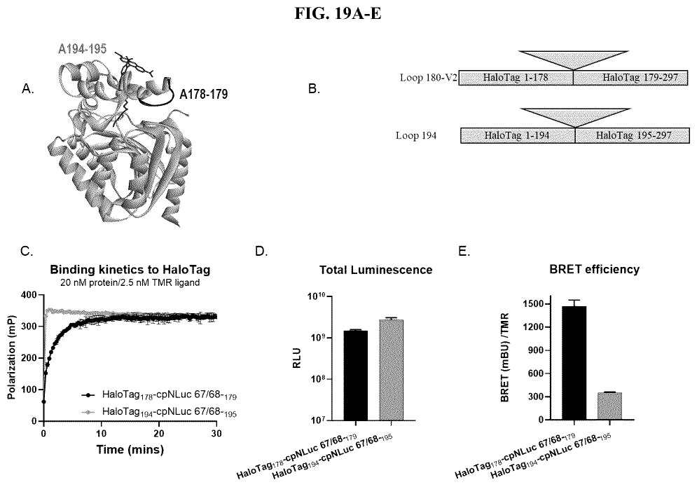

During the development of the embodiments described herein, experiments were conducted to evaluate the capacity of loop 194 to tolerate large insertions. To this end, purified HALOTAG-cpNANOLUC chimeras comprising insertion of cpNanoLuc 67/68 into HaloTag's surface loops 194 and 180 were compared for their binding kinetics of a HaloTag-TMR ligand, brightness, and efficiency of intramolecular BRET to a bound TMR ligand (FIG. 19). This evaluation revealed that loop 194 can tolerate large insertions. Furthermore, the resulting chimera exhibited faster binding kinetics and was brighter compared to chimera generated by insertion into loop 180. At the same time, BRET efficiency was significantly lower. These results further support the highly efficient BRET attributes offered by insertions of circularly permuted NanoLuc into loop 180 presumably through closer proximity and/or conformations more favorable for energy transfer to a bound TMR ligand.

### Example 13

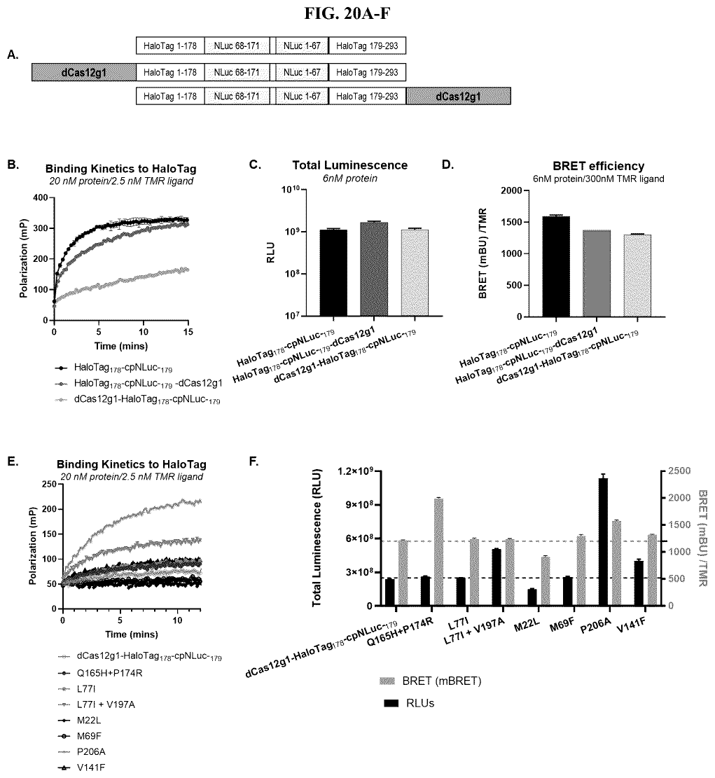

During the development of the embodiments described herein, experiments were conducted to evaluate the tolerance of the HALOTAG-cpNANOLUC chimera to genetic fusions as well as incorporation of additional mutations in the HaloTag domains (FIG. 20). Genetic fusion of the chimera to the N or C terminus of a model protein dCas12g1 were not only successfully expressed and purified from E. coli but also exhibited comparable brightness and BRET efficiencies to those of unfused chimera suggesting the chimera can generally tolerate either N or C terminal fusions. Given the relatively slower binding kinetics of dCas12g1-HaloTag₁₇₈-cpNLuc-₁₇₉ fusion, it was chosen as the template for incorporating additional mutations into the HaloTag domains. Evaluation of purified fusion variants for brightness, BRET efficiencies and binding kinetic of a HaloTag-TMR ligand revealed that these mutations had either no impact or positive impact on the dCas12g1-HaloTag₁₇₈-cpNLuc-₁₇₉ fusion. Two variants incorporating either L77I+V197A or P206A mutations exhibited significant increase in brightness and binding kinetics of a HaloTag-TMR ligand suggesting an overall increased stability without compromising the capacity to adopt a conformation favorable for efficient BRET. Altogether these results further demonstrate the flexible nature of the chimera tolerating different composition of elements.

### Example 14

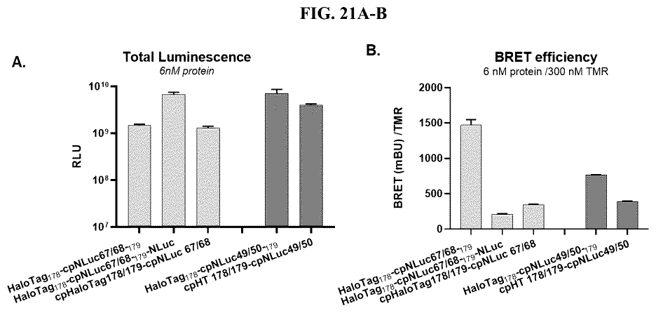

During the development of the embodiments described herein, experiments were conducted to evaluate the properties of different configurations incorporating circularly permuted NLucs either as insertions into HaloTag's loop-180 (i.e., HALOTAG-cpNANOLUC chimera) or fusions to a HaloTag, which was circularly permuted at the same loop (FIG. 21). Biochemical evaluation of the two leading circular permutation sites cpNLuc 67/68 and cpNLuc 49/50 as chimeras or fusions to epHaloTag revealed that configurations incorporating the same cpNLuc had similar brightness. However, the chimera configurations exhibited significant greater BRET efficiencies, presumably through closer proximity and/or conformations more favorable for energy transfer to a bound TMR ligand.

In addition, as expected a genetic fusion of the HALOTAG-cpNANOLUC chimera to NanoLuc resulted in increased brightness. However, this configuration exhibited a significantly smaller increase in BRET efficiency relative to a NanoLuc-HaloTag fusion.

### Example 15

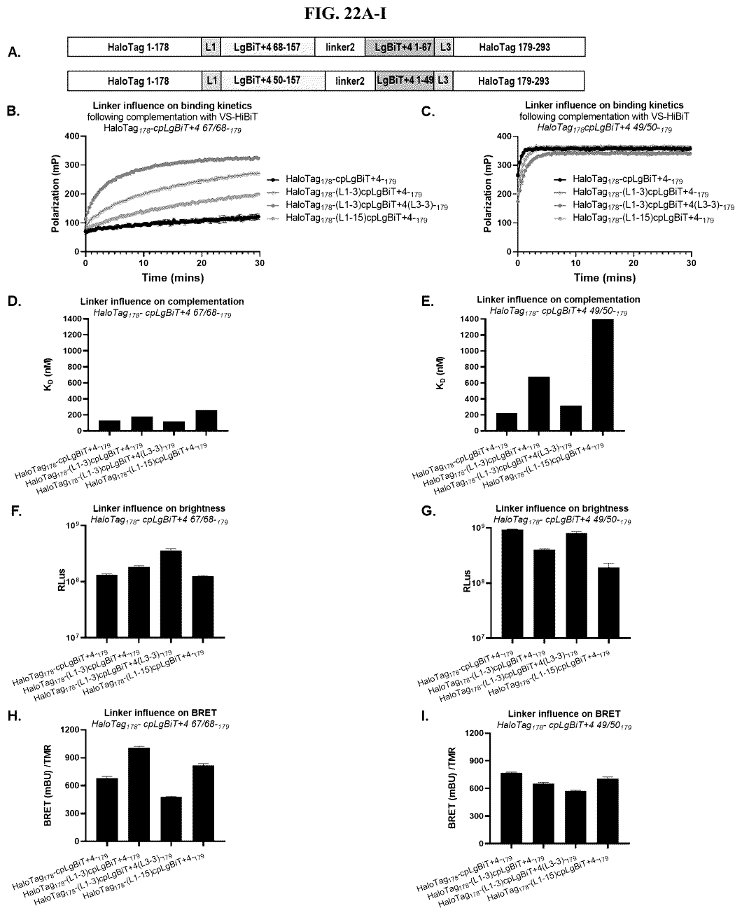

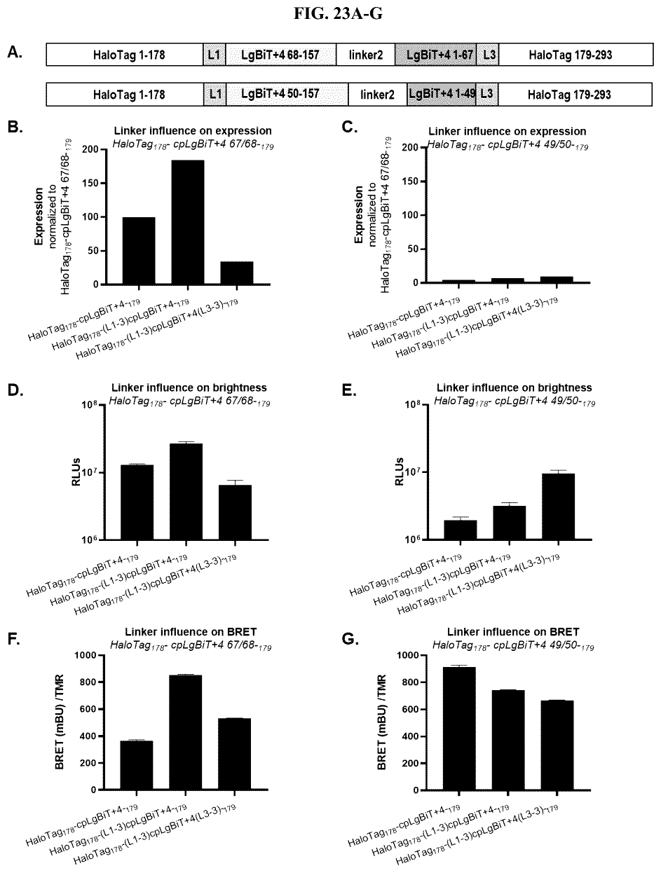

During the development of the embodiments described herein, experiments were conducted to optimize the properties of complementation-based chimeras through circular permutation of LgBiT+4 at the two leading cp sites 67/68 and 49/50 as well as incorporation of flexible Glycine-Serine linkers of different length between components of the chimera (FIGS. 22 and 23). Biochemical evaluation of these chimeras (FIG. 22) revealed that upon complementation with a VS-HiBiT peptide, chimeras incorporating cpLgBiT+4 49/50 exhibited faster binding kinetics of a HaloTag-TMR ligand as well as increased brightness but all together lower affinities to the VS-HiBiT peptide. Incorporation of flexible linkers into HT-17scpLgBiT+4 49/50-179 had no impact or small impact on binding kinetics, brightness, or BRET. However, these linkers, especially the long L1-15 significantly decreased the affinity of HT-17scpLgBiT+4 49/50-179 to VS-HiBiT. A similar analysis for HT-17scpLgBiT+4 67/68-179, revealed that flexible linkers, especially short ones, improved the binding kinetics, brightness, and BRET presumably through greater flexibility and capacity to adopt upon complementation a more stable conformation.

Cell-based evaluations of the same chimeras transfected into genome-edited HeLa cells expressing a HiBiT-tagged-GAPDH revealed significantly lower expression for the chimeras incorporating cpLgBiT+4 49/50 (FIG. 23). Interestingly, evaluations in mammalian cells revealed for both HALOTAG-cpNANOLUC and HALOTAG-cpLGBIT chimeras a preference for incorporation of either cpNLuc or cpLgBiT+4 that were circularly permuted between residues 67/68 as well as additional inclusion of a flexible short L1-3 linker.

### Example 16

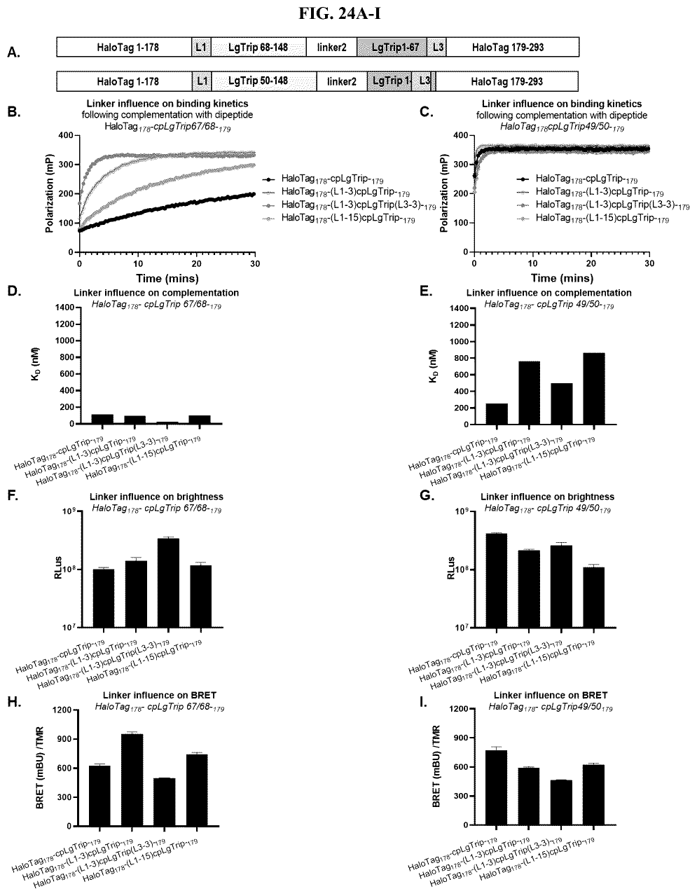

During the development of the embodiments described herein, experiments were conducted to optimize the properties of complementation-based chimeras through replacement of circularly permuted LgBiT+4 with a more stable circularly permuted LgTrip. Same as example 15, the inserted LgTrip was circularly permuted at the two leading cp sites 67/68 and 49/50 and the influence of flexible Glycine-Serine linkers between components of the chimera was further explored (FIG. 24). Biochemical evaluation of these chimeras revealed that upon complementation with a VS-HiBiT-Trip9 dipeptide, chimeras incorporating cpLgTrip 49/50 exhibited faster binding kinetics of a HaloTag-TMR ligand as well as increased brightness but all together lower affinities to the dipeptide. Incorporation of flexible linkers into HT-17scpLgTrip 49/50-179 had no impact on binding kinetics but generally decreased brightness, BRET, and especially binding affinity to the dipeptide. A similar analysis for HT-17scpLgTrip+4 67/68-179, revealed that flexible linkers, especially short ones, improved the binding kinetics, brightness, and BRET presumably through greater flexibility and capacity to adopt upon complementation a more stable conformation.

### Example 17

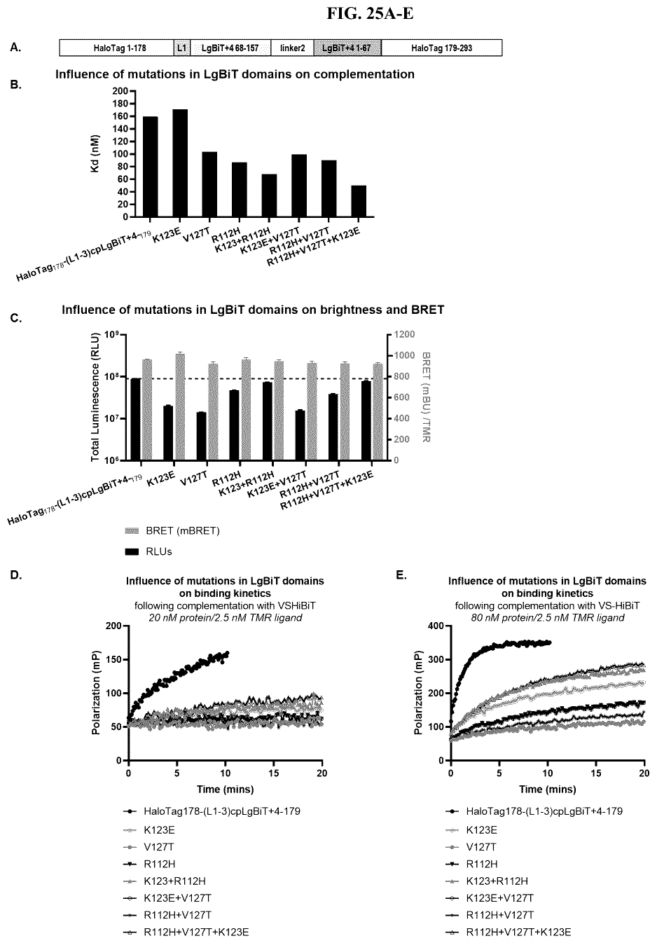

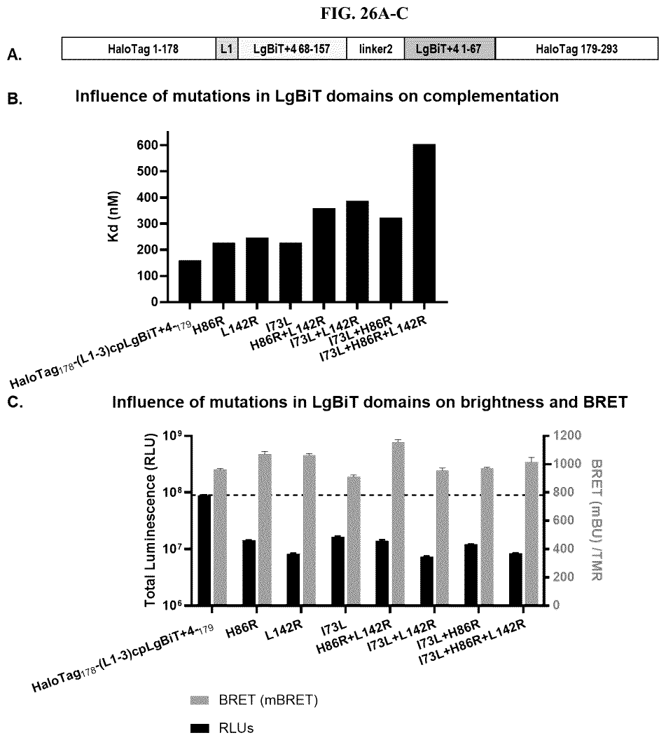

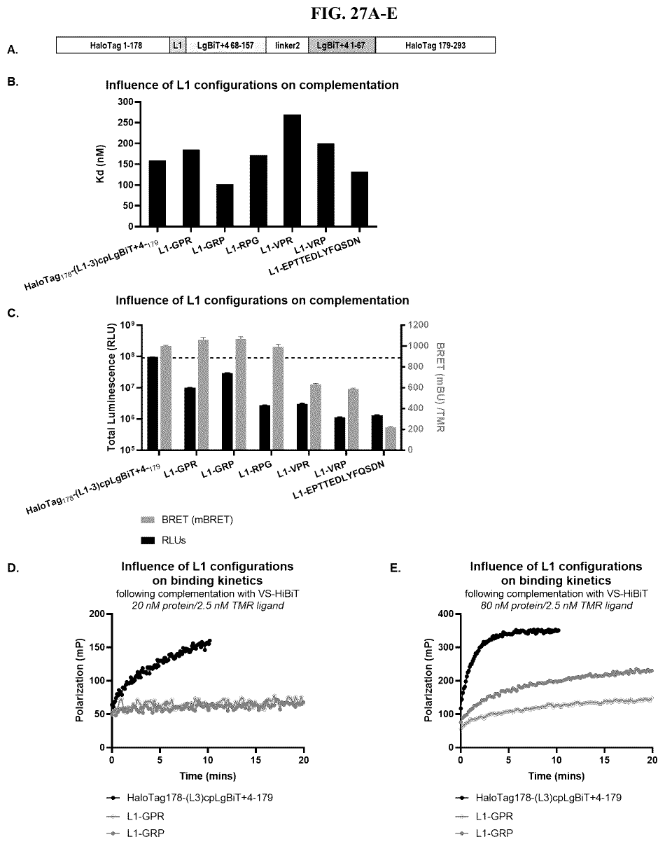

During the development of the embodiments described herein, experiments were conducted to evaluate the tolerance of complementation-based chimeras to incorporation of additional mutations as well as linkers of different nature and lengths. Since among the tested HALOTAG-cpLGBIT chimeras, HT-178(L1-3)cpLgBiT+4 67/68-179 chimera exhibited in mammalian cells the highest expression, brightness, and BRET efficiency, it was chosen as a template for incorporating additional mutations within the LgBiT domains as wells a different configurations of linker L-1 (FIGS. 25-27). The capacity to not only express and purify all these configurations but also to obtain BRET efficiencies that were generally higher than those obtained with a LgBiT-HaloTag fusion demonstrated the flexible nature of these complementation-based chimeras and their compatibility with different configurations. Notably, all the chimeras incorporating the additional LgTrip mutations exhibited a range of higher binding affinities to VS-HiBiT. In particular, the variant incorporating all three additional mutations (R112H+V127T+K123E) exhibited brightness and BRET efficiency that were similar to HT-₁₇₈(L1-3)cpLgBiT+4 67/68-₁₇₉ but a significantly higher affinity to VS-HIBiT.

SEQUENCES
HT
SEQ ID NO: 1
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLNPVDREPLWRFPNELPIAGEPANIVALVEEY
MDWLHQSPVPKLLFWGTPGVLIPPAEAARLAKSLPNCKAVDIGPG
LNLLQEDNPDLIGSEIARWLSTLEISG
HT(loop 165 insertion)
SEQ ID NO: 2
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDX₁X₂X₃X₄X₅X₆X₇X₈
X₉X₁₀X₁₁X₁₂X₁₃X₁₄X₁₅X₁₆X₁₇X₁₈X₁₉X₂₀X₂₁X₂₂X₂₃X₂₄
X₂₅VFIEGTLPMGVVRPLTEVEMDHYREPFLNPVDREPLWRFP
NELPIAGEPANIVALVEEYMDWLHQSPVPKLLFWGTPGVLIPPAE
AARLAKSLPNCKAVDIGPGLNLLQEDNPDLIGSEIARWLSTLEIS
G
HT(position 165 insertion)
SEQ ID NO: 3
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIX₁X₂X₃X₄X₅X₆X₇X₈X₉
X₁₀X₁₁X₁₂X₁₃X₁₄X₁₅X₁₆X₁₇X₁₈X₁₉X₂₀X₂₁X₂₂X₂₃X₂₄
X₂₅VFIEGTLPMGVVRPLTEVEMDHYREPFLNPVDREPLWRFPN
ELPIAGEPANIVALVEEYMDWLHQSPVPKLLFWGTPGVLIPPAEA
ARLAKSLPNCKAVDIGPGLNLLQEDNPDLIGSEIARWLSTLEISG
HT(loop 180 insertion)
SEQ ID NO: 4
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVX₁
X₂X₃X₄X₅X₆X₇X₈X₉X₁₀X₁₁X₁₂X₁₃X₁₄X₁₅X₁₆X₁₇X₁₈X₁₉
X₂₀X₂₁X₂₂X₂₃X₂₄X₂₅RPLTEVEMDHYREPFLNPVDREPLWR
FPNELPIAGEPANIVALVEEYMDWLHQSPVPKLLFWGTPGVLIPP
AEAARLAKSLPNCKAVDIGPGLNLLQEDNPDLIGSEIARWLSTLE
ISG
HT(position 180 insertion)
SEQ ID NO: 5
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
X₁X₂X₃X₄X₅X₆X₇X₈X₉X₁₀X₁₁X₁₂X₁₃X₁₄X₁₅X₁₆X₁₇X₁₈
X₁₉X₂₀X₂₁X₂₂X₂₃X₂₄X₂₅LTEVEMDHYREPFLNPVDREPLWR
FPNELPIAGEPANIVALVEEYMDWLHQSPVPKLLFWGTPGVLIPP
AEAARLAKSLPNCKAVDIGPGLNLLQEDNPDLIGSEIARWLSTLE
ISG
HT(1-163)
SEQ ID NO: 6
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLII
HT(1-164)
SEQ ID NO: 7
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIID
HT(1-165)
SEQ ID NO: 8
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQ
HT(1-166)
SEQ ID NO: 9
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQN
HT(164-297)
SEQ ID NO: 10
DQNVFIEGTLPMGVVRPLTEVEMDHYREPFLNPVDREPLWRFPNE
LPIAGEPANIVALVEEYMDWLHQSPVPKLLFWGTPGVLIPPAEAA
RLAKSLPNCKAVDIGPGLNLLQEDNPDLIGSEIARWLSTLEISG
HT(165-297)
SEQ ID NO: 11
QNVFIEGTLPMGVVRPLTEVEMDHYREPFLNPVDREPLWRFPNEL
PIAGEPANIVALVEEYMDWLHQSPVPKLLFWGTPGVLIPPAEAAR
LAKSLPNCKAVDIGPGLNLLQEDNPDLIGSEIARWLSTLEISG
HT(166-297)
SEQ ID NO: 12
NVFIEGTLPMGVVRPLTEVEMDHYREPFLNPVDREPLWRFPNELP
IAGEPANIVALVEEYMDWLHQSPVPKLLFWGTPGVLIPPAEAARL
AKSLPNCKAVDIGPGLNLLQEDNPDLIGSEIARWLSTLEISG
HT(167-297)
SEQ ID NO: 13
VFIEGTLPMGVVRPLTEVEMDHYREPFLNPVDREPLWRFPNELPI
AGEPANIVALVEEYMDWLHQSPVPKLLFWGTPGVLIPPAEAARLA
KSLPNCKAVDIGPGLNLLQEDNPDLIGSEIARWLSTLEISG
HT(1-176)
SEQ ID NO: 14
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMG
HT(1-177)
SEQ ID NO: 15
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGV
HT(1-178)
SEQ ID NO: 16
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVV
HT(1-179)
SEQ ID NO: 17
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVR
HT(1-180)
SEQ ID NO: 18
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
HT(1-181)
SEQ ID NO: 19
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRPL
HT(1-182)
SEQ ID NO: 20
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LT
HT(177-297)
SEQ ID NO: 21
VVRPLTEVEMDHYREPFLNPVDREPLWRFPNELPIAGEPANIVAL
VEEYMDWLHQSPVPKLLFWGTPGVLIPPAEAARLAKSLPNCKAVD
IGPGLNLLQEDNPDLIGSEIARWLSTLEISG
HT(178-297)
SEQ ID NO: 22
VRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HT(179-297)
SEQ ID NO: 23
RPLTEVEMDHYREPFLNPVDREPLWRFPNELPIAGEPANIVALVE
EYMDWLHQSPVPKLLFWGTPGVLIPPAEAARLAKSLPNCKAVDIG
PGLNLLQEDNPDLIGSEIARWLSTLEISG
HT(180-297)
SEQ ID NO: 24
PLTEVEMDHYREPFLNPVDREPLWRFPNELPIAGEPANIVALVEE
YMDWLHQSPVPKLLFWGTPGVLIPPAEAARLAKSLPNCKAVDIGP
GLNLLQEDNPDLIGSEIARWLSTLEISG
HT(181-297)
SEQ ID NO: 25
LTEVEMDHYREPFLNPVDREPLWRFPNELPIAGEPANIVALVEEY
MDWLHQSPVPKLLFWGTPGVLIPPAEAARLAKSLPNCKAVDIGPG
LNLLQEDNPDLIGSEIARWLSTLEISG
HT(182-297)
SEQ ID NO: 26
TEVEMDHYREPFLNPVDREPLWRFPNELPIAGEPANIVALVEEYM
DWLHQSPVPKLLFWGTPGVLIPPAEAARLAKSLPNCKAVDIGPGL
NLLQEDNPDLIGSEIARWLSTLEISG
HT(183-297)
SEQ ID NO: 27
EVEMDHYREPFLNPVDREPLWRFPNELPIAGEPANIVALVEEYMD
WLHQSPVPKLLFWGTPGVLIPPAEAARLAKSLPNCKAVDIGPGLN
LLQEDNPDLIGSEIARWLSTLEISG
NANOLUC
SEQ ID NO: 28
MKHHHHHHAIAMVFTLEDFVGDWRQTAGYNLDQVLEQGGVSSLFQ
NLGVSVTPIQRIVLSGENGLKIDIHVIIPYEGLSGDQMGQIEKIF
KVVYPVDDHHFKVILHYGTLVIDGVTPNMIDYFGRPYEGIAVFDG
KKITVTGTLWNGNKIIDERLINPDGSLLFRVTINGVTGWRLCERI
LAV
LgBiT
SEQ ID NO: 29
MVFTLEDFVGDWEQTAAYNLDQVLEQGGVSSLLQNLAVSVTPIQR
IVRSGENALKIDIHVIIPYEGLSADQMAQIEEVFKVVYPVDDHHF
KVILPYGTLVIDGVTPNMLNYFGRPYEGIAVFDGKKITVTGTLWN
GNKIIDERLITPDGSMLFRVTINSHHHHHH
SmBIT
SEQ ID NO: 30
VTGYRLFEEIL
HiBIT
SEQ ID NO: 31
VSGWRLFKKIS
Dual insert sequence
SEQ ID NO: 32
NVFIEGTLPMG

Exemplary Circularly Permuted NanoLuc Inserts into HaloTag Loops
Nomenclature: “HaloTag[HT residue preceding the insert]-cpNLuc[NLuc residue preceding CP site/NLuc residue following CP site]-[HT residue following the insert]”
* all constructs contain a linker between the two NLuc domains; constructs may also contain one or more linkers

HaloTag164-cpNLuc67/68-167
SEQ ID NO: 33
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDSGDQMGQIEKIFKVVY
PVDDHHFKVILHYGTLVIDGVTPNMIDYFGRPYEGIAVFDGKKIT
VTGTLWNGNKIIDERLINPDGSLLFRVTINGVTGWRLCERILAGG
TGGSGGTGGSMVFTLEDFVGDWRQTAGYNLDQVLEQGGVSSLFQN
LGVSVTPIQRIVLSGENGLKIDIHVIIPYEGLVFIEGTLPMGVVR
PLTEVEMDHYREPFLNPVDREPLWRFPNELPIAGEPANIVALVEE
YMDWLHQSPVPKLLFWGTPGVLIPPAEAARLAKSLPNCKAVDIGP
GLNLLQEDNPDLIGSEIARWLSTLEISG
HaloTag194-cpNLuc67/68-195
SEQ ID NO: 34
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLSGDQMGQIEKIFKVVYPVDDHHFKVILHYGT
LVIDGVTPNMIDYFGRPYEGIAVFDGKKITVTGTLWNGNKIIDER
LINPDGSLLFRVTINGVTGWRLCERILAGGTGGSGGTGGSMVFTL
EDFVGDWRQTAGYNLDQVLEQGGVSSLFQNLGVSVTPIQRIVLSG
ENGLKIDIHVIIPYEGLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
Halo Tag178-NLuc-179
SEQ ID NO: 35
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVMV
FTLEDFVGDWRQTAGYNLDQVLEQGGVSSLFQNLGVSVTPIQRIV
LSGENGLKIDIHVIIPYEGLSGDQMGQIEKIFKVVYPVDDHHFKV
ILHYGTLVIDGVTPNMIDYFGRPYEGIAVFDGKKITVTGTLWNGN
KIIDERLINPDGSLLFRVTINGVTGWRLCERILARPLTEVEMDHY
REPFLNPVDREPLWRFPNELPIAGEPANIVALVEEYMDWLHQSPV
PKLLFWGTPGVLIPPAEAARLAKSLPNCKAVDIGPGLNLLQEDNP
DLIGSEIARWLSTLEISG
Halo Tag178-cpNLuc67/68-179
SEQ ID NO: 36
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
Halo Tag178-cptsNLuc67/68-179
SEQ ID NO: 37
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSA
DQMAQIEEVFKVVYPVDDHHFKVILPYGTLVIDGVTPNKLNYFGH
PYEGIAVFDGEKITVTGTLWNGNKIIDERLITPDGSMLFRVTING
VSGWRLFKKISGGTGGSGGTGGSMVFTLDDFVGDWEQTAAYNLDQ
VLEQGGVSSLLQNLAVSVTPIQRIVRSGENALKIDIHVIIPYEGL
RPLTEVEMDHYREPFLNPVDREPLWRFPNELPIAGEPANIVALVE
EYMDWLHQSPVPKLLFWGTPGVLIPPAEAARLAKSLPNCKAVDIG
PGLNLLQEDNPDLIGSEIARWLSTLEISG
HaloTag178-cptsNLuc67/68-179
SEQ ID NO: 38
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSA
DQMAQIEEVFKVVYPVDDHHFKVILPYGTLVIDGVTPNKLNYFGH
PYEGIAVFDGEKITVTGTLWNGNKIIDERLITPDGSMLFRVTING
VSGWRLCKKISGGTGGSGGTGGSMVFTLDDFVGDWEQTAAYNLDQ
VLEQGGVSSLLQNLAVSVTPIQRIVRSGENALKIDIHVIIPYEGL
RPLTEVEMDHYREPFLNPVDREPLWRFPNELPIAGEPANIVALVE
EYMDWLHQSPVPKLLFWGTPGVLIPPAEAARLAKSLPNCKAVDIG
PGLNLLQEDNPDLIGSEIARWLSTLEISG
Halo Tag178-cpNLuc6/7-179
SEQ ID NO: 39
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVDF
VGDWRQTAGYNLDQVLEQGGVSSLFQNLGVSVTPIQRIVLSGENG
LKIDIHVIIPYEGLSGDQMGQIEKIFKVVYPVDDHHFKVILHYGT
LVIDGVTPNMIDYFGRPYEGIAVFDGKKITVTGTLWNGNKIIDER
LINPDGSLLFRVTINGVTGWRLCERILAGGTGGSGGTGGSMVFTL
ERPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc12/13-179
SEQ ID NO: 40
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRQ
TAGYNLDQVLEQGGVSSLFQNLGVSVTPIQRIVLSGENGLKIDIH
VIIPYEGLSGDQMGQIEKIFKVVYPVDDHHFKVILHYGTLVIDGV
TPNMIDYFGRPYEGIAVFDGKKITVTGTLWNGNKIIDERLINPDG
SLLFRVTINGVTGWRLCERILAGGTGGSGGTGGSMVFTLEDFVGD
WRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc24/25-179
SEQ ID NO: 41
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVEQ
GGVSSLFQNLGVSVTPIQRIVLSGENGLKIDIHVIIPYEGLSGDQ
MGQIEKIFKVVYPVDDHHFKVILHYGTLVIDGVTPNMIDYFGRPY
EGIAVFDGKKITVTGTLWNGNKIIDERLINPDGSLLFRVTINGVT
GWRLCERILAGGTGGSGGTGGSMVFTLEDFVGDWRQTAGYNLDQV
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc27/28-179
SEQ ID NO: 42
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGV
SSLFQNLGVSVTPIQRIVLSGENGLKIDIHVIIPYEGLSGDQMGQ
IEKIFKVVYPVDDHHFKVILHYGTLVIDGVTPNMIDYFGRPYEGI
AVFDGKKITVTGTLWNGNKIIDERLINPDGSLLFRVTINGVTGWR
LCERILAGGTGGSGGTGGSMVFTLEDFVGDWRQTAGYNLDQVLEQ
GRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc49/50-179
SEQ ID NO: 43
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGE
NGLKIDIHVIIPYEGLSGDQMGQIEKIFKVVYPVDDHHFKVILHY
GTLVIDGVTPNMIDYFGRPYEGIAVFDGKKITVTGTLWNGNKIID
ERLINPDGSLLFRVTINGVTGWRLCERILAGGTGGSGGTGGSMVF
TLEDFVGDWRQTAGYNLDQVLEQGGVSSLFQNLGVSVTPIQRIVL
SRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc52/53-179
SEQ ID NO: 44
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGL
KIDIHVIIPYEGLSGDQMGQIEKIFKVVYPVDDHHFKVILHYGTL
VIDGVTPNMIDYFGRPYEGIAVFDGKKITVTGTLWNGNKIIDERL
INPDGSLLFRVTINGVTGWRLCERILAGGTGGSGGTGGSMVFTLE
DFVGDWRQTAGYNLDQVLEQGGVSSLFQNLGVSVTPIQRIVLSGE
NRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
Halo Tag178-cpNLuc55/56-179
SEQ ID NO: 45
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVID
IHVIIPYEGLSGDQMGQIEKIFKVVYPVDDHHFKVILHYGTLVID
GVTPNMIDYFGRPYEGIAVFDGKKITVTGTLWNGNKIIDERLINP
DGSLLFRVTINGVTGWRLCERILAGGTGGSGGTGGSMVFTLEDFV
GDWRQTAGYNLDQVLEQGGVSSLFQNLGVSVTPIQRIVLSGENGL
KRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc64/65-179
SEQ ID NO: 46
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVEG
LSGDQMGQIEKIFKVVYPVDDHHFKVILHYGTLVIDGVTPNMIDY
FGRPYEGIAVFDGKKITVTGTLWNGNKIIDERLINPDGSLLFRVT
INGVTGWRLCERILAGGTGGSGGTGGSMVFTLEDFVGDWRQTAGY
NLDQVLEQGGVSSLFQNLGVSVTPIQRIVLSGENGLKIDIHVIIP
YRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc70/71-179
SEQ ID NO: 47
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVQM
GQIEKIFKVVYPVDDHHFKVILHYGTLVIDGVTPNMIDYFGRPYE
GIAVFDGKKITVTGTLWNGNKIIDERLINPDGSLLFRVTINGVTG
WRLCERILAGGTGGSGGTGGSMVFTLEDFVGDWRQTAGYNLDQVL
EQGGVSSLFQNLGVSVTPIQRIVLSGENGLKIDIHVIIPYEGLSG
DRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc79/80-179
SEQ ID NO: 48
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVKV
VYPVDDHHFKVILHYGTLVIDGVTPNMIDYFGRPYEGIAVFDGKK
ITVTGTLWNGNKIIDERLINPDGSLLFRVTINGVTGWRLCERILA
GGTGGSGGTGGSMVFTLEDFVGDWRQTAGYNLDQVLEQGGVSSLF
QNLGVSVTPIQRIVLSGENGLKIDIHVIIPYEGLSGDQMGQIEKI
FRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
Halo Tag178-cpNLuc82/83-179
SEQ ID NO: 49
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVYP
VDDHHFKVILHYGTLVIDGVTPNMIDYFGRPYEGIAVFDGKKITV
TGTLWNGNKIIDERLINPDGSLLFRVTINGVTGWRLCERILAGGT
GGSGGTGGSMVFTLEDFVGDWRQTAGYNLDQVLEQGGVSSLFQNL
GVSVTPIQRIVLSGENGLKIDIHVIIPYEGLSGDQMGQIEKIFKV
VRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc84/85-179
SEQ ID NO: 50
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVVD
DHHFKVILHYGTLVIDGVTPNMIDYFGRPYEGIAVFDGKKITVTG
TLWNGNKIIDERLINPDGSLLFRVTINGVTGWRLCERILAGGTGG
SGGTGGSMVFTLEDFVGDWRQTAGYNLDQVLEQGGVSSLFQNLGV
SVTPIQRIVLSGENGLKIDIHVIIPYEGLSGDQMGQIEKIFKVVY
PRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc86/87-179
SEQ ID NO: 51
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVDH
HFKVILHYGTLVIDGVTPNMIDYFGRPYEGIAVFDGKKITVTGTL
WNGNKIIDERLINPDGSLLFRVTINGVTGWRLCERILAGGTGGSG
GTGGSMVFTLEDFVGDWRQTAGYNLDQVLEQGGVSSLFQNLGVSV
TPIQRIVLSGENGLKIDIHVIIPYEGLSGDQMGQIEKIFKVVYPV
DRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc103/104-179
SEQ ID NO: 52
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVVT
PNMIDYFGRPYEGIAVFDGKKITVTGTLWNGNKIIDERLINPDGS
LLFRVTINGVTGWRLCERILAGGTGGSGGTGGSMVFTLEDFVGDW
RQTAGYNLDQVLEQGGVSSLFQNLGVSVTPIQRIVLSGENGLKID
IHVIIPYEGLSGDQMGQIEKIFKVVYPVDDHHFKVILHYGTLVID
GRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc106/107-179
SEQ ID NO: 53
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVNM
IDYFGRPYEGIAVFDGKKITVTGTLWNGNKIIDERLINPDGSLLF
RVTINGVTGWRLCERILAGGTGGSGGTGGSMVFTLEDFVGDWRQT
AGYNLDQVLEQGGVSSLFQNLGVSVTPIQRIVLSGENGLKIDIHV
IIPYEGLSGDQMGQIEKIFKVVYPVDDHHFKVILHYGTLVIDGVT
PRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc120/121-179
SEQ ID NO: 54
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVVF
DGKKITVTGTLWNGNKIIDERLINPDGSLLFRVTINGVTGWRLCE
RILAGGTGGSGGTGGSMVFTLEDFVGDWRQTAGYNLDQVLEQGGV
SSLFQNLGVSVTPIQRIVLSGENGLKIDIHVIIPYEGLSGDQMGQ
IEKIFKVVYPVDDHHFKVILHYGTLVIDGVTPNMIDYFGRPYEGI
ARPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc124/125-179
SEQ ID NO: 55
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVKK
ITVTGTLWNGNKIIDERLINPDGSLLFRVTINGVTGWRLCERILA
GGTGGSGGTGGSMVFTLEDFVGDWRQTAGYNLDQVLEQGGVSSLF
QNLGVSVTPIQRIVLSGENGLKIDIHVIIPYEGLSGDQMGQIEKI
FKVVYPVDDHHFKVILHYGTLVIDGVTPNMIDYFGRPYEGIAVFD
GRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc130/131-179
SEQ ID NO: 56
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGT
LWNGNKIIDERLINPDGSLLFRVTINGVTGWRLCERILAGGTGGS
GGTGGSMVFTLEDFVGDWRQTAGYNLDQVLEQGGVSSLFQNLGVS
VTPIQRIVLSGENGLKIDIHVIIPYEGLSGDQMGQIEKIFKVVYP
VDDHHFKVILHYGTLVIDGVTPNMIDYFGRPYEGIAVFDGKKITV
TRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc145/146-179
SEQ ID NO: 57
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVNP
DGSLLFRVTINGVTGWRLCERILAGGTGGSGGTGGSMVFTLEDFV
GDWRQTAGYNLDQVLEQGGVSSLFQNLGVSVTPIQRIVLSGENGL
KIDIHVIIPYEGLSGDQMGQIEKIFKVVYPVDDHHFKVILHYGTL
VIDGVTPNMIDYFGRPYEGIAVFDGKKITVTGTLWNGNKIIDERL
IRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-cpNLuc148/149-179
SEQ ID NO: 58
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGS
LLFRVTINGVTGWRLCERILAGGTGGSGGTGGSMVFTLEDFVGDW
RQTAGYNLDQVLEQGGVSSLFQNLGVSVTPIQRIVLSGENGLKID
IHVIIPYEGLSGDQMGQIEKIFKVVYPVDDHHFKVILHYGTLVID
GVTPNMIDYFGRPYEGIAVFDGKKITVTGTLWNGNKIIDERLINP
DRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-(L1-3)cpNLuc67/68-179
SEQ ID NO: 59
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSGDQMGQIEKIFKVVYPVDDHHFKVILHYGTLVIDGVTPNMIDY
FGRPYEGIAVFDGKKITVTGTLWNGNKIIDERLINPDGSLLFRVT
INGVTGWRLCERILAGGTGGSGGTGGSMVFTLEDFVGDWRQTAGY
NLDQVLEQGGVSSLFQNLGVSVTPIQRIVLSGENGLKIDIHVIIP
YEGLRPLTEVEMDHYREPFLNPVDREPLWRFPNELPIAGEPANIV
ALVEEYMDWLHQSPVPKLLFWGTPGVLIPPAEAARLAKSLPNCKA
VDIGPGLNLLQEDNPDLIGSEIARWLSTLEISG
HaloTag178-(L1-6)cpNLuc67/68-179
SEQ ID NO: 60
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGSGDQMGQIEKIFKVVYPVDDHHFKVILHYGTLVIDGVTPNM
IDYFGRPYEGIAVFDGKKITVTGTLWNGNKIIDERLINPDGSLLF
RVTINGVTGWRLCERILAGGTGGSGGTGGSMVFTLEDFVGDWRQT
AGYNLDQVLEQGGVSSLFQNLGVSVTPIQRIVLSGENGLKIDIHV
IIPYEGLRPLTEVEMDHYREPFLNPVDREPLWRFPNELPIAGEPA
NIVALVEEYMDWLHQSPVPKLLFWGTPGVLIPPAEAARLAKSLPN
CKAVDIGPGLNLLQEDNPDLIGSEIARWLSTLEISG
HaloTag178-(L1-9)cpNLuc67/68-179
SEQ ID NO: 61
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGSGDQMGQIEKIFKVVYPVDDHHFKVILHYGTLVIDGVT
PNMIDYFGRPYEGIAVFDGKKITVTGTLWNGNKIIDERLINPDGS
LLFRVTINGVTGWRLCERILAGGTGGSGGTGGSMVFTLEDFVGDW
RQTAGYNLDQVLEQGGVSSLFQNLGVSVTPIQRIVLSGENGLKID
IHVIIPYEGLRPLTEVEMDHYREPFLNPVDREPLWRFPNELPIAG
EPANIVALVEEYMDWLHQSPVPKLLFWGTPGVLIPPAEAARLAKS
LPNCKAVDIGPGLNLLQEDNPDLIGSEIARWLSTLEISG
HaloTag178-(L1-12)cpNLuc67/68-179
SEQ ID NO: 62
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGGSSSGDQMGQIEKIFKVVYPVDDHHFKVILHYGTLVID
GVTPNMIDYFGRPYEGIAVFDGKKITVTGTLWNGNKIIDERLINP
DGSLLFRVTINGVTGWRLCERILAGGTGGSGGTGGSMVFTLEDFV
GDWRQTAGYNLDQVLEQGGVSSLFQNLGVSVTPIQRIVLSGENGL
KIDIHVIIPYEGLRPLTEVEMDHYREPFLNPVDREPLWRFPNELP
IAGEPANIVALVEEYMDWLHQSPVPKLLFWGTPGVLIPPAEAARL
AKSLPNCKAVDIGPGLNLLQEDNPDLIGSEIARWLSTLEISG
HaloTag178-(L1-15)cpNLuc67/68-179
SEQ ID NO: 63
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGGSSSGGSGDQMGQIEKIFKVVYPVDDHHFKVILHYGTL
VIDGVTPNMIDYFGRPYEGIAVFDGKKITVTGTLWNGNKIIDERL
INPDGSLLFRVTINGVTGWRLCERILAGGTGGSGGTGGSMVFTLE
DFVGDWRQTAGYNLDQVLEQGGVSSLFQNLGVSVTPIQRIVLSGE
NGLKIDIHVIIPYEGLRPLTEVEMDHYREPFLNPVDREPLWRFPN
ELPIAGEPANIVALVEEYMDWLHQSPVPKLLFWGTPGVLIPPAEA
ARLAKSLPNCKAVDIGPGLNLLQEDNPDLIGSEIARWLSTLEISG
HaloTag178-(L2-3)cpNLuc67/68-179
SEQ ID NO: 64
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGSMVFTLEDFVGDWRQTAGYNLDQVLEQGGVS
SLFQNLGVSVTPIQRIVLSGENGLKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag178-(L2-6)cpNLuc67/68-179
SEQ ID NO: 65
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGSGGGMVFTLEDFVGDWRQTAGYNLDQVLEQG
GVSSLFQNLGVSVTPIQRIVLSGENGLKIDIHVIIPYEGLRPLTE
VEMDHYREPFLNPVDREPLWRFPNELPIAGEPANIVALVEEYMDW
LHQSPVPKLLFWGTPGVLIPPAEAARLAKSLPNCKAVDIGPGLNL
LQEDNPDLIGSEIARWLSTLEISG
HaloTag178-(L2-9)cpNLuc67/68-179
SEQ ID NO: 66
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGSGGGGSGMVFTLEDFVGDWRQTAGYNLDQVL
EQGGVSSLFQNLGVSVTPIQRIVLSGENGLKIDIHVIIPYEGLRP
LTEVEMDHYREPFLNPVDREPLWRFPNELPIAGEPANIVALVEEY
MDWLHQSPVPKLLFWGTPGVLIPPAEAARLAKSLPNCKAVDIGPG
LNLLQEDNPDLIGSEIARWLSTLEISG
HaloTag178-(L2-12)cpNLuc67/68-179
SEQ ID NO: 67
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGSGGGGSGGSSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178-(L2-15)cpNLuc67/68-179
SEQ ID NO: 68
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGSGGGGSGGSSSGGMVFTLEDFVGDWRQTAGY
NLDQVLEQGGVSSLFQNLGVSVTPIQRIVLSGENGLKIDIHVIIP
YEGLRPLTEVEMDHYREPFLNPVDREPLWRFPNELPIAGEPANIV
ALVEEYMDWLHQSPVPKLLFWGTPGVLIPPAEAARLAKSLPNCKA
VDIGPGLNLLQEDNPDLIGSEIARWLSTLEISG
Halo Tag178-(L3-3)cpNLuc67/68-179
SEQ ID NO: 69
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LGGSRPLTEVEMDHYREPFLNPVDREPLWRFPNELPIAGEPANIV
ALVEEYMDWLHQSPVPKLLFWGTPGVLIPPAEAARLAKSLPNCKA
VDIGPGLNLLQEDNPDLIGSEIARWLSTLEISG
HaloTag178-(L3-6)cpNLuc67/68-179
SEQ ID NO: 70
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LGGSGGGRPLTEVEMDHYREPFLNPVDREPLWRFPNELPIAGEPA
NIVALVEEYMDWLHQSPVPKLLFWGTPGVLIPPAEAARLAKSLPN
CKAVDIGPGLNLLQEDNPDLIGSEIARWLSTLEISG
HaloTag178-(L3-9)cpNLuc67/68-179
SEQ ID NO: 71
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LGGSGGGGSGRPLTEVEMDHYREPFLNPVDREPLWRFPNELPIAG
EPANIVALVEEYMDWLHQSPVPKLLFWGTPGVLIPPAEAARLAKS
LPNCKAVDIGPGLNLLQEDNPDLIGSEIARWLSTLEISG
HaloTag178-(L3-12)cpNLuc67/68-179
SEQ ID NO: 72
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LGGSGGGGSGGSSRPLTEVEMDHYREPFLNPVDREPLWRFPNELP
IAGEPANIVALVEEYMDWLHQSPVPKLLFWGTPGVLIPPAEAARL
AKSLPNCKAVDIGPGLNLLQEDNPDLIGSEIARWLSTLEISG
HaloTag178-(L3-15)cpNLuc67/68-179
SEQ ID NO: 73
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LGGSGGGGSGGSSSGGRPLTEVEMDHYREPFLNPVDREPLWRFPN
ELPIAGEPANIVALVEEYMDWLHQSPVPKLLFWGTPGVLIPPAEA
ARLAKSLPNCKAVDIGPGLNLLQEDNPDLIGSEIARWLSTLEISG
HaloTag178(Q165H+P174R)-cpNLuc67/68179
SEQ ID NO: 74
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDHNVFIEGTLRMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
Halo Tag178 (L771I)-cpNLuc67/68179
SEQ ID NO: 75
GSEIGTGFPFDPHYVEVLGERMHYVDVGPRDGTPVLFLHGNPTSS
YVWRNIIPHVAPTHRCIAPDLIGMGKSDKPDIGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178(L771)-cpNLuc67/68-179(V197A)
SEQ ID NO: 76
GSEIGTGFPFDPHYVEVLGERMHYVDVGPRDGTPVLFLHGNPTSS
YVWRNIIPHVAPTHRCIAPDLIGMGKSDKPDIGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPADREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
Halo Tag178(M22L)-cpNLuc67/68179
SEQ ID NO: 77
GSEIGTGFPFDPHYVEVLGERLHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178(M69F)-cpNLuc67/68-179
SEQ ID NO: 78
GSEIGTGFPFDPHYVEVLGERMHYVDVGPRDGTPVLFLHGNPTSS
YVWRNIIPHVAPTHRCIAPDLIGFGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVEDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178(P206A)-cpNLuc67/68-179
SEQ ID NO: 79
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFANELPIAGEPANIVALV
EEYMDWLHQSPVPKLLFWGTPGVLIPPAEAARLAKSLPNCKAVDI
GPGLNLLQEDNPDLIGSEIARWLSTLEISG
HaloTag178(W141E)-cpNLuc67/68-179
SEQ ID NO: 80
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEFPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISG
HT(1-192)
SEQ ID NO: 81
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREP
HT(1-193)
SEQ ID NO: 82
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPF
HT(1-194)
SEQ ID NO: 83
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFL
HT(1-195)
SEQ ID NO: 84
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLN
HT(1-196)
SEQ ID NO: 85
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLNP
HT(193-297)
SEQ ID NO: 86
FLNPVDREPLWRFPNELPIAGEPANIVALVEEYMDWLHQSPVPKL
LFWGTPGVLIPPAEAARLAKSLPNCKAVDIGPGLNLLQEDNPDLI
GSEIARWLSTLEISG
HT(194-297)
SEQ ID NO: 87
LNPVDREPLWRFPNELPIAGEPANIVALVEEYMDWLHQSPVPKLL
FWGTPGVLIPPAEAARLAKSLPNCKAVDIGPGLNLLQEDNPDLIG
SEIARWLSTLEISG
HT(195-297)
SEQ ID NO: 88
NPVDREPLWRFPNELPIAGEPANIVALVEEYMDWLHQSPVPKLLF
WGTPGVLIPPAEAARLAKSLPNCKAVDIGPGLNLLQEDNPDLIGS
EIARWLSTLEISG
HT(196-297)
SEQ ID NO: 89
PVDREPLWRFPNELPIAGEPANIVALVEEYMDWLHQSPVPKLLFW
GTPGVLIPPAEAARLAKSLPNCKAVDIGPGLNLLQEDNPDLIGSE
IARWLSTLEISG
HT(197-297)
SEQ ID NO: 90
VDREPLWRFPNELPIAGEPANIVALVEEYMDWLHQSPVPKLLFWG
TPGVLIPPAEAARLAKSLPNCKAVDIGPGLNLLQEDNPDLIGSEI
ARWLSTLEISG

Exemplary Circularly Permuted LgBiT Inserts into HaloTag Loops
Nomenclature: “HaloTag[HT residue preceding the insert]-cpLgBiT[LgBiT residue preceding CP site/LgBiT residue following CP site]-[HT residue following the insert]”
* Constructs contain a linker in between the two LgBiT domains; constructs may also contain one or more linkers

HaloTag-178-cpLgBIT 67/68-179
SEQ ID NO: 91
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSA
DQMAQIEEVFKVVYPVDDHHFKVILPYGTLVIDGVTPNMLNYFGR
PYEGIAVFDGKKITVTGTLWNGNKIIDERLITPDGSMLFRVTINS
GGTGGSGGTGGSMVFTLEDFVGDWEQTAAYNLDQVLEQGGVSSLL
QNLAVSVTPIQRIVRSGENALKIDIHVIIPYEGLRPLTEVEMDHY
REPFLNPVDREPLWRFPNELPIAGEPANIVALVEEYMDWLHQSPV
PKLLFWGTPGVLIPPAEAARLAKSLPNCKAVDIGPGLNLLQEDNP
DLIGSEIARWLSTLEISG
HaloTag-178-cpLgBiT+4 67/68-
(E4D, Q42M, M106K, T144D)179
SEQ ID NO: 92
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSA
DQMAQIEEVFKVVYPVDDHHFKVILPYGTLVIDGVTPNKLNYFGR
PYEGIAVFDGKKITVTGTLWNGNKIIDERLIDPDGSMLFRVTINS
GGTGGSGGTGGSMVFTLDDFVGDWEQTAAYNLDQVLEQGGVSSLL
QNLAVSVTPIMRIVRSGENALKIDIHVIIPYEGLRPLTEVEMDHY
REPFLNPVDREPLWRFPNELPIAGEPANIVALVEEYMDWLHQSPV
PKLLFWGTPGVLIPPAEAARLAKSLPNCKAVDIGPGLNLLQEDNP
DLIGSEIARWLSTLEISG
HaloTag-178-(L1-3)cpLgBiT+4 67/68
(E4D, Q42M, M106K, T144D)-179
SEQ ID NO: 93
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
Halo Tag-178-(L1-3; L3-3)cpLgBiT+4 67/68
(E4D, Q42M, M106K, T144D)-179
SEQ ID NO: 94
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLGGSRPLTE
VEMDHYREPFLNPVDREPLWRFPNELPIAGEPANIVALVEEYMDW
LHQSPVPKLLFWGTPGVLIPPAEAARLAKSLPNCKAVDIGPGLNL
LQEDNPDLIGSEIARWLSTLEISG
HaloTag-178-(L1-15)cpLgBiT+4 67/68
(E4D, Q42M, M106K, T144D)-179
SEQ ID NO: 95
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGGSSSGGSADQMAQIEEVFKVVYPVDDHHFKVILPYGTL
VIDGVTPNKLNYFGRPYEGIAVFDGKKITVTGTLWNGNKIIDERL
IDPDGSMLFRVTINSGGTGGSGGTGGSMVFTLDDFVGDWEQTAAY
NLDQVLEQGGVSSLLQNLAVSVTPIMRIVRSGENALKIDIHVIIP
YEGLRPLTEVEMDHYREPFLNPVDREPLWRFPNELPIAGEPANIV
ALVEEYMDWLHQSPVPKLLFWGTPGVLIPPAEAARLAKSLPNCKA
VDIGPGLNLLQEDNPDLIGSEIARWLSTLEISG
HaloTag-178-cpLgBiT+4 49/50
(E4D, Q42M, M106K, T144D)-179
SEQ ID NO: 96
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGE
NALKIDIHVIIPYEGLSADQMAQIEEVFKVVYPVDDHHFKVILPY
GTLVIDGVTPNKLNYFGRPYEGIAVFDGKKITVTGTLWNGNKIID
ERLIDPDGSMLFRVTINSGGTGGSGGTGGSMVFTLDDFVGDWEQT
AAYNLDQVLEQGGVSSLLQNLAVSVTPIMRIVRSRPLTEVEMDHY
REPFLNPVDREPLWRFPNELPIAGEPANIVALVEEYMDWLHQSPV
PKLLFWGTPGVLIPPAEAARLAKSLPNCKAVDIGPGLNLLQEDNP
DLIGSEIARWLSTLEISG
HaloTag-178-(L1-3)cpLgBiT+4 49/50
(E4D, Q42M, M106K, T144D)-179
SEQ ID NO: 97
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGENALKIDIHVIIPYEGLSADQMAQIEEVFKVVYPVDDHHFKVI
LPYGTLVIDGVTPNKLNYFGRPYEGIAVFDGKKITVTGTLWNGNK
IIDERLIDPDGSMLFRVTINSGGTGGSGGTGGSMVFTLDDFVGDW
EQTAAYNLDQVLEQGGVSSLLQNLAVSVTPIMRIVRSRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3; L3-3)cpLgBiT+4 49/50
(E4D, Q42M, M106K, T144D)-179
SEQ ID NO: 98
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGENALKIDIHVIIPYEGLSADQMAQIEEVFKVVYPVDDHHFKVI
LPYGTLVIDGVTPNKLNYFGRPYEGIAVFDGKKITVTGTLWNGNK
IIDERLIDPDGSMLFRVTINSGGTGGSGGTGGSMVFTLDDFVGDW
EQTAAYNLDQVLEQGGVSSLLQNLAVSVTPIMRIVRSGGSRPLTE
VEMDHYREPFLNPVDREPLWRFPNELPIAGEPANIVALVEEYMDW
LHQSPVPKLLFWGTPGVLIPPAEAARLAKSLPNCKAVDIGPGLNL
LQEDNPDLIGSEIARWLSTLEISG
HaloTag-178-(L1-15)cpLgBiT+4 49/50
(E4D, Q42M, M106K, T144D)-179
SEQ ID NO: 99
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGGSSSGGGENALKIDIHVIIPYEGLSADQMAQIEEVFKV
VYPVDDHHFKVILPYGTLVIDGVTPNKLNYFGRPYEGIAVFDGKK
ITVTGTLWNGNKIIDERLIDPDGSMLFRVTINSGGTGGSGGTGGS
MVFTLDDFVGDWEQTAAYNLDQVLEQGGVSSLLQNLAVSVTPIMR
IVRSRPLTEVEMDHYREPFLNPVDREPLWRFPNELPIAGEPANIV
ALVEEYMDWLHQSPVPKLLFWGTPGVLIPPAEAARLAKSLPNCKA
VDIGPGLNLLQEDNPDLIGSEIARWLSTLEISG
HaloTag-194-(L1-3)cpLgBiT+4 67/68
(E4D, Q42M, M106K, T144D)-195
SEQ ID NO: 100
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLGGSSADQMAQIEEVFKVVYPVDDHHFKVILP
YGTLVIDGVTPNKLNYFGRPYEGIAVFDGKKITVTGTLWNGNKII
DERLIDPDGSMLFRVTINSGGTGGSGGTGGSMVFTLDDFVGDWEQ
TAAYNLDQVLEQGGVSSLLQNLAVSVTPIMRIVRSGENALKIDIH
VIIPYEGLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, K123E)-179
SEQ ID NO: 101
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGRPYEGIAVFDGEKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, K123E, R112H)-179
SEQ ID NO: 102
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGHPYEGIAVFDGEKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, K123E, V127T)-179
SEQ ID NO: 103
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGRPYEGIAVFDGEKITTTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, R112H)-179
SEQ ID NO: 104
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGHPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
Halo Tag-178-(L1-3)cpLgBiT+4 67/68
(E4D, Q42M, M106K, T144D, R112H, V127T)-179
SEQ ID NO: 105
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGHPYEGIAVFDGKKITTTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, R112H, V127T, K123E)-
179
SEQ ID NO: 106
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGHPYEGIAVFDGEKITTTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, V127T)-179
SEQ ID NO: 107
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGRPYEGIAVFDGKKITTTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, H86R)-179
SEQ ID NO: 108
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDRHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, H86R, L142R)-179
SEQ ID NO: 109
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDRHFKVILPYGTLVIDGVTPNKLNY
FGRPYEGIAVFDGKKITVTGTLWNGNKIIDERRIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, I73L, L142R)-179
SEQ ID NO: 110
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQLEEVFKVVYPVDDHHFKVILPYGTLVIDGVTPNKLNY
FGRPYEGIAVEDGKKITVTGTLWNGNKIIDERRIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
Halo Tag-178-(L1-3)cpLgBiT+4 67/68
(E4D, Q42M, M106K, T144D, I73L, L142R, H86R)-179
SEQ ID NO: 111
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQLEEVFKVVYPVDDRHFKVILPYGTLVIDGVTPNKLNY
FGRPYEGIAVFDGKKITVTGTLWNGNKIIDERRIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, I73L, H86R)-179
SEQ ID NO: 112
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQLEEVFKVVYPVDDRHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, I73L)-179
SEQ ID NO: 113
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQLEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-3)cpLgBiT+4 67/68
(E4D, Q42M, M106K, T144D, L142R)-179
SEQ ID NO: 114
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGRPYEGIAVFDGKKITVTGTLWNGNKIIDERRIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-GPR)cpLgBiT+4 67/68
(E4D, Q42M, M106K, T144D)-179
SEQ ID NO: 115
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGP
RSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-GRP)cpLgBiT+4 67/68
(E4D, Q42M, M106K, T144D)-179
SEQ ID NO: 116
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGR
PSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-RPG)cpLgBiT+4 67/68
(E4D, Q42M, M106K, T144D)-179
SEQ ID NO: 117
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
GSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-VPR)cpLgBiT+4 67/68
(E4D, Q42M, M106K, T144D)-179
SEQ ID NO: 118
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVVP
RSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-VRP)cpLgBiT+4 67/68
(E4D, Q42M, M106K, T144D)-179
SEQ ID NO: 119
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVVR
PSADQMAQIEEVFKVVYPVDDHHFKVILPYGTLVIDGVTPNKLNY
FGRPYEGIAVFDGKKITVTGTLWNGNKIIDERLIDPDGSMLFRVT
INSGGTGGSGGTGGSMVFTLDDFVGDWEQTAAYNLDQVLEQGGVS
SLLQNLAVSVTPIMRIVRSGENALKIDIHVIIPYEGLRPLTEVEM
DHYREPFLNPVDREPLWRFPNELPIAGEPANIVALVEEYMDWLHQ
SPVPKLLFWGTPGVLIPPAEAARLAKSLPNCKAVDIGPGLNLLQE
DNPDLIGSEIARWLSTLEISG
HaloTag-178-(L1-13*)cpLgBiT+4 67/68
(E4D, Q42M, M106K, T144D)-179-
*Linker EPTTEDLYFQSDN
SEQ ID NO: 120
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVEP
TTEDLYFQSDNSADQMAQIEEVFKVVYPVDDHHFKVILPYGTLVI
DGVTPNKLNYFGRPYEGIAVFDGKKITVTGTLWNGNKIIDERLID
PDGSMLFRVTINSGGTGGSGGTGGSMVFTLDDFVGDWEQTAAYNL
DQVLEQGGVSSLLQNLAVSVTPIMRIVRSGENALKIDIHVIIPYE
GLRPLTEVEMDHYREPFLNPVDREPLWRFPNELPIAGEPANIVAL
VEEYMDWLHQSPVPKLLFWGTPGVLIPPAEAARLAKSLPNCKAVD
IGPGLNLLQEDNPDLIGSEIARWLSTLEISG
Halo Tag-178-cpNLuc67/68-179-NLuc
SEQ ID NO: 121
MAEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSG
DQMGQIEKIFKVVYPVDDHHFKVILHYGTLVIDGVTPNMIDYFGR
PYEGIAVFDGKKITVTGTLWNGNKIIDERLINPDGSLLFRVTING
VTGWRLCERILAGGTGGSGGTGGSMVFTLEDFVGDWRQTAGYNLD
QVLEQGGVSSLFQNLGVSVTPIQRIVLSGENGLKIDIHVIIPYEG
LRPLTEVEMDHYREPFLNPVDREPLWRFPNELPIAGEPANIVALV
EEYMDWLHQSPVPKLLFWGTPGVLIPPAEAARLAKSLPNCKAVDI
GPGLNLLQEDNPDLIGSEIARWLSTLEISGGSGMVFTLEDFVGDW
RQTAGYNLDQVLEQGGVSSLFQNLGVSVTPIQRIVLSGENGLKID
IHVIIPYEGLSGDQMGQIEKIFKVVYPVDDHHFKVILHYGTLVID
GVTPNMIDYFGRPYEGIAVFDGKKITVTGTLWNGNKIIDERLINP
DGSLLFRVTINGVTGWRLCERILAGS
cpHaloTag178/179-cpNLuc67/68
SEQ ID NO: 122
MLTEVEMDHYREPFLNPVDREPLWRFPNELPIAGEPANIVALVEE
YMDWLHQSPVPKLLFWGTPGVLIPPAEAARLAKSLPNCKAVDIGP
GLNLLQEDNPDLIGSEIARWLSTLEISGGSSGGGSSGGEPTTENL
YFQSDNGSSGGGSSGGMAEIGTGFPFDPHYVEVLGERMHYVDVGP
RDGTPVLFLHGNPTSSYVWRNIIPHVAPTHRCIAPDLIGMGKSDK
PDLGYFFDDHVRFMDAFIEALGLEEVVLVIHDWGSALGFHWAKRN
PERVKGIAFMEFIRPIPTWDEWPEFARETFQAFRTTDVGRKLIID
QNVFIEGTLPMGVVRPSGDQMGQIEKIFKVVYPVDDHHFKVILHY
GTLVIDGVTPNMIDYFGRPYEGIAVFDGKKITVTGTLWNGNKIID
ERLINPDGSLLFRVTINGVTGWRLCERILAGGTGGSGGTGGSMVF
TLEDFVGDWRQTAGYNLDQVLEQGGVSSLFQNLGVSVTPIQRIVL
SGENGLKIDIHVIIPYEGL
cpHT178/179-cpNLuc49/50
SEQ ID NO: 123
MLTEVEMDHYREPFLNPVDREPLWRFPNELPIAGEPANIVALVEE
YMDWLHQSPVPKLLFWGTPGVLIPPAEAARLAKSLPNCKAVDIGP
GLNLLQEDNPDLIGSEIARWLSTLEISGGSSGGGSSGGEPTTENL
YFQSDNGSSGGGSSGGMAEIGTGFPFDPHYVEVLGERMHYVDVGP
RDGTPVLFLHGNPTSSYVWRNIIPHVAPTHRCIAPDLIGMGKSDK
PDLGYFFDDHVRFMDAFIEALGLEEVVLVIHDWGSALGFHWAKRN
PERVKGIAFMEFIRPIPTWDEWPEFARETFQAFRTTDVGRKLIID
QNVFIEGTLPMGVVRPGENGLKIDIHVIIPYEGLSGDQMGQIEKI
FKVVYPVDDHHFKVILHYGTLVIDGVTPNMIDYFGRPYEGIAVFD
GKKITVTGTLWNGNKIIDERLINPDGSLLFRVTINGVTGWRLCER
ILAGGTGGSGGTGGSMVFTLEDFVGDWRQTAGYNLDQVLEQGGVS
SLFQNLGVSVTPIQRIVLS
HaloTag-178-cpLgTrip67/68-179
SEQ ID NO: 124
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVSA
DQMAQIEEVFKVVYPVDDHHFKVILPYGTLVIDGVTPNKLNYFGH
PYEGIAVFDGEKITTTGTLWNGNKIIDERLIDPDGGTGGSGGTGS
MVFTLDDFVGDWEQTAAYNLDQVLEQGGVSSLLQNLAVSVTPIMR
IVRSGENALKIDIHVIIPYEGLRPLTEVEMDHYREPFLNPVDREP
LWRFPNELPIAGEPANIVALVEEYMDWLHQSPVPKLLFWGTPGVL
IPPAEAARLAKSLPNCKAVDIGPGLNLLQEDNPDLIGSEIARWLS
TLEISG
HaloTag-178-(L1-3)cpLgTrip67/68-179
SEQ ID NO: 125
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGHPYEGIAVFDGEKITTTGTLWNGNKIIDERLIDPDGGTGGSGG
TGGSMVFTLDDFVGDWEQTAAYNLDQVLEQGGVSSLLQNLAVSVT
PIMRIVRSGENALKIDIHVIIPYEGLRPLTEVEMDHYREPFLNPV
DREPLWRFPNELPIAGEPANIVALVEEYMDWLHQSPVPKLLFWGT
PGVLIPPAEAARLAKSLPNCKAVDIGPGLNLLQEDNPDLIGSEIA
RWLSTLEISG
HaloTag-178-(L1-3; L3-3)cpLgTrip67/68-179
SEQ ID NO: 126
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SSADQMAQIEEVFKVVYPVDDHHFKVILPYGTLVIDGVTPNKLNY
FGHPYEGIAVFDGEKITTTGTLWNGNKIIDERLIDPDGGTGGSGG
TGGSMVFTLDDFVGDWEQTAAYNLDQVLEQGGVSSLLQNLAVSVT
PIMRIVRSGENALKIDIHVIIPYEGLGGSRPLTEVEMDHYREPFL
NPVDREPLWRFPNELPIAGEPANIVALVEEYMDWLHQSPVPKLLF
WGTPGVLIPPAEAARLAKSLPNCKAVDIGPGLNLLQEDNPDLIGS
EIARWLSTLEISG
HaloTag-178-(L1-15)cpLgTrip67/68-179
SEQ ID NO: 127
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGGSSSGGSADQMAQIEEVFKVVYPVDDHHFKVILPYGTL
VIDGVTPNKLNYFGHPYEGIAVEDGEKITTTGTLWNGNKIIDERL
IDPDGGTGGSGGTGGSMVFTLDDFVGDWEQTAAYNLDQVLEQGGV
SSLLQNLAVSVTPIMRIVRSGENALKIDIHVIIPYEGLRPLTEVE
MDHYREPFLNPVDREPLWRFPNELPIAGEPANIVALVEEYMDWLH
QSPVPKLLFWGTPGVLIPPAEAARLAKSLPNCKAVDIGPGLNLLQ
EDNPDLIGSEIARWLSTLEISG
Halo Tag-178-cpLgTrip49/50-179
SEQ ID NO: 128
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVDD
THR.KCPEDRHPCHHPV.RSERRPNGPDRRGV.GGVPCG.SSL.G
DPALWHTGNRRGYAEQAELFRTPV.RHRRVRRREDHYHRDPVERQ
QNYRRAPDRSRRRNRWQRWNREHGLHTRRFRWGLGTDSRLQPGPS
P.TGRCVQFAAESRRVRNSDHEDCPEPLVEEYMDWLHQSPVPKLL
FWGTPGVLIPPAEAARLAKSLPNCKAVDIGPGLNLLQEDNPDLIG
SEIARWLSTLEISG
Halo Tag-178-(L1-3)cpLgTrip49/50-179
SEQ ID NO: 129
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGENALKIDIHVIIPYEGLSADQMAQIEEVFKVVYPVDDHHFKVI
LPYGTLVIDGVTPNKLNYFGHPYEGIAVEDGEKITTTGTLWNGNK
IIDERLIDPDGGTGGSGGTGGSMVFTLDDFVGDWEQTAAYNLDQV
LEQGGVSSLLQNLAVSVTPIMRIVRSRPLTEVEMDHYREPFLNPV
DREPLWRFPNELPIAGEPANIVALVEEYMDWLHQSPVPKLLFWGT
PGVLIPPAEAARLAKSLPNCKAVDIGPGLNLLQEDNPDLIGSEIA
RWLSTLEISG
HaloTag-178-(L1-3; L3-3)cpLgTrip49/50-179
SEQ ID NO: 130
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGENALKIDIHVIIPYEGLSADQMAQIEEVFKVVYPVDDHHFKVI
LPYGTLVIDGVTPNKLNYFGHPYEGIAVFDGEKITTTGTLWNGNK
IIDERLIDPDGGTGGSGGTGGSMVFTLDDFVGDWEQTAAYNLDQV
LEQGGVSSLLQNLAVSVTPIMRIVRSGGSRPLTEVEMDHYREPFL
NPVDREPLWRFPNELPIAGEPANIVALVEEYMDWLHQSPVPKLLF
WGTPGVLIPPAEAARLAKSLPNCKAVDIGPGLNLLQEDNPDLIGS
EIARWLSTLEISG
HaloTag-178-(L1-15)cpLgTrip49/50-179
SEQ ID NO: 131
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVGG
SGGGGSGGSSSGGGENALKIDIHVIIPYEGLSADQMAQIEEVFKV
VYPVDDHHFKVILPYGTLVIDGVTPNKLNYFGHPYEGIAVFDGEK
ITTTGTLWNGNKIIDERLIDPDGGTGGSGGTGGSMVFTLDDFVGD
WEQTAAYNLDQVLEQGGVSSLLQNLAVSVTPIMRIVRSRPLTEVE
MDHYREPFLNPVDREPLWRFPNELPIAGEPANIVALVEEYMDWLH
QSPVPKLLFWGTPGVLIPPAEAARLAKSLPNCKAVDIGPGLNLLQ
EDNPDLIGSEIARWLSTLEISG

1. A composition comprising a polypeptide having at least 70% sequence identity with one of SEQ ID NO: 2-5, wherein each of X₁-X₂₅ is independently selected from any amino acid or absent, wherein at least 5 of X₁-X₂₅ are not absent, wherein the polypeptide has less than 100% sequence identity with SEQ ID NO: 1.

2-4. (canceled)

5. The composition of claim 1, wherein at least 10 of X₁-X₂₅ are not absent.

6. A composition comprising a polypeptide having;
(a) an N-terminal segment comprising at least 70% sequence identity with SEQ ID NO: 6, a C-terminal segment comprising at least 70% sequence identity with SEQ ID NO 10;
(b) an N-terminal segment comprising at least 70% sequence identity with SEQ ID NO: 7, a C-terminal segment comprising at least 70% sequence identity with SEQ ID NO 11;
(c) an N-terminal segment comprising at least 70% sequence identity with SEQ ID NO: 8, a C-terminal segment comprising at least 70% sequence identity with SEQ ID NO 12;
(d) an N-terminal segment comprising at least 70% sequence identity with SEQ ID NO: 9, a C-terminal segment comprising at least 70% sequence identity with SEQ ID NO 13;
(e) an N-terminal segment comprising at least 70% sequence identity with SEQ ID NO: 14, a C-terminal segment comprising at least 70% sequence identity with SEQ ID NO 21;
(f) an N-terminal segment comprising at least 70% sequence identity with SEQ ID NO: 15, a C-terminal segment comprising at least 70% sequence identity with SEQ ID NO 22;
(g) an N-terminal segment comprising at least 70% sequence identity with SEQ ID NO: 16, a C-terminal segment comprising at least 70% sequence identity with SEQ ID NO 23;
(h) an N-terminal segment comprising at least 70% sequence identity with SEQ ID NO: 17, a C-terminal segment comprising at least 70% sequence identity with SEQ ID NO 24;
(i) an N-terminal segment comprising at least 70% sequence identity with SEQ ID NO: 18, a C-terminal segment comprising at least 70% sequence identity with SEQ ID NO 25;
(j) an N-terminal segment comprising at least 70% sequence identity with SEQ ID NO: 19, a C-terminal segment comprising at least 70% sequence identity with SEQ ID NO 26;
(k) an N-terminal segment comprising at least 70% sequence identity with SEQ ID NO: 20, a C-terminal segment comprising at least 70% sequence identity with SEQ ID NO 27;
(l) an N-terminal segment comprising at least 70% sequence identity with SEQ ID NO: 81, a C-terminal segment comprising at least 70% sequence identity with SEQ ID NO 86;
(m) an N-terminal segment comprising at least 70% sequence identity with SEQ ID NO: 82, a C-terminal segment comprising at least 70% sequence identity with SEQ ID NO 87;
(n) an N-terminal segment comprising at least 70% sequence identity with SEQ ID NO: 83, a C-terminal segment comprising at least 70% sequence identity with SEQ ID NO 88;
(o) an N-terminal segment comprising at least 70% sequence identity with SEQ ID NO: 84, a C-terminal segment comprising at least 70% sequence identity with SEQ ID NO 89; or
(p) an N-terminal segment comprising at least 70% sequence identity with SEQ ID NO: 85, a C-terminal segment comprising at least 70% sequence identity with SEQ ID NO 90;
and an internal segment linking the N-terminal and C-terminal segments, wherein the internal segment is greater than 25 amino acids in length.

7-24. (canceled)

25. The composition of claim 6, wherein the internal segment is less than 1000 amino acids in length.

26. The composition of claim 6, wherein the internal segment is a fluorescent or bioluminescent polypeptide capable of emitting energy at a first wavelength.

27. The composition of claim 6, wherein the internal segment is a component of a bioluminescent complex capable of emitting energy at a first wavelength when contacted by one or more complementary components of the bioluminescent complex and a luminophore.

28. The composition of claim 6, wherein the internal segment is binding protein, an enzyme, or an epitope capable of being recognized by a binding protein.

29. The composition of claim 6, wherein the internal segment comprises at least 70% sequence identity with one of SEQ ID NOS: 28-32 or a circularly permuted variant variates thereof.

30. The composition of claim 29, wherein the internal segment comprises one of SEQ ID NOS: 28-32 or a circularly permuted variant thereof.

31-37. (canceled)

38. A method comprising contacting a composition of claim 29 with a luminophore substrate that emits luminescence when contacted by a portion of the polypeptide.

39. The method of claim 38, wherein the luminophore substrate is a coelenterazine substrate or derivative thereof.

40. The method of claim 38, further comprising contacting a composition of claim 37 with a substrate of formula (I):
R-linker-A-X,
wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide.

41. A composition comprising a polypeptide having at least 70% sequence identity with one of SEQ ID NOS: 91-120.

42. A method comprising contacting a composition of claim 41 with peptide comprising at least 70% sequence identity with SEQ ID NO: 30 and luminophore substrate that emits luminescence when contacted by a complex of the peptide and a portion of the polypeptide.

43. (canceled)

44. The method of claim 42, further comprising contacting a composition of claim 41 with a substrate of formula (I):
R-linker-A-X,
wherein R is a solid surface or functional moiety, wherein the linker is a multiatom straight or branched chain including C, N, S, or O, that optionally comprises one or more rings, wherein A-X is a substrate for a dehalogenase, wherein A is (CH₂)₄₋₂₀ and X is a halide.

45-49. (canceled)
